(19) United States
(12) Patent Application Publication
(10) Pub. No.: US 2015/0344407 A1
(43) Pub. Date: Dec. 3, 2015

(54) FENDILINE DERIVATIVES AND METHODS OF USE THEREOF

(71) Applicant: THE BOARD OF REGENTS OF THE UNIVERSITY OF TEXAS SYSTEM, Austin, TX (US)

(72) Inventors: John F. HANCOCK, Houston, TX (US); Dharini VAN DER HOEVEN, Houston, TX (US); George V. HOLLAND, Houston, TX (US); Disha JAIN, Houston, TX (US)

(21) Appl. No.: 14/422,879

(22) PCT Filed: Aug. 21, 2013
(86) PCT No.: PCT/US2013/055999

§ 371 Date: Feb. 20, 2015

Related U.S. Application Data
(60) Provisional application No. 61/691,510, filed on Aug. 21, 2012.

(51) Int. Cl. C07C 211/28; C07C 211/42; C07C 211/31; C07C 215/08; C07C 229/14; C07C 211/32

(52) U.S. Cl. CPC C07C 211/28; C07C 229/14; C07C 211/32; C07C 211/31; C07C 215/08; C07C 211/42

(57) ABSTRACT

Disclosed herein are novel derivatives of fendiline, including compounds of the formula: wherein the variables are defined herein. Also provided are pharmaceutical compositions, kits and articles of manufacture comprising these derivative compounds. Methods and intermediates useful for making the derivatives, and methods of using the derivatives, for example, for the inhibition of K-Ras plasma membrane localization, and compositions thereof, including for the treatment of cancer, are also provided.

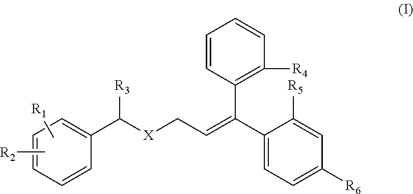

This application claims the benefit of U.S. Provisional Patent Application No. 61/691,510, filed on Aug. 21, 2012, the entirety of which is incorporated herein by reference.

### BACKGROUND OF THE INVENTION

I. Field of the Invention

The present invention relates generally to the fields of biology, chemistry and medicine. In one aspect, it concerns fendiline derivatives for use in the treatment of cancer and other diseases.

II. Description of Related Art

The Ras protein family members belong to a class of proteins called small GTPases. These are involved in transmitting signals within cells (cellular signal transduction). Ras proteins are related in their three dimensional structure and regulate diverse cell behaviors. When Ras is “switched on” by incoming signals, it subsequently switches on other proteins, which ultimately turn on genes involved in cell growth, differentiation and survival. As a result, mutations in ras genes can lead to the production of permanently activated Ras proteins. This can cause inappropriate and overactive signaling inside the cell, even in the absence of incoming signals, which ultimately turn on genes involved in cell growth, differentiation and survival. As a result, mutations in ras genes can lead to the production of permanently activated Ras proteins. Overactive Ras signaling can ultimately lead to cancer. Ras is the most common oncogene in human cancer. Mutations that permanently activate Ras are found in 20-25% of all human tumors and up to 90% in certain types of cancer (e.g., pancreatic cancer). Clinically notable members of the Ras subfamily are H-Ras, N-Ras and K-Ras, mainly for being implicated in many types of cancer. Inappropriate activation of the ras gene has been shown to play a key role in signal transduction, proliferation and malignant transformation.

Localization of K-ras to the plasma membrane is important in order to activate downstream effector pathways (Hancock, 2003; Hancock and Parton, 2005). Fendiline hydrochloride had been identified as specific inhibitor of plasma membrane localization of K-Ras. Fendiline has the following formula:

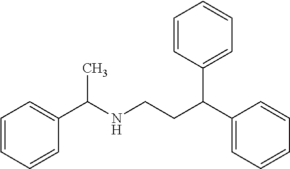 [SMILES: CC(NCCC(c1ccccc1)c1ccccc1)c1ccccc1]

Given these promising properties, that the biological activity profiles of known fendiline derivatives vary, and in view of the wide variety of diseases that may be treated or prevented with compounds that inhibit K-ras localization to the plasma membrane, as well as the degree of unmet medical need represented within this variety of diseases, it is desirable to synthesize new fendiline derivatives with diverse structures that having desirable biological activity profiles for the treatment of one or more indications.

### SUMMARY OF THE INVENTION

In one aspect, the present invention provides novel compounds, including derivatives of fendiline, methods for their manufacture, and methods for their use, including for the treatment and/or prevention of cancer or other diseases.

In one aspect of the invention, compounds of the following formula are provided:

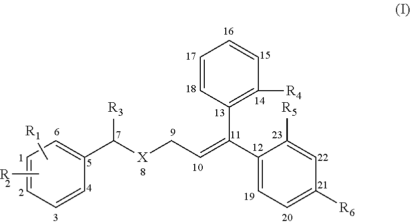

wherein: X is —O— or —NY₁—; wherein Y₁ is hydrogen, alkyl_{(C≦6)}, aralkyl_{(C≦18)}, aralkenyl_{(C≦18)}, or a substituted version of any of these groups; R₁ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; R₂ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, acyl_{(C≦12)}, alkoxy_{(C≦12)}, aryloxy_{(C≦12)}, aralkoxy_{(C≦12)}, heteroaryloxy_{(C≦12)}, acyloxy_{(C≦12)}, alkylamino_{(C<12)}, dialkylamino_{(C<12)}, arylamino_{(C<12)}, aralkylamino_{(C<12)}, heteroarylamino_{(C<12)}, amido_{(C<12)}, or a substituted version of any of these groups; R₁ and R₃, taken together, are alkanediyl_{(C≦6)} or substituted alkanediyl_{(C≦6)} between carbon atoms 6 and 7; R₄ and R₅ are each hydrogen; or R₄ and R₅, taken together, are a covalent single bond, —O—, —S—, alkanediyl_{(C≦12)}, or alkenediyl_{(C≦12)} between carbon atoms 14 and 23; and R₆ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; with the proviso that if R₁ and R₂ are both hydrogen, then R₃ is substituted alkyl_{(C≦6)}; or a pharmaceutically acceptable salt or tautomer thereof.

In some embodiments, the compound is further defined by the formula:

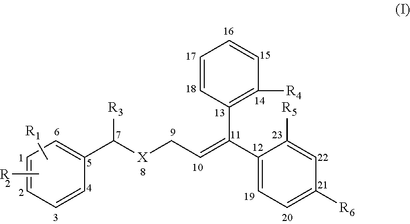

wherein: X is —O— or —NH—; R₁ is hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; R₂ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, acyl_{(C≦12)}, alkoxy_{(C≦12)}, aryloxy_{(C≦12)}, aralkoxy_{(C≦12)}, heteroaryloxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, amido_{(C≦12)}, or a substituted version of any of these groups; R₄ and R₅ are each hydrogen or are taken together and are a covalent single bond, —O—, —S—, alkanediyl_{(C≦12)}, or alkenediyl_{(C≦12)}; and R₆ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; or a pharmaceutically acceptable salt or tautomer thereof.

In some embodiments, X is —NY₁—. In some embodiments, Y₁ is aralkenyl_{(C≦18)}. In some embodiments, Y₁ is

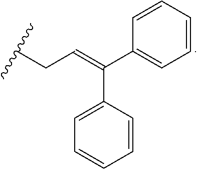 [SMILES: *CC=C(c1ccccc1)c1ccccc1]

In other embodiments, Y₁ is hydrogen. In some embodiments, X is —NH—. In some embodiments, R₁ is amino, halo, or nitro. In some embodiments, R₁ is fluoro, bromo, or chloro. In other embodiments, R₁ is alkoxy_{(C≦6)}. In some embodiment, R₁ is methoxy. In other embodiments, R₁ is alkyl_{(C≦6}). In other embodiments, R₁ is methyl. In other embodiments, R₁ is hydrogen. In other embodiments, Rᵢ and R₃ are taken together and are alkanediyl_{(C≦6)}. In some embodiments, R₂ is hydrogen. In some embodiments, R₃ is alkyl_{(C≦12)} or substituted alkyl_{(C≦12)}. In some embodiments, R₃ is alkyl_{(C≦12)}. In some embodiments, R₃ is methyl. In other embodiments, R₃ is substituted alkyl_{(C≦12)}. In some embodiments, R₃ is —CH₂OH. In some embodiments, R₄ and R₅ are each hydrogen. In other embodiments, R₄ and R₅ are taken together and are a covalent single bond. In other embodiments, R₄ and R₅ are taken together and are —O— or —S—. In other embodiments, R₄ and R₅ are taken together and are alkanediyl_{(C≦12)} or alkenediyl_{(C≦12)}. In some embodiments, R₆ is hydrogen.

In some embodiments, the compounds are further defined as:

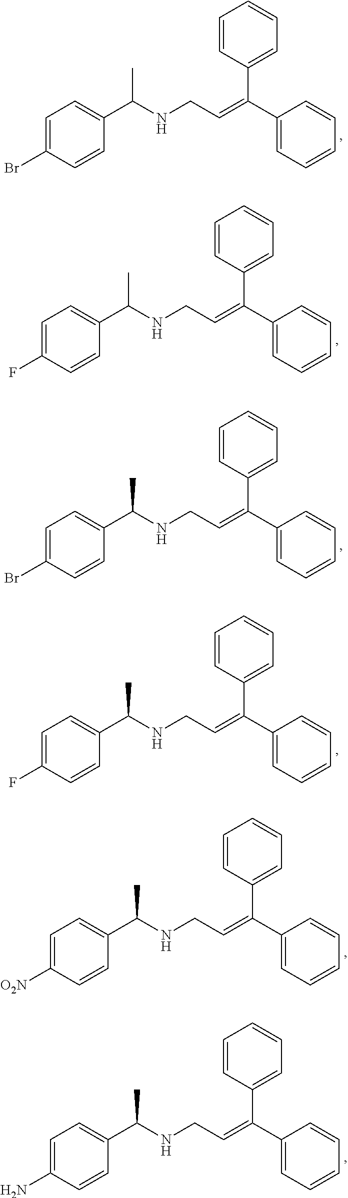 [SMILES: CC(NCC=C(c1ccccc1)c1ccccc1)c1ccc(Br)cc1 ; CC(NCC=C(c1ccccc1)c1ccccc1)c1ccc(F)cc1 ; C[C@@H](NCC=C(c1ccccc1)c1ccccc1)c1ccc(Br)cc1 ; C[C@@H](NCC=C(c1ccccc1)c1ccccc1)c1ccc(F)cc1 ; C[C@@H](NCC=C(c1ccccc1)c1ccccc1)c1ccc([N+](=O)[O-])cc1 ; C[C@@H](NCC=C(c1ccccc1)c1ccccc1)c1ccc(N)cc1]

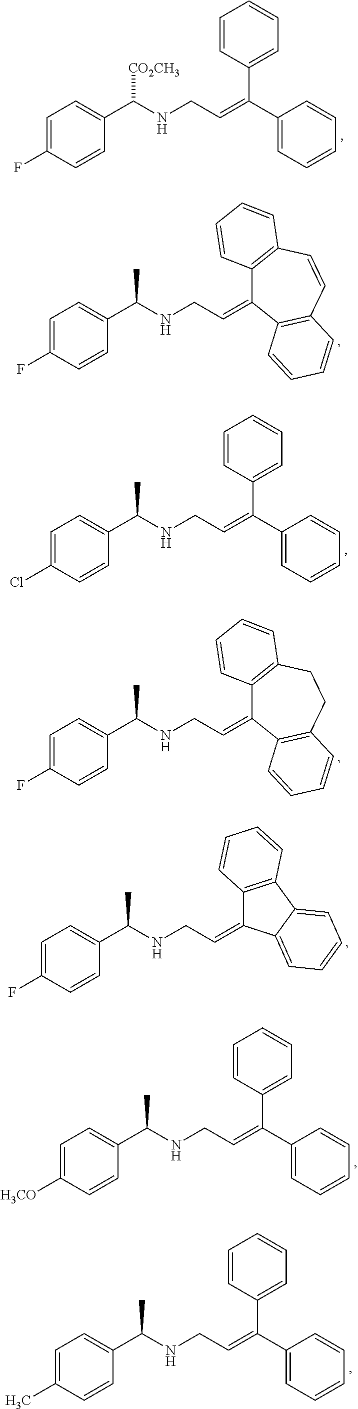

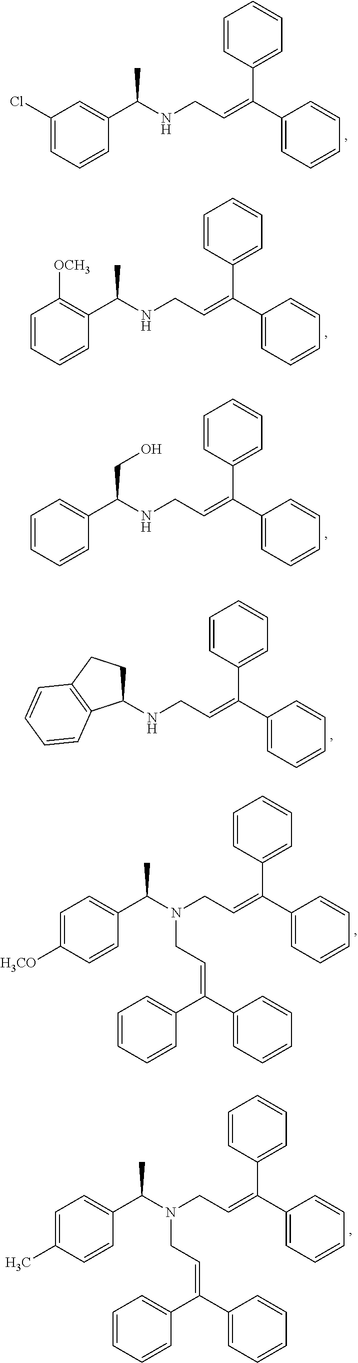

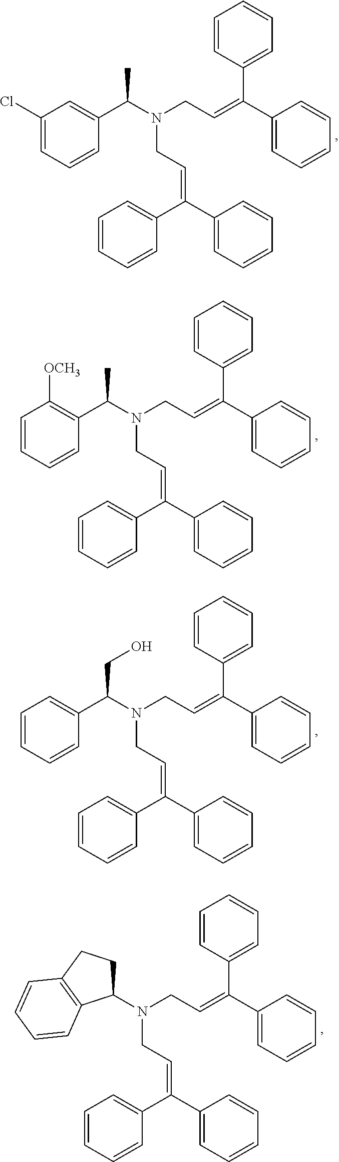

or a pharmaceutically acceptable salt or tautomer thereof.

In other embodiments, the compound is further defined as:

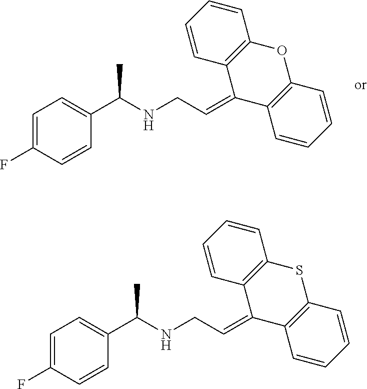 [SMILES: C[C@@H](NCC=C1c2ccccc2Oc2ccccc21)c1ccc(F)cc1 ; C[C@@H](NCC=C1c2ccccc2Sc2ccccc21)c1ccc(F)cc1]

or a pharmaceutically acceptable salt or tautomer thereof.

In another aspect of the invention, there are provided compounds of the formula:

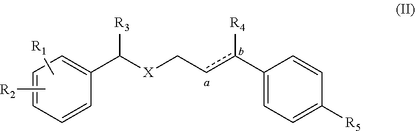

wherein: X is —O— or —NH—; R₁ and R₂ are each independently hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C<6)}, amido_{(C<6)}, or a substituted version of any of these groups; and R₃ is acyl_{(C≦12)}, substituted acyl_{(C≦12)}, heteroaryl_{(C≦12)}, substituted heteroaryl_{(C≦12)}; R₄ is hydrogen or aryl_{(C≦12)}; and R₅ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; or pharmaceutically acceptable salts or tautomers thereof.

In some embodiments, the bond between the atoms labeled a and b is a double bond. In some embodiments, X is —NH—. In some embodiments, R₁ is hydrogen. In other embodiments, R₁ is halo, for example, fluoro. In some embodiments, R₂ is hydrogen. In some embodiments, R₃ is substituted acyl_{(C≦12)}, for example, methoxycarbonyl. In some embodiments, R₃ is heteroaryl_{(C≦12)}, for example:

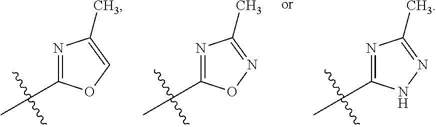

In some embodiments, R₄ is aryl_{(C≦12)}, for example, phenyl. In some embodiments, R₄ is hydrogen. In some embodiments, R₅ is hydrogen.

In some embodiments, the invention provides compounds of the following formulas:

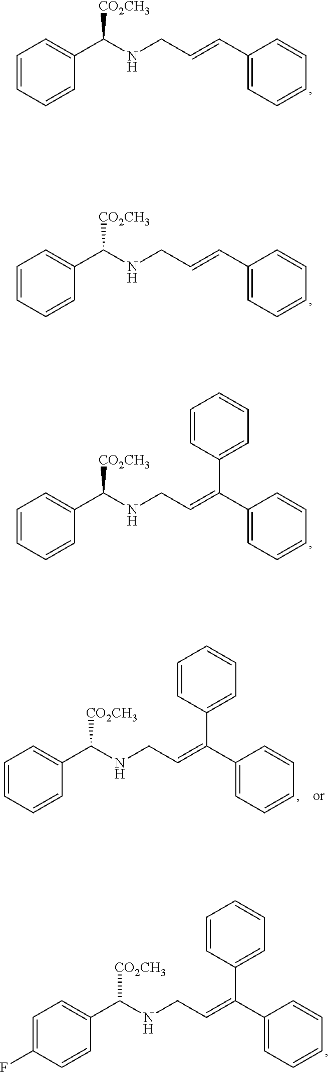 [SMILES: COC(=O)[C@@H](NC/C=C/c1ccccc1)c1ccccc1 ; COC(=O)[C@H](NC/C=C/c1ccccc1)c1ccccc1 ; COC(=O)[C@@H](NCC=C(c1ccccc1)c1ccccc1)c1ccccc1 ; COC(=O)[C@H](NCC=C(c1ccccc1)c1ccccc1)c1ccccc1 ; COC(=O)[C@H](NCC=C(c1ccccc1)c1ccccc1)c1ccc(F)cc1]

or pharmaceutically acceptable salts or tautomers of any of these.

In some embodiments, the invention provides compounds of the following formulas:

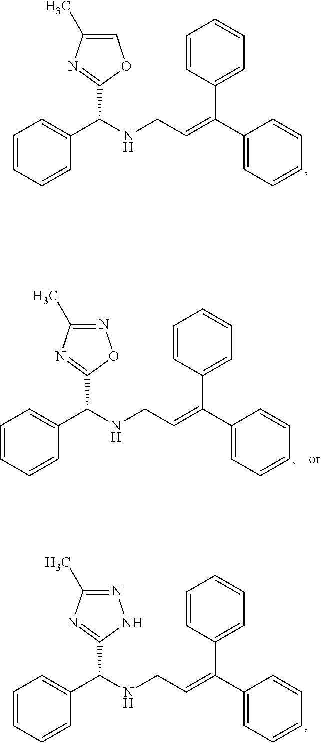 [SMILES: Cc1coc([C@H](NCC=C(c2ccccc2)c2ccccc2)c2ccccc2)n1 ; Cc1noc([C@H](NCC=C(c2ccccc2)c2ccccc2)c2ccccc2)n1 ; Cc1n[nH]c([C@H](NCC=C(c2ccccc2)c2ccccc2)c2ccccc2)n1]

or pharmaceutically acceptable salts or tautomers of any of these.

In another aspect of the invention, there are provided compounds of the formula:

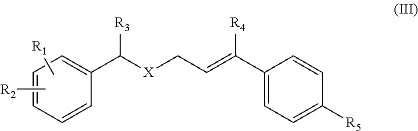

wherein: X is —O— or —NH—; R₁ and R₂ are each independently hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; and R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, acyl_{(C≦12)}, alkoxy_{(C≦12)}, aryloxy_{(C≦12)}, aralkoxy_{(C≦12)}, heteroaryloxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C<12)}, amido_{(C<12)}, or a substituted version of any of these groups; R₄ is hydrogen or aryl_{(C≦12)}; and R₅ is hydroxy, amino, halo, nitro or cyano; or alkyl_{(C≦6)}, aryl_{(C≦12)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; or pharmaceutically acceptable salts or tautomers thereof.

In some embodiments, X is —NH—. In some embodiments, R₁ is hydrogen. In some embodiments, R₂ is hydrogen. In some embodiments, R₃ is alkyl_{(C≦12)}, for example, methyl. In some embodiments, R₄ is aryl_{(C≦12)}, for example, phenyl. In some embodiments, R₄ is hydrogen. In some embodiments, R₅ is amino or nitro. In some embodiments, R₅ is aryl_{(C≦12)}, for example, phenyl.

In some embodiments, the invention provides compounds of the following formulas:

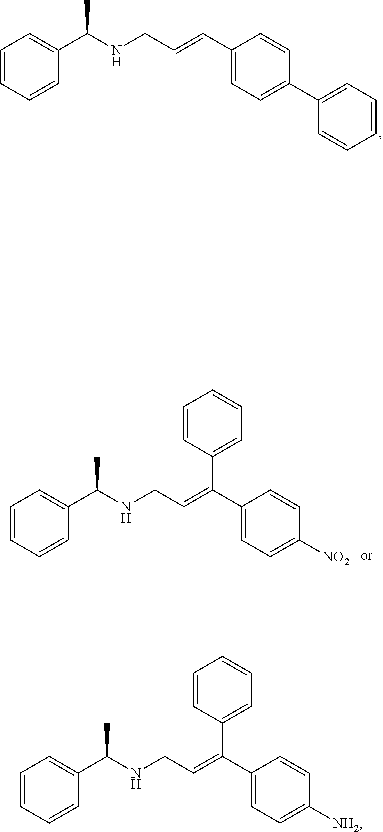 [SMILES: C[C@@H](NC/C=C/c1ccc(-c2ccccc2)cc1)c1ccccc1 ; C[C@@H](NC/C=C(\c1ccccc1)c1ccc([N+](=O)[O-])cc1)c1ccccc1 ; "or" ; C[C@@H](NC/C=C(\c1ccccc1)c1ccc(N)cc1)c1ccccc1]

or pharmaceutically acceptable salts or tautomers of any of these.

In another aspect of the invention, there are provided compounds of the formula:

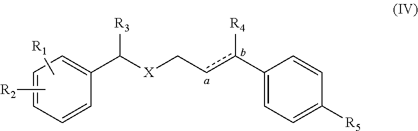

wherein: X is —O—; R₁ and R₂ are each independently hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; and R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, acyl_{(C≦12)}, alkoxy_{(C≦12)}, aryloxy_{(C≦12)}, aralkoxy_{(C≦12)}, heteroaryloxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, amido_{(C≦12)}, or a substituted version of any of these groups; R₄ is hydrogen or aryl_{(C≦12)}; and R₅ is hydrogen, hydroxy, amino, halo, nitro or cyano; or alkyl_{(C<6)}, aryl_{(C<12)}, acyl_{(C<6)}, alkoxy_{(C<6)}, acyloxy_{(C≦6)}, alkylamino_{(C≦6)}, dialkylamino_{(C≦6)}, amido_{(C≦6)}, or a substituted version of any of these groups; or pharmaceutically acceptable salts or tautomers thereof.

In some embodiments, the bond between the atoms labeled a and b is a single bond. In other embodiments, the bond between the atoms labeled a and b is a double bond. In some embodiments, R₁ is hydrogen. In some embodiments, R₂ is hydrogen. In some embodiments, R₃ is alkyl_{(C≦12)}, for example, methyl. In some embodiments, R₄ is aryl_{(C≦12)}, for example, phenyl. In some embodiments, R₄ is hydrogen. In some embodiments, R₅ is hydrogen.

In some embodiments, the invention provides compounds of the following formulas:

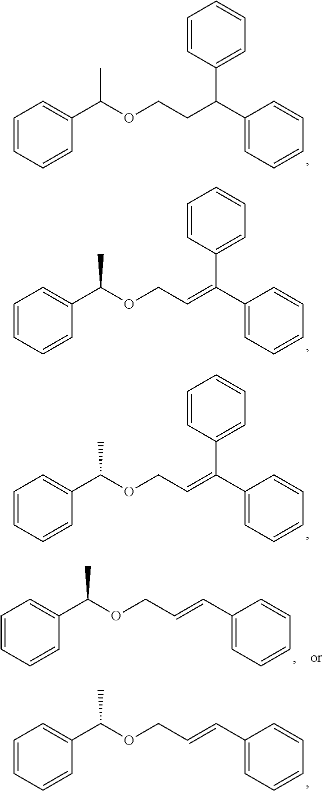

or pharmaceutically acceptable salts or tautomers of any of these.

In another aspect of the invention, there are provided compounds of the formula:

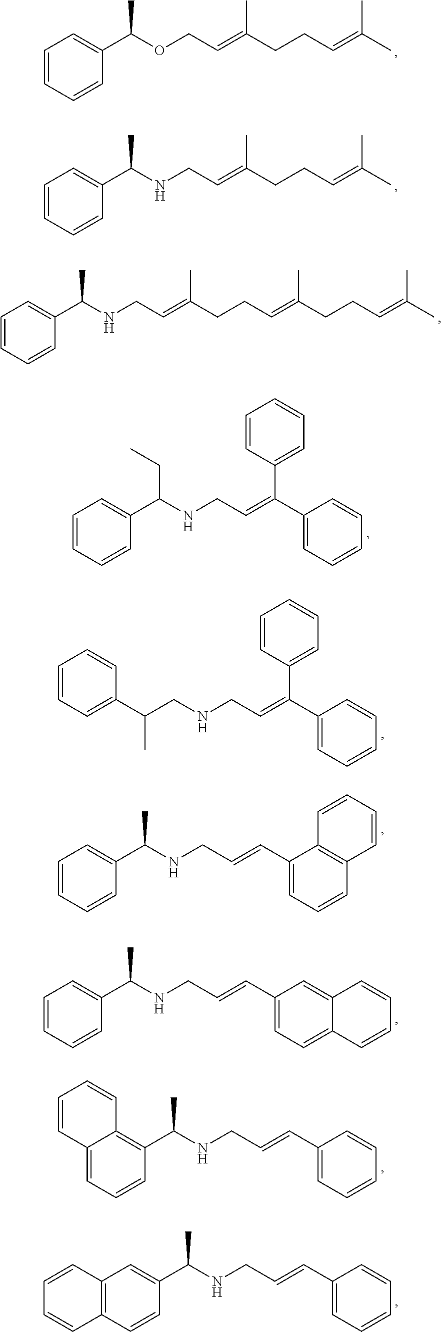

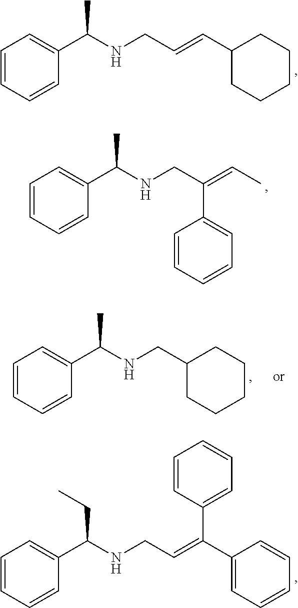

or pharmaceutically acceptable salts or tautomers of any of these.

In another aspect of the invention, there are provided a compound of the formula:

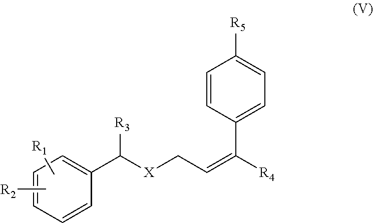

wherein: X is —O— or —NH—; R₁ and R₂ are each independently hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; and R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, acyl_{(C≦12)}, alkoxy_{(C≦12)}, aryloxy_{(C≦12)}, aralkoxy_{(C≦12)}, heteroaryloxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, amido_{(C≦12)}, or a substituted version of any of these groups; R₄ is hydrogen or aryl_{(C≦12)}; and R₅ is hydroxy, amino, halo, nitro or cyano; or alkyl_{(C≦6)}, aryl_{(C≦12)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; or a pharmaceutically acceptable salt or tautomer thereof. In some embodiments, X is —NH—. In some embodiments, R₁ is hydrogen. In some embodiments, R₂ is hydrogen. In some embodiments, R₃ is alkyl_{(C≦12)}, for example, methyl. In some embodiments, R₄ is aryl_{(C≦12)}, for example, phenyl. In other embodiments, R₄ is hydrogen. In some embodiments, R₅ is amino In other embodiments, R₅ is nitro.

In some embodiments, the compound is further defined as:

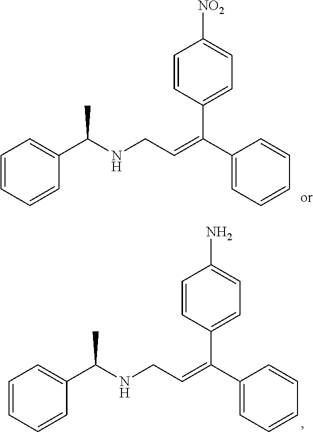 [SMILES: C[C@@H](NC/C=C(/c1ccccc1)c1ccc([N+](=O)[O-])cc1)c1ccccc1 ; C[C@@H](NC/C=C(/c1ccccc1)c1ccc(N)cc1)c1ccccc1]

or a pharmaceutically acceptable salt thereof.

In another aspect of the invention, there are provided compounds of the formula:

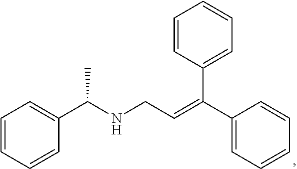 [SMILES: C[C@H](NCC=C(c1ccccc1)c1ccccc1)c1ccccc1]

substantially free from optical isomers thereof or a pharmaceutically acceptable salt thereof.

In some embodiments, the compound is in the form of a pharmaceutically acceptable salt. In some embodiments, the compound is in the form of a hydrochloride salt. In other embodiments, the compound is not a salt.

In another aspect there are provided pharmaceutical compositions comprising a compound disclosed herein and a pharmaceutically acceptable carrier. In some embodiments, the composition is formulated for oral administration. In some embodiments, the compositions further comprise one or more pharmaceutically acceptable excipients. In some embodiments, the composition is formulated for controlled release.

In another aspect there are provided methods of treating a hyperproliferative disorder in a patient, the method comprising administering to a patient in need thereof an effective amount of a compound disclosed herein. In some embodiments, the hyperproliferative disorder is cancer. In some embodiments, the cancer is lung cancer, brain cancer, head & neck cancer, breast cancer, skin cancer, liver cancer, pancreatic cancer, prostate cancer, stomach cancer, colon cancer, rectal cancer, uterine cancer, cervical cancer, ovarian cancer, testicular cancer, skin cancer, oral cancer or esophageal cancer. In some embodiments, the hyperproliferative disorder is leukemia, lymphoma or myeloma. In some embodiments, the hyperproliferative disorder is acute myeloid leukemia, chronic myelogenous leukemia or multiple myeloma. In other embodiments, the cancer is colorectal cancer, pancreatic cancer, uterine cancer, or lung cancer. In other embodiments, the cancer is pancreatic cancer. In other embodiments, the cancer is uterine cancer. In some embodiments, the patient is human.

In yet another aspect, there are provided a method of treating a disease associated with an overactive Ras signaling pathway, wherein the method comprises administering to a patient in need thereof a therapeutically effective amount of the compound of the present disclosure. In some embodiments, the disease is caused by a mutation in the ras gene. In other embodiments, the disease is caused by a Ras protein which is permanently activated. In some embodiments, the Ras protein is H-Ras, N-Ras, or K-Ras. In some embodiments, the Ras protein is K-Ras. In some embodiments, the Ras protein causes changes to cell signal transduction, proliferation, or malignant transformation. In some embodiments, the compound prevents the localization of a Ras protein into the plasma membrane. In some embodiments, the compound prevents the localization of K-Ras into the plasma membrane.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed.

FIGS. 1 & 2—Representative images showing degree of K-ras mislocalization with compound treatment (images corresponds with column 3 of Table 2). MDCK cells stably expressing GFP-tagged K-rasG12V were treated with vehicle or compound (4 μM) for 48 hours and fixed with 4% paraformaldehyde. The coverslips were mounted in Mowiol and imaged by confocal microscopy (Nikon A1) using a 60× objective. The number above each image corresponds the fendiline derivative code. See Tables 1 and 2.

FIG. 3A &B-FIG. 3A Representative images taken in GFP and mCherry channels of the confocal microscope and the corresponding overlay of the two images showing degree of K-Ras mislocalization when treated with increasing concentrations of compound 72 and (FIG. 3B) the corresponding concentration-response (Mander's coefficient) curve. Compound 72 concentration-dependently mislocalizes GFP-K-RasG12V.

FIG. 4A-4E—Concentration-response curves of 42, 68, 70, 72 and 73 on proliferation of five endometrial cancer cell lines. KLE and ESS-1 express wild type K-Ras and Hec-1A, Hec-1B and Hec50 express oncogenic mutant K-Ras. Fendiline derivatives more potently inhibit the proliferation of oncogenic K-Ras-transformed endometrial cancer cell lines. EC₅₀ values for their inhibitory effect are tabulated. ND means that the EC₅₀ value could not be determined within the dose range tested. See also Table 4.

FIG. 5A-5E—Concentration-response curves of 42, 68, 70, 72 and 73 on proliferation of five pancreatic cancer cell lines. BxPC-3 expresses wild type K-Ras, whereas the others express oncogenic mutant K-Ras. Fendiline derivatives more potently inhibit the proliferation of oncogenic K-Ras-transformed pancreatic cancer cell lines. EC₅₀ values for their inhibitory effect are tabulated. ND means that the EC₅₀ value could not be determined within the dose range tested. See also Table 5.

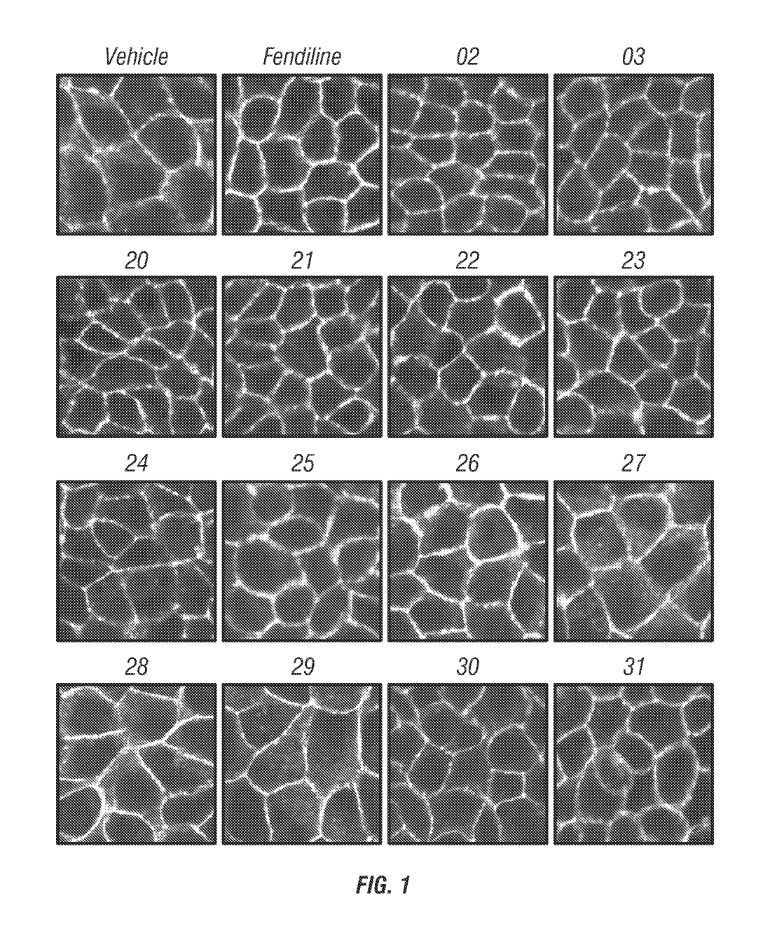

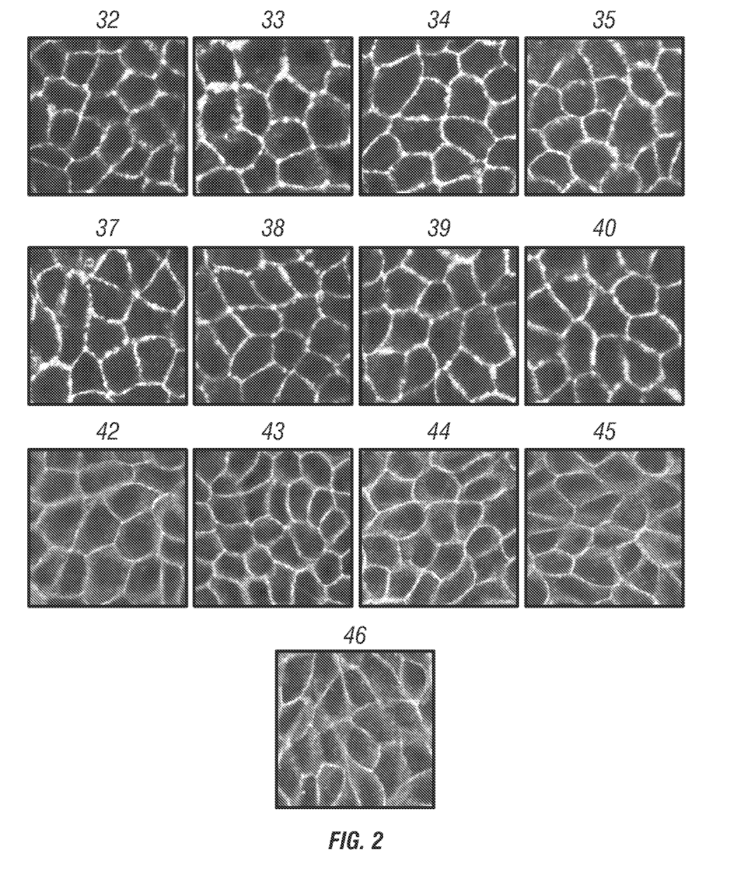

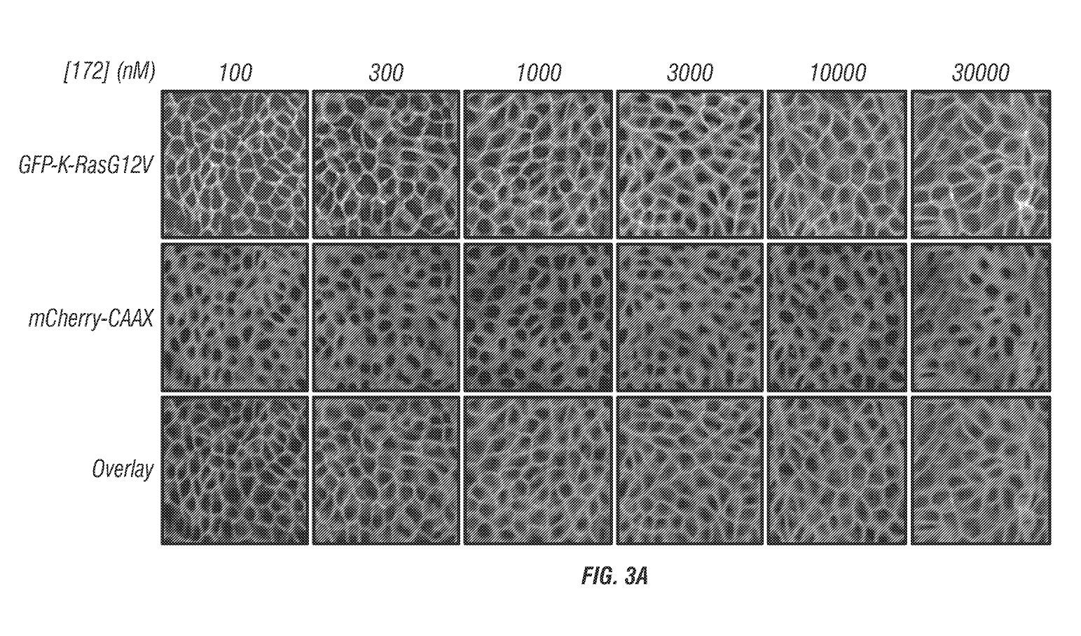

### DETAILED DESCRIPTION OF THE INVENTION

In one aspect, there are disclosed herein fendiline derivatives, methods for their manufacture, and methods for their use, including for the treatment and/or prevention of cancer or other diseases.

### I. DEFINITIONS

When used in the context of a chemical group, “hydrogen” means —H; “hydroxy” means —OH; “oxo” means ═O; “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, for example, the structure

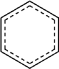

includes the structures

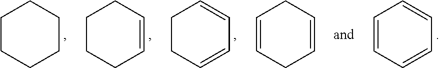

As will be understood by a person of skill in the art, no one such ring atom forms part of more than one double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation (e.g., either R or S) or the geometry is undefined (e.g., either E or Z).

Any undefined valency on an atom of a structure shown in this application implicitly represents a hydrogen atom bonded to the atom. When a group “R” is depicted as a “floating group” on a ring system, for example, in the formula:

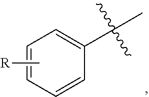

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

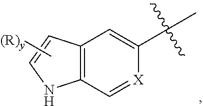

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “saturated” as used herein means the compound or group so modified has no carbon-carbon double and no carbon-carbon triple bonds, except as noted below. The term does not preclude carbon-heteroatom multiple bonds, for example a carbon oxygen double bond or a carbon nitrogen double bond. Moreover, it does not preclude a carbon-carbon double bond that may occur as part of keto-enol tautomerism or imine/enamine tautomerism.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl). When the term “aliphatic” is used without the “substituted” modifier only carbon and hydrogen atoms are present. When the term is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

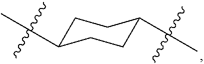 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which one or more hydrogen atoms has been substituted with a halo group and no other atoms aside from carbon, hydrogen and halogen are present. The group, —CH₂Cl is a non-limiting examples of a haloalkyl. An “alkane” refers to the compound H—R, wherein R is alkyl. The term “fluoroalkyl” is a subset of substituted alkyl, in which one or more hydrogen has been substituted with a fluoro group and no other atoms aside from carbon, hydrogen and fluorine are present. The groups, —CHF, —CF₃, and —CH₂CF₃ are non-limiting examples of fluoroalkyl groups. An “alkane” refers to the compound H—R, wherein R is alkyl.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

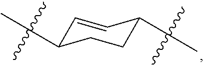 [SMILES: *C1C=CC(*)CC1]

are non-limiting examples of alkenediyl groups. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups, —CH═CHF, —CH═CHCl and —CH═CHBr, are non-limiting examples of substituted alkenyl groups. An “alkene” refers to the compound H—R, wherein R is alkenyl.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. When alkynyl is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. An “alkyne” refers to the compound H—R, wherein R is alkynyl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl group (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and the monovalent group derived from biphenyl. The term “arenediyl” when used without the “substituted” modifier refers to a divalent aromatic group, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of arenediyl groups include:

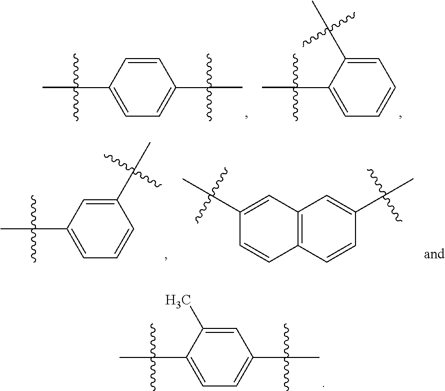

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. An “arene” refers to the compound H—R, wherein R is aryl.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term is used with the “substituted” modifier one or more hydrogen atom from the alkanediyl and/or the aryl has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “aralkenyl” when used without the “substituted” modifier refers to the monovalent group -alkenediyl-aryl, in which the terms alkenediyl and aryl are each used in a manner consistent with the definitions provided above. Non-limiting examples of aralkenyls are: 2-phenylethenyl and 3,3-diphenyl-prop-2-enyl. When the term is used with the “substituted” modifier one or more hydrogen atom from the alkanediyl and/or the aryl has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. Non-limiting examples of substituted aralkenyls are: (3-nitrophenyl)-ethenyl, and 4-cyano-4-phenyl-but-1-enyl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroarenediyl groups include:

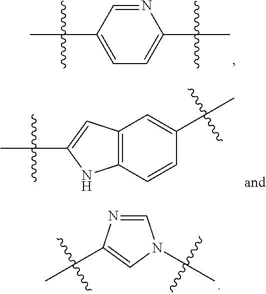

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. As used herein, the term does not preclude the presence of one or more alkyl groups (carbon number limitation permitting) attached to the ring or ring system. If more than one ring is present, the rings may be fused or unfused. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, and pyranyl. When the term “heterocycloalkyl” used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. When either of these terms are used with the “substituted” modifier one or more hydrogen atom (including the hydrogen atom directly attached the carbonyl or thiocarbonyl group) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), —OCH(CH₂)₂, —O-cyclopentyl, and —O-cyclohexyl. The terms “alkenyloxy”, “alkynyloxy”, “aryloxy”, “aralkoxy”, “heteroaryloxy”, and “acyloxy”, when used without the “substituted” modifier, refers to groups, defined as —OR, in which R is alkenyl, alkynyl, aryl, aralkyl, heteroaryl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The term “alcohol” corresponds to an alkane, as defined above, wherein at least one of the hydrogen atoms has been replaced with a hydroxy group.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, and “alkylsulfonylamino” when used without the “substituted” modifier, refers to groups, defined as —NHR, in which R is alkoxy, alkenyl, alkynyl, aryl, aralkyl, heteroaryl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≦15%, more preferably ≦10%, even more preferably ≦5%, or most preferably ≦1% of another stereoisomer(s).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

Other abbreviations used herein are as follows:

¹H NMR Proton Nuclear Magnetic Resonance

CDCl₃ Deuterochloroform

DIBAL-H Di-isobutyl Aluminum Hydride

DCE 1,2-Dichloroethane

DCM Dichloromethane

DMF Dimethylformamide

Et₂O Diethyl ether

ESI(MS) Electrospray Ionization Mass Spectrometry

EtOAc Ethyl acetate

EtOH Ethyl alcohol

g Gram

LAH Lithium aluminum hydride

M Molar

MeOH Methanol

MHz Mega Hertz

μg Microgram

μM Micromolar

mg Milligram

mL Milliliter

mM Millimolar

mmol Millimole

MsCl Methane sulfonyl chloride

N Normal

NaBH(OAc)₃ Sodium Triacetoxyborohydride

ppt Precipitate

RB Round bottom

TEA Triethyl amine

Rt or RT Room temperature

THF Tetrahydrofuran

TLC Thin Layer Chromatography

TsCl p-Toluene sulfonyl chloride

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. FENDILINE DERIVATIVES

The compounds provided by the present disclosure are summarized above in the summary of the invention section and in the claims below. They may be made using the methods provided in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

A non-limiting list of examples of fendiline derivatives provided by the present invention include:

TABLE 1
Fendiline Derivatives

| Formula | Compound Details |
|---|---|
| 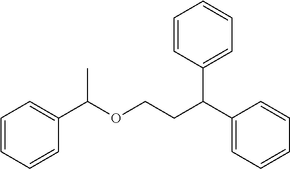 [SMILES: CC(OCCC(c1ccccc1)c1ccccc1)c1ccccc1] | Compound 13 Chemical Name: 3-(1- phenylethoxy)propane- 1,1-diyl)-dibenzene Formula: C₂₃H₂₄O Molecular Weight: 316.44 |
| 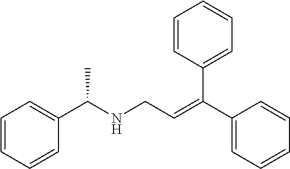 [SMILES: C[C@H](NCC=C(c1ccccc1)c1ccccc1)c1ccccc1] | Compound 17 Chemical Name: (S)-3,3- diphenyl-N-(1- phenylethyl)prop-2-en-1- amine Formula: C₂₃H₂₃N Molecular Weight: 313.44 |
| 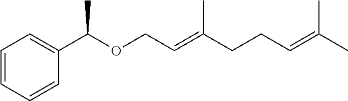 [SMILES: CC(C)=CCC/C(C)=C/CO[C@H](C)c1ccccc1] | Compound 20 Chemical Name: (R,E)-(1- ((3,7-dimethylocta-2,6-dien- 1-yl)oxy)ethyl)benzene Formula: C₁₈H₂₆O Molecular Weight: 258.40 |
| 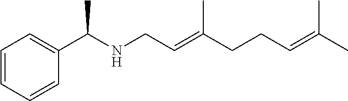 [SMILES: CC(C)=CCC/C(C)=C/CN[C@H](C)c1ccccc1] | Compound 21 Chemical Name: (R,E)-3,7- dimethyl-N-(1- phenylethyl)octa-2,6-dien- 1-amine Formula: C₁₈H₂₇N Molecular Weight: 257.41 |
| 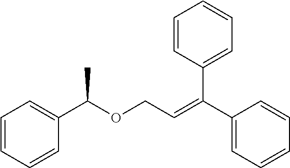 [SMILES: C[C@@H](OCC=C(c1ccccc1)c1ccccc1)c1ccccc1] | Compound 22 Chemical Name: (R)-(3-(1- phenylethoxy)prop-1-ene- 1,1-(diyl)dibenzene Formula: C₂₃H₂₂O Molecular Weight: 314.42 |
| 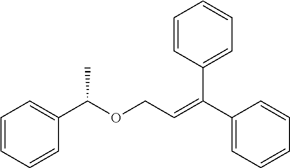 [SMILES: C[C@H](OCC=C(c1ccccc1)c1ccccc1)c1ccccc1] | Compound 23 Chemical Name: (S)-(3-(1- phenylethoxy)prop-1-ene- 1,1-diyl)dibenzene Formula: C₂₃H₂₂O Molecular Weight: 314.42 |
| 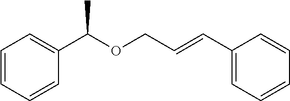 [SMILES: C[C@@H](OC/C=C/c1ccccc1)c1ccccc1] | Compound 24 Chemical Name: (R,E)-(1- (cinnamyloxy)ethyl)benzene Formula: C₁₇H₁₈O Molecular Weight: 238.32 |
| 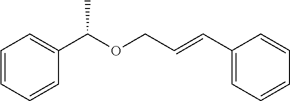 [SMILES: C[C@H](OC/C=C/c1ccccc1)c1ccccc1] | Compound 25 Chemical Name: (S,E)-(1- (cinnamyloxy)ethyl)benzene Formula: C₁₇H₁₈O Molecular Weight: 238.32 |
| 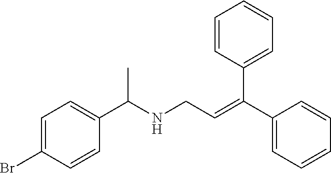 [SMILES: CC(NCC=C(c1ccccc1)c1ccccc1)c1ccc(Br)cc1] | Compound 26 Chemical Name: N-(1-(4- bromophenyl)ethyl)-3,3- diphenylprop-2-en-1-amine Formula: C₂₃H₂₂BrN Molecular Weight: 392.33 |
| 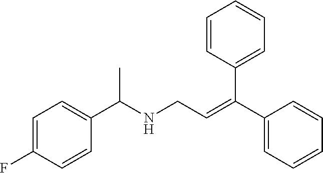 [SMILES: CC(NCC=C(c1ccccc1)c1ccccc1)c1ccc(F)cc1] | Compound 27 Chemical Name: N-(1-(4- fluorophenyl)ethyl)-3,3- diphenylprop-2-en-1-amine Formula: C₂₃H₂₂FN Molecular Weight: 331.43 |
| 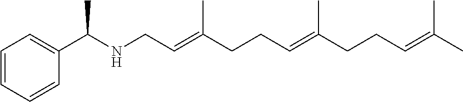 [SMILES: CC(C)=CCC/C(C)=C/CC/C(C)=C/CN[C@H](C)c1ccccc1] | Compound 28 Chemical Name: (2E,6E)- 3,7,11-trimethyl-N-((R)-1- phenylethyl)dodeca- 2,6,10-trien-1-amine Formula: C₂₃H₃₅N Molecular Weight: 325.53 |
| 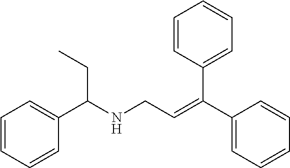 [SMILES: CCC(NCC=C(c1ccccc1)c1ccccc1)c1ccccc1] | Compound 29 Chemical Name: 3,3- diphenyl-N-(1- phenylpropyl)prop-2-en- 1-amine Formula: C₂₄H₂₅N Molecular Weight: 327.46 |
| 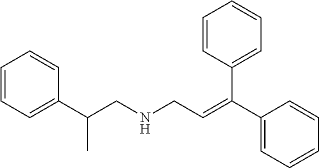 [SMILES: CC(CNCC=C(c1ccccc1)c1ccccc1)c1ccccc1] | Compound 30 Chemical Name: 3,3- diphenyl-N-(2- phenylpropyl)prop-2-en- 1-amine Formula: C₂₄H₂₅N Molecular Weight: 327.46 |
| 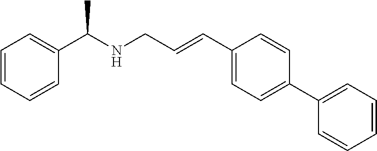 [SMILES: C[C@@H](NC/C=C/c1ccc(-c2ccccc2)cc1)c1ccccc1] | Compound 31 Chemical Name: (R,E)-3- ([1,1′-biphenyl]-4-yl)-N- (1-phenylethyl)prop-2-en- 1-amine Formula: C₂₃H₂₃NCl Molecular Weight: 313.44 |
| 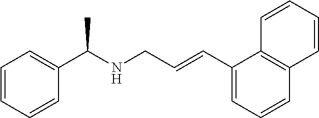 [SMILES: C[C@@H](NC/C=C/c1cccc2ccccc12)c1ccccc1] | Compound 32 Chemical Name: (R,E)-3- (naphthalen-1-yl)-N-(1- phenylethyl)prop-2-en- 1-amine Formula: C₂₁H₂₁N Molecular Weight: 287.40 |
| 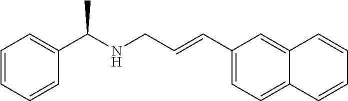 [SMILES: C[C@@H](NC/C=C/c1ccc2ccccc2c1)c1ccccc1] | Compound 33 Chemical Name: (R,E)-3- (naphthalen-2-yl)-N-(1- phenylethyl)prop-2-en- 1-amine Formula: C₂₁H₂₁N Molecular Weight: 287.40 |
| 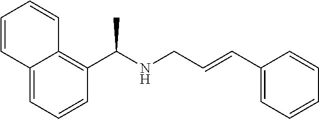 [SMILES: C[C@@H](NC/C=C/c1ccccc1)c1cccc2ccccc12] | Compound 34 Chemical Name: (R,E)-N- (1-(naphthalen-1-yl)ethyl)- 3-phenylprop-2-en-1-amine Formula: C₂₁H₂₁N Molecular Weight: 287.40 |
| 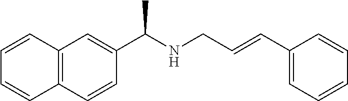 [SMILES: C[C@@H](NC/C=C/c1ccccc1)c1ccc2ccccc2c1] | Compound 35 Chemical Name: (R,E)-N- (1-(naphthalen-2-yl)ethyl)- 3-phenylprop-2-en-1-amine Formula: C₂₁H₂₁N Molecular Weight: 287.40 |
| 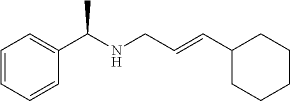 [SMILES: C[C@@H](NC/C=C/C1CCCCC1)c1ccccc1] | Compound 36 Chemical Name: (R,E)-3- cyclohexyl-N-(1- phenylethyl)prop-2-en-1- amine Formula: C₁₇H₂₅N Molecular Weight: 243.39 |
| 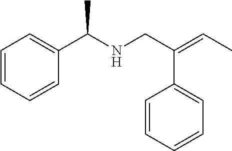 [SMILES: C/C=C(/CN[C@H](C)c1ccccc1)c1ccccc1] | Compound 37 Chemical Name: (R,E)-2- phenyl-N-(1- phenylethyl)but-2-en-1-amine Formula: C₁₈H₂₁N Molecular Weight: 251.37 |
| 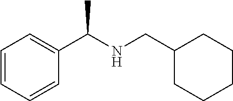 [SMILES: C[C@@H](NCC1CCCCC1)c1ccccc1] | Compound 38 Chemical Name: (R)-N- (cyclohexylmethyl)-1- phenylethanamine Formula: C₁₅H₂₃N Molecular Weight: 217.35 |
| 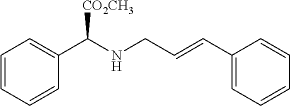 [SMILES: COC(=O)[C@@H](NC/C=C/c1ccccc1)c1ccccc1] | Compound 39 Chemical Name: (S)-methyl 2-(cinnamylamino)-2- phenylacetate Formula: C₁₈H₁₉NO₂ Molecular Weight: 281.35 |
| 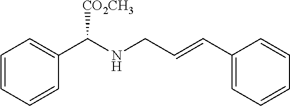 [SMILES: COC(=O)[C@H](NC/C=C/c1ccccc1)c1ccccc1] | Compound 40 Chemical Name: (R)-methyl 2-(cinnamylamino)-2- phenylacetate Formula: C₁₈H₁₉NO₂ Molecular Weight: 281.35 |
| 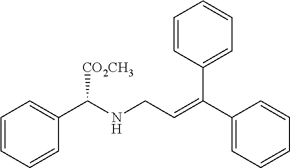 [SMILES: COC(=O)[C@H](NCC=C(c1ccccc1)c1ccccc1)c1ccccc1] | Compound 42 Chemical Name: (R)-methyl 2-((3,3-diphenylallyl)amino)- 2-phenylacetate Formula: C₂₄H₂₃NO₂ Molecular Weight: 357.44 |
| 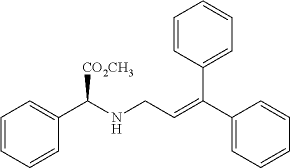 [SMILES: COC(=O)[C@@H](NCC=C(c1ccccc1)c1ccccc1)c1ccccc1] | Compound 43 Chemical Name: (S)-methyl 2-((3,3-diphenylallyl)amino)- 2-phenylacetate Formula: C₂₄H₂₃NO₂ Molecular Weight: 357.44 |
| 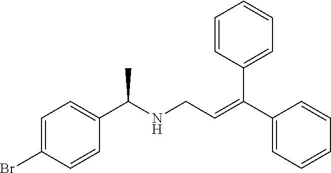 [SMILES: C[C@@H](NCC=C(c1ccccc1)c1ccccc1)c1ccc(Br)cc1] | Compound 44 Chemical Name: (R)-N-(1-(4- bromophenyl)ethyl)-3,3- diphenylprop-2-en-1-amine Formula: C₂₃H₂₃BrN Molecular Weight: 392.33 |
| 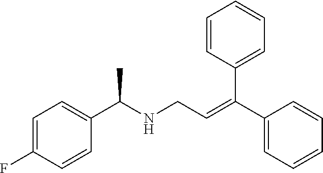 [SMILES: C[C@@H](NCC=C(c1ccccc1)c1ccccc1)c1ccc(F)cc1] | Compound 45 Chemical Name: (R)-N-(1-(4- fluorophenyl)ethyl)-3,3- diphenylprop-2-en-1-amine Formula: C₂₃H₂₂FN Molecular Weight: 331.43 |
| 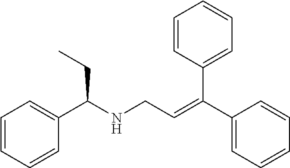 [SMILES: CC[C@@H](NCC=C(c1ccccc1)c1ccccc1)c1ccccc1] | Compound 46 Chemical Name: (R)-3,3- diphenyl-N-(1- phenylpropyl)prop-2-en-1- amine Formula: C₂₄H₂₅N Molecular Weight: 327.46 |
| 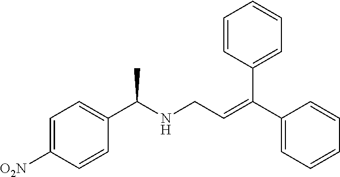 [SMILES: C[C@@H](NCC=C(c1ccccc1)c1ccccc1)c1ccc([N+](=O)[O-])cc1] | Compound 47 Chemical Name: (R)-4-(1- ((3,3- diphenylallyl)nitro)ethyl) aniline Formula: C₂₃H₂₂O₂N₂ Molecular Weight: 358.43 |
| 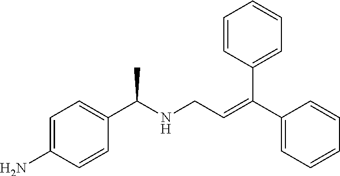 [SMILES: C[C@@H](NCC=C(c1ccccc1)c1ccccc1)c1ccc(N)cc1] | Compound 48 Chemical Name: (R)-4-(1- ((3,3- diphenylallyl)amino)ethyl) aniline Formula: C₂₃H₂₄N₂ Molecular Weight: 328.45 |
| 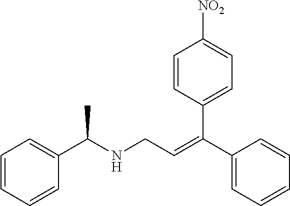 [SMILES: C[C@@H](NC/C=C(/c1ccccc1)c1ccc([N+](=O)[O-])cc1)c1ccccc1] | Compound 49 Chemical Name: (R,Z)-3- (4-nitrophenyl)-3-phenyl- N-(1-phenylethyl)prop-2- en-1-amine Formula: C₂₃H₂₂O₂N₂ Molecular Weight: 358.43 |
| 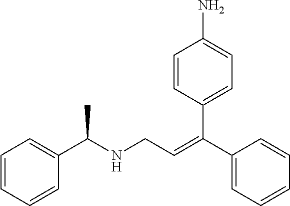 [SMILES: C[C@@H](NC/C=C(/c1ccccc1)c1ccc(N)cc1)c1ccccc1] | Compound 50 Chemical Name: (R,Z)-4-(1- phenyl-3-((1- phenylethyl)amino)prop-1- en-1-yl)aniline Formula: C₂₃H₂₄N₂ Molecular Weight: 328.45 |
| 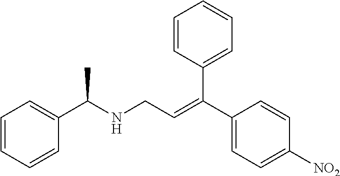 [SMILES: C[C@@H](NC/C=C(\c1ccccc1)c1ccc([N+](=O)[O-])cc1)c1ccccc1] | Compound 55 Chemical Name: (R,E)-3- (4-nitrophenyl)-3-phenyl- N-(1-phenylethyl)prop-2- en-1-amine Formula: C₂₃H₂₂N₂O₂ Molecular Weight: 358.43 |
| 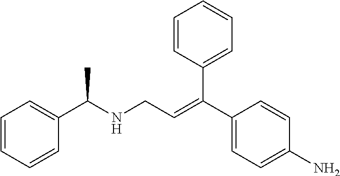 [SMILES: C[C@@H](NC/C=C(\c1ccccc1)c1ccc(N)cc1)c1ccccc1] | Compound 56 Chemical Name: (R,E)-4- (1-phenyl-3-((1- phenylethyl)amino)prop-1- en-1-yl)aniline Formula: C₂₃H₂₄N₂ Molecular Weight: 328.45 |
| 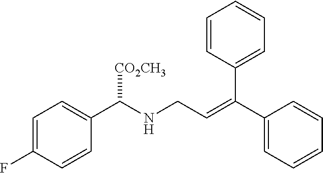 [SMILES: COC(=O)[C@H](NCC=C(c1ccccc1)c1ccccc1)c1ccc(F)cc1] | Compound 57 Chemical Name: (R)-methyl 2-((3,3- diphenylallyl)amino)-2-(4- fluorophenyl)acetate Formula: C₂₄H₂₂FNO₂ Molecular Weight: 375.44 |
| 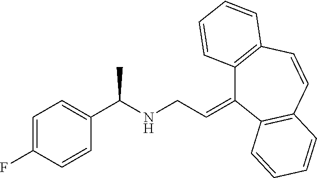 [SMILES: C[C@@H](NCC=C1c2ccccc2C=Cc2ccccc21)c1ccc(F)cc1] | Compound 58 Chemical Name: (R)-N-(2 (5Hdibenzo[a,d][7] annulen- 5-ylidene)ethyl)-1-(4- fluorophenyl)ethanamine Formula: C₂₅H₂₂FN Molecular Weight: 355.45 |
| 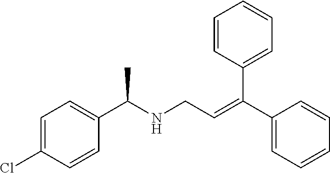 [SMILES: C[C@@H](NCC=C(c1ccccc1)c1ccccc1)c1ccc(Cl)cc1] | Compound 59 Chemical Name: (R)-N-(1- (4-chlorophenyl)ethyl)-3,3- diphenylprop-2-en-1-amine Formula: C₂₃H₂₂ClN Molecular Weight: 347.88 |
| 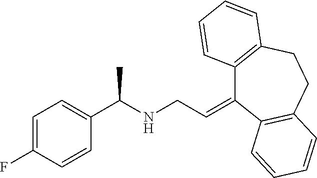 [SMILES: C[C@@H](NCC=C1c2ccccc2CCc2ccccc21)c1ccc(F)cc1] | Compound 60 Chemical Name: (R)-2- (10,11-dihydro-5H- dibenzo[a,d][7]annulen-5- ylidene)-N-(1-(4- fluorophenyl)ethyl) ethanamine Formula: C₂₅H₂₄FN Molecular Weight: 357.46 |
| 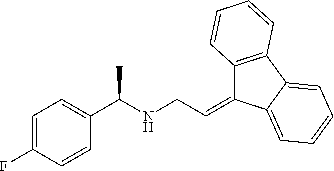 [SMILES: C[C@@H](NCC=C1c2ccccc2-c2ccccc21)c1ccc(F)cc1] | Compound 61 Chemical Name: (R)-N-(2- (9H-fluoren-9- ylidene)ethyl)-1-(4- fluorophenyl)ethanamine Formula: C₂₃H₂₀FN Molecular Weight: 329.41 |
| 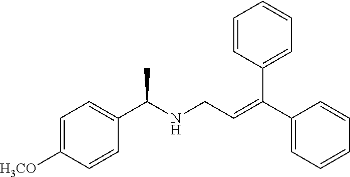 [SMILES: COc1ccc([C@@H](C)NCC=C(c2ccccc2)c2ccccc2)cc1] | Compound 62 Chemical Name: (R)-N-(1- (4-methoxyphenyl)ethyl)- 3,3-diphenylprop-2-en-1- amine Formula: C₂₄H₂₅NO Molecular Weight: 343.46 |
| 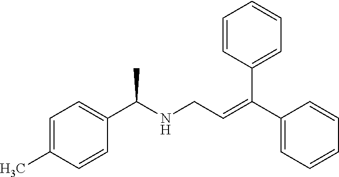 [SMILES: Cc1ccc([C@@H](C)NCC=C(c2ccccc2)c2ccccc2)cc1] | Compound 63 Chemical Name: (R)-3,3- diphenyl-N-(1-(p- tolyl)ethyl)prop-2-en-1- amine Formula: C₂₄H₂₅N Molecular Weight: 327.46 |
| 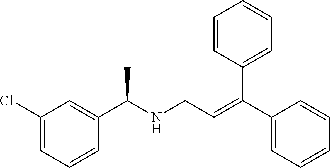 [SMILES: C[C@@H](NCC=C(c1ccccc1)c1ccccc1)c1cccc(Cl)c1] | Compound 64 Chemical Name: (R)-3,3- diphenyl-N-(1-(p- tolyl)ethyl)prop-2-en-1- amine Formula: C₂₃H₂₂ClN Molecular Weight: 347.88 |
| 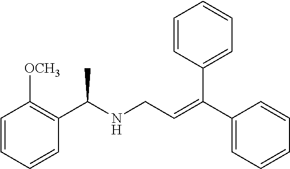 [SMILES: COc1ccccc1[C@@H](C)NCC=C(c1ccccc1)c1ccccc1] | Compound 65 Chemical Name: (R)-N-(1- (2-methoxyphenyl)ethyl)- 3,3-diphenylprop-2-en-1- amine Formula: C₂₄H₂₅NO Molecular Weight: 343.46 |
| 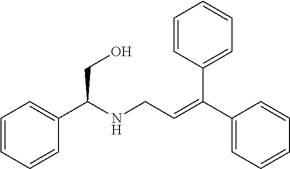 [SMILES: OC[C@@H](NCC=C(c1ccccc1)c1ccccc1)c1ccccc1] | Compound 66 Chemical Name: (S)-2-((3,3- diphenylallyl)amino)-2- phenylethanol Formula: C₂₃H₂₃NO Molecular Weight: 329.43 |
| 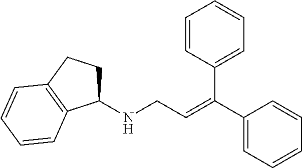 [SMILES: C(CN[C@@H]1CCc2ccccc21)=C(c1ccccc1)c1ccccc1] | Compound 67 Chemical Name: (R)-N-(3,3- diphenylallyl)-2,3-dihydro- 1H-inden-1-amine Formula: C₂₄H₂₃N Molecular Weight: 325.45 |
| 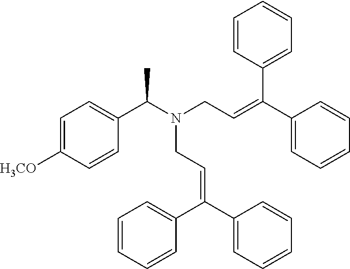 [SMILES: COc1ccc([C@@H](C)N(CC=C(c2ccccc2)c2ccccc2)CC=C(c2ccccc2)c2ccccc2)cc1] | Compound 68 Chemical Name: (R)-N-(3,3- diphenylallyl)-N-(1-(4- methoxyphenyl)ethyl)-3,3- diphenylprop-2-en-1-amine Formula: C₃₉H₃₇NO Molecular Weight: 535.72 |
| 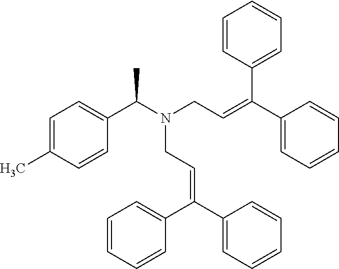 [SMILES: Cc1ccc([C@@H](C)N(CC=C(c2ccccc2)c2ccccc2)CC=C(c2ccccc2)c2ccccc2)cc1] | Compound 69 Chemical Name: (R)-N-(3,3- diphenylallyl)-3,3-diphenyl- N-(1-(p-tolyl)ethyl)prop-2- en-1-amine Formula: C₃₉H₃₇N Molecular Weight: 519.72 |
| 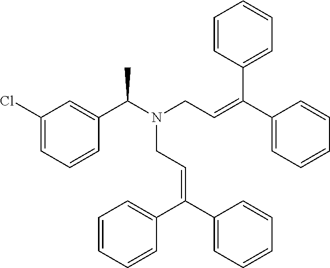 [SMILES: C[C@H](c1cccc(Cl)c1)N(CC=C(c1ccccc1)c1ccccc1)CC=C(c1ccccc1)c1ccccc1] | Compound 70 Chemical Name: (R)-N-(1- (3-chlorophenyl)ethyl)-N- (3,3-diphenylallyl)-3,3- diphenylprop-2-en-1-amine Formula: C₃₈H₃₄ClN Molecular Weight: 540.14 |
| 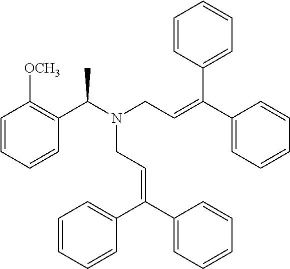 [SMILES: COc1ccccc1[C@@H](C)N(CC=C(c1ccccc1)c1ccccc1)CC=C(c1ccccc1)c1ccccc1] | Compound 71 Chemical Name: (S)-2- (bis(3,3- diphenylallyl)amino)-2- phenylethanol Formula: C₃₉H₃₇NO Molecular Weight: 535.72 |
| 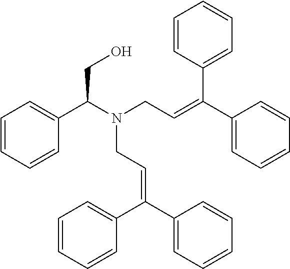 [SMILES: OC[C@H](c1ccccc1)N(CC=C(c1ccccc1)c1ccccc1)CC=C(c1ccccc1)c1ccccc1] | Compound 72 Chemical Name: (R)-N-(3,3- diphenylallyl)-N-(1-(2- methoxyphenyl)ethyl)-3,3- diphenylprop-2-en-1-amine Formula: C₃₈H₃₅NO Molecular Weight: 521.69 |
| 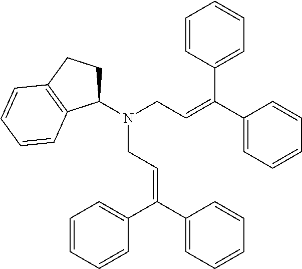 [SMILES: C(CN(CC=C(c1ccccc1)c1ccccc1)[C@@H]1CCc2ccccc21)=C(c1ccccc1)c1ccccc1] | Compound 73 Chemical Name: (R)-N,N- bis(3,3-diphenylallyl)-2,3- dihydro-1H-inden-1-amine Formula: C₃₉H₃₅N Molecular Weight: 517.70 |

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The compounds can be formulated as a mixture of one or more diastereomers. Alternatively, the diastereomers can be separated and one or more of the diastereomers can be formulated individually. The chiral centers of the compounds of the present invention can have the S or the R configuration, as defined by the IUPAC 1974 Recommendations. For example, mixtures of stereoisomers may be separated using the techniques taught in the Examples section below, as well as modifications thereof.

Atoms making up the compounds of the present invention are intended to include all isotopic forms of such atoms. Compounds of the present invention include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present invention include those that have been further modified to comprise substituents that are convertible to hydrogen in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

The compounds described herein may exist in unsolvated forms as well as solvated forms, including hydrated forms. In general, the solvated forms are equivalent to unsolvated forms and are within the scope of the compounds described herein. The compounds described herein may exist in multiple crystalline or amorphous forms. In general, all physical forms are equivalent for the uses described herein and are intended to be within the scope of the compounds described herein.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

### III. BIOLOGICAL ACTIVITY AND TREATMENT METHODS

The present disclosure describes fendiline derivatives for inhibition of K-ras localization to the plasma membrane. Such derivatives may be used for inhibiting the signal transduction from, among others, oncogenic K-ras. Treatment methods based on these derivatives may be used as therapies for cancer, including, for example, leukemia, colorectal cancer, pancreatic cancer and lung cancer.

Assay results for the mislocalization of K-ras localization to the plasma membrane and for the inhibition of ERK activation are shown for several compound of the present invention, as well as comparison compounds, in Table 2 below. To determine the effect of compounds on localization of K-ras, Mardine Darby canine kidney epithelial (MDCK) cells stably expressing GFP-tagged K-rasG12V (oncogenic K-ras) grown on cover slips were treated with vehicle or compound (4 μM) for 48 hours and fixed with 4% paraformaldehyde. The coverslips were mounted in Mowiol and imaged by confocal microscopy (Nikon A1) using a 60× objective. Images were analyzed by ImageJ software. Densitometric analysis was performed on images to determine the degree of K-ras mislocalization. To determine the effect of compounds on K-ras function, MDCK cells stably expressing GFP-tagged K-rasG12V were treated with vehicle or compound (4 μM) for 48 hours. Following treatment, whole cell lysates were prepared and subjected SDS-PAGE and western blotting to quantify the level of phosphorylated ERK, a protein activated downstream of K-ras. All calculations were normalized to vehicle-treated control.

TABLE 2
Fendiline Derivatives and their Effect on K-ras Localization and Function.

| | | % | % Inhibition |
|---|---|---|---|
| | | Mislocalization | of ERK |
| | | of K-ras when | activation |
| | | treated with | when treated |
| | | 4 μM | with 4 μM |
| Compound | Formula | compound | compound |
| DMSO | (CH₃)₂SO | 0 | 0 |
| (vehicle) | | | |
| Fendiline | 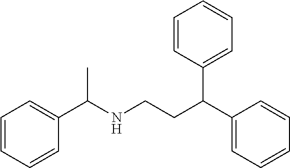 [SMILES: CC(NCCC(c1ccccc1)c1ccccc1)c1ccccc1] | 20.53 | 7.37 |
| 02 | 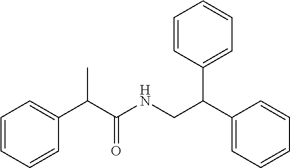 [SMILES: CC(C(=O)NCC(c1ccccc1)c1ccccc1)c1ccccc1] | 13.25 | NT |
| 03 | 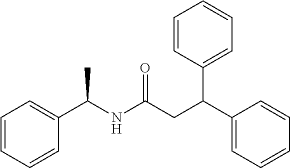 [SMILES: C[C@@H](NC(=O)CC(c1ccccc1)c1ccccc1)c1ccccc1] | 25.84 | NT |
| 04 | 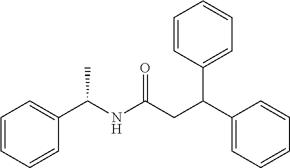 [SMILES: C[C@H](NC(=O)CC(c1ccccc1)c1ccccc1)c1ccccc1] | 9.73 | NT |
| 05 | 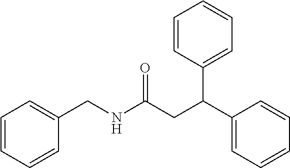 [SMILES: O=C(CC(c1ccccc1)c1ccccc1)NCc1ccccc1] | 4.01 | NT |
| 06 | 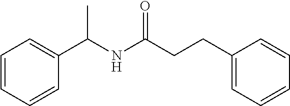 [SMILES: CC(NC(=O)CCc1ccccc1)c1ccccc1] | 28.89 | NT |
| 07 | 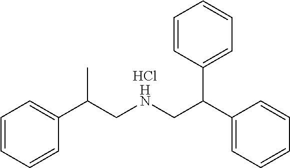 [SMILES: CC(CNCC(c1ccccc1)c1ccccc1)c1ccccc1 ; Cl] | 21.93 | NT |
| 08 (R- Fendiline) | 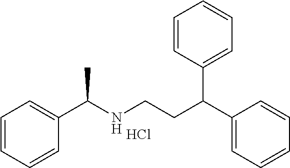 [SMILES: C[C@@H](NCCC(c1ccccc1)c1ccccc1)c1ccccc1 ; Cl] | 36.05 | 38.23 |
| 09 (S- Fendiline) | 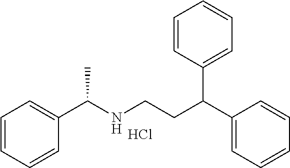 [SMILES: C[C@H](NCCC(c1ccccc1)c1ccccc1)c1ccccc1 ; Cl] | 20.41 | NT |
| 10 | 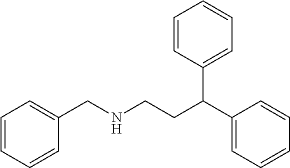 [SMILES: c1ccc(CNCCC(c2ccccc2)c2ccccc2)cc1] | 20.22 | NT |
| 11 | 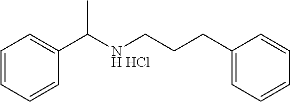 [SMILES: CC(NCCCc1ccccc1)c1ccccc1 ; Cl] | 32.42 | 42.94 |
| 12 | 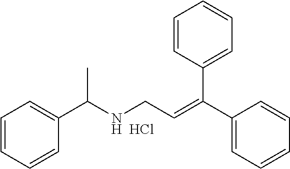 [SMILES: CC(NCC=C(c1ccccc1)c1ccccc1)c1ccccc1 ; Cl] | 37.70 | 55.09 |
| 13 | 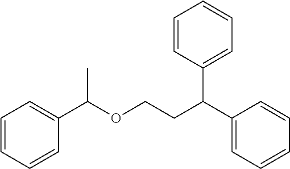 [SMILES: CC(OCCC(c1ccccc1)c1ccccc1)c1ccccc1] | 31.96 | 28.89 |
| 14 | 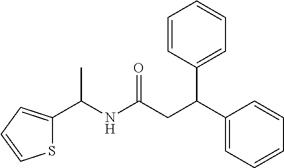 [SMILES: CC(NC(=O)CC(c1ccccc1)c1ccccc1)c1cccs1] | 23.27 | NT |
| 15 | 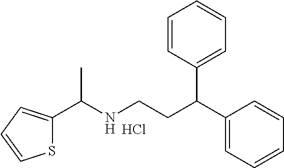 [SMILES: CC(NCCC(c1ccccc1)c1ccccc1)c1cccs1 ; Cl] | 25.08 | NT |
| 16 | 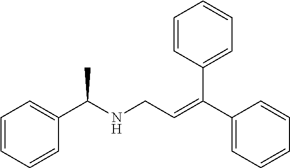 [SMILES: C[C@@H](NCC=C(c1ccccc1)c1ccccc1)c1ccccc1] | 39.30 | 58.36 |
| 17 | 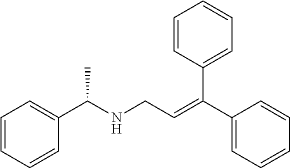 [SMILES: C[C@H](NCC=C(c1ccccc1)c1ccccc1)c1ccccc1] | 8.27 | −51.85 |
| 18 | 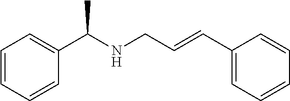 [SMILES: C[C@@H](NC/C=C/c1ccccc1)c1ccccc1] | 17.55 | −2.54 |
| 19 | 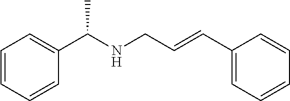 [SMILES: C[C@H](NC/C=C/c1ccccc1)c1ccccc1] | 16.47 | 11.29 |
| 20 | 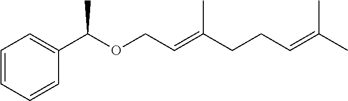 [SMILES: CC(C)=CCC/C(C)=C/CO[C@H](C)c1ccccc1] | 14.59 | −5.12 |
| 21 | 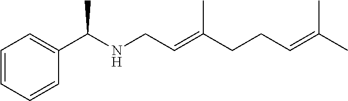 [SMILES: CC(C)=CCC/C(C)=C/CN[C@H](C)c1ccccc1] | 8.29 | 3.75 |
| 22 | 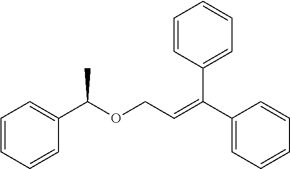 [SMILES: C[C@@H](OCC=C(c1ccccc1)c1ccccc1)c1ccccc1] | 8.14 | −14.38 |
| 23 | 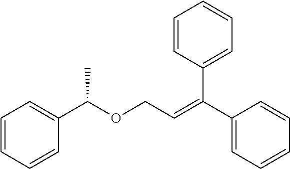 [SMILES: C[C@H](OCC=C(c1ccccc1)c1ccccc1)c1ccccc1] | 10.32 | −0.95 |
| 24 | 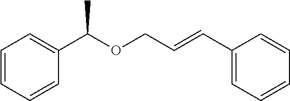 [SMILES: C[C@@H](OC/C=C/c1ccccc1)c1ccccc1] | 17.96 | 7.19 |
| 25 | 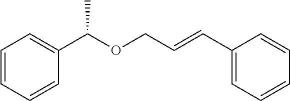 [SMILES: C[C@H](OC/C=C/c1ccccc1)c1ccccc1] | 33.94 | 56.82 |
| 26 | 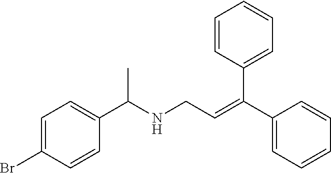 [SMILES: CC(NCC=C(c1ccccc1)c1ccccc1)c1ccc(Br)cc1] | 44.80 | 43.48 |
| 27 | 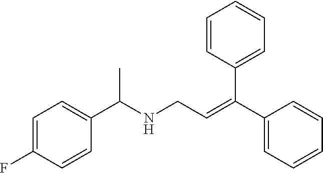 [SMILES: CC(NCC=C(c1ccccc1)c1ccccc1)c1ccc(F)cc1] | 45.22 | 49.33 |
| 28 | 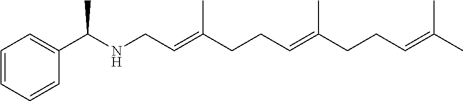 [SMILES: CC(C)=CCC/C(C)=C/CC/C(C)=C/CN[C@H](C)c1ccccc1] | 34.87 | 38.03 |
| 29 | 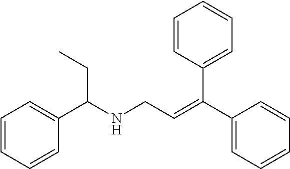 [SMILES: CCC(NCC=C(c1ccccc1)c1ccccc1)c1ccccc1] | 37.04 | 40.32 |
| 30 | 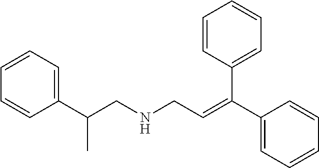 [SMILES: CC(CNCC=C(c1ccccc1)c1ccccc1)c1ccccc1] | 26.49 | 49.06 |
| 31 | 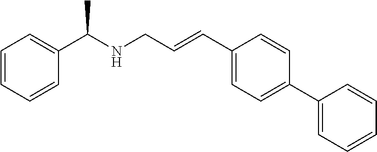 [SMILES: C[C@@H](NC/C=C/c1ccc(-c2ccccc2)cc1)c1ccccc1] | 6.36 | 35.64 |
| 32 | 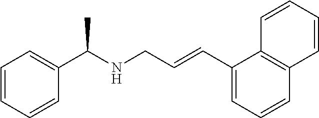 [SMILES: C[C@@H](NC/C=C/c1cccc2ccccc12)c1ccccc1] | 12.52 | 31.71 |
| 33 | 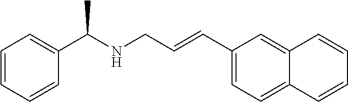 [SMILES: C[C@@H](NC/C=C/c1ccc2ccccc2c1)c1ccccc1] | 1.53 | 15.68 |
| 34 | 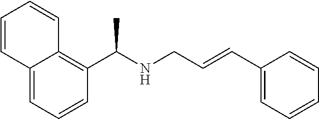 [SMILES: C[C@@H](NC/C=C/c1ccccc1)c1cccc2ccccc12] | 7.94 | 18.94 |
| 35 | 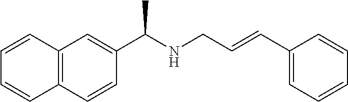 [SMILES: C[C@@H](NC/C=C/c1ccccc1)c1ccc2ccccc2c1] | 26.05 | −21.56 |
| 37 | 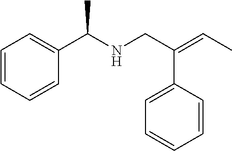 [SMILES: C/C=C(/CN[C@H](C)c1ccccc1)c1ccccc1] | 6.82 | 18.94 |
| 38 | 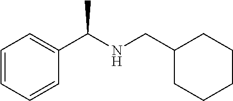 [SMILES: C[C@@H](NCC1CCCCC1)c1ccccc1] | 9.67 | 32.72 |
| 39 | 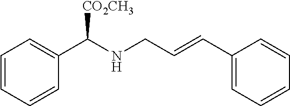 [SMILES: COC(=O)[C@@H](NC/C=C/c1ccccc1)c1ccccc1] | 6.72 | −10.24 |
| 40 | 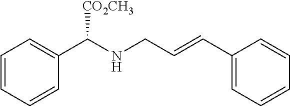 [SMILES: COC(=O)[C@H](NC/C=C/c1ccccc1)c1ccccc1] | 9.46 | 11.03 |
| 42 | 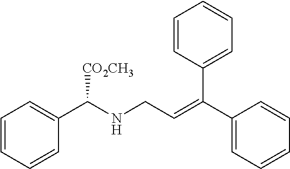 [SMILES: COC(=O)[C@H](NCC=C(c1ccccc1)c1ccccc1)c1ccccc1] | 50.00 | 43.99 |
| 43 | 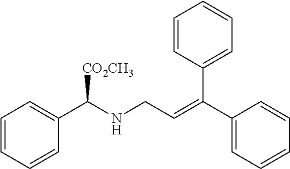 [SMILES: COC(=O)[C@@H](NCC=C(c1ccccc1)c1ccccc1)c1ccccc1] | 30.15 | 38.39 |
| 44 | 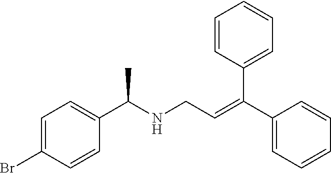 [SMILES: C[C@@H](NCC=C(c1ccccc1)c1ccccc1)c1ccc(Br)cc1] | 38.13 | 38.10 |
| 45 | 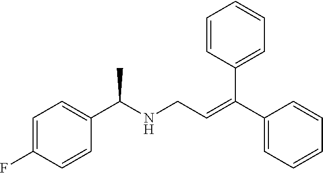 [SMILES: C[C@@H](NCC=C(c1ccccc1)c1ccccc1)c1ccc(F)cc1] | 50.00 | 43.47 |
| 46 | 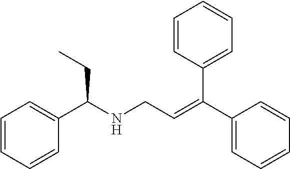 [SMILES: CC[C@@H](NCC=C(c1ccccc1)c1ccccc1)c1ccccc1] | 37.53 | 53.97 |
| 47 | 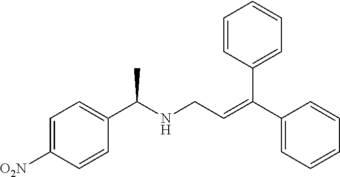 [SMILES: C[C@@H](NCC=C(c1ccccc1)c1ccccc1)c1ccc([N+](=O)[O-])cc1] | 34.03 | 70.58 |
| 48 | 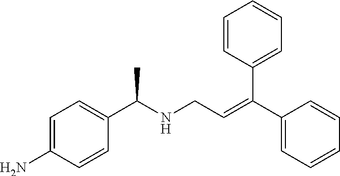 [SMILES: C[C@@H](NCC=C(c1ccccc1)c1ccccc1)c1ccc(N)cc1] | 28.18 | 53.45 |
| 49 | 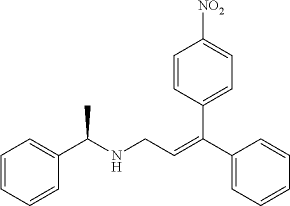 [SMILES: C[C@@H](NC/C=C(/c1ccccc1)c1ccc([N+](=O)[O-])cc1)c1ccccc1] | 20.13 | 36.75 |
| 50 | 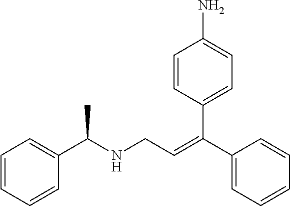 [SMILES: C[C@@H](NC/C=C(/c1ccccc1)c1ccc(N)cc1)c1ccccc1] | 6.61 | 22.55 |
| 55 | 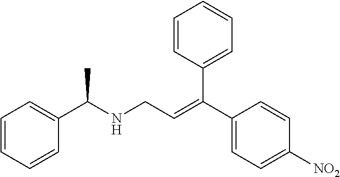 [SMILES: C[C@@H](NC/C=C(\c1ccccc1)c1ccc([N+](=O)[O-])cc1)c1ccccc1] | 12.29 | −36.6 |
| 56 | 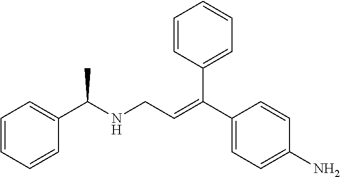 [SMILES: C[C@@H](NC/C=C(\c1ccccc1)c1ccc(N)cc1)c1ccccc1] | 17.27 | −191 |
| 57 | 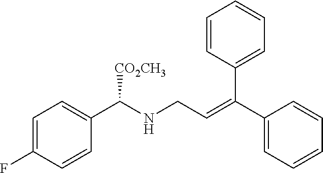 [SMILES: COC(=O)[C@H](NCC=C(c1ccccc1)c1ccccc1)c1ccc(F)cc1] | 12.66 | −105 |
| 58 | 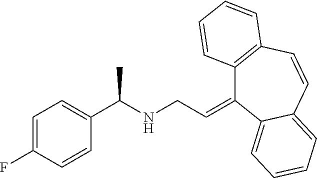 [SMILES: C[C@@H](NCC=C1c2ccccc2C=Cc2ccccc21)c1ccc(F)cc1] | 5.05 | −78.7 |
| 59 | 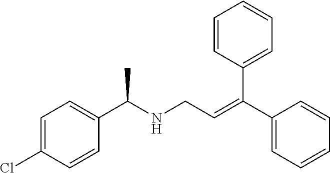 [SMILES: C[C@@H](NCC=C(c1ccccc1)c1ccccc1)c1ccc(Cl)cc1] | NT | NT |
| 60 | 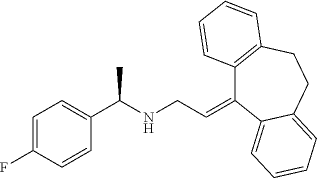 [SMILES: C[C@@H](NCC=C1c2ccccc2CCc2ccccc21)c1ccc(F)cc1] | NT | NT |

NT = not tested.

It has been demonstrated in some embodiments that the fendiline derivatives thereof significantly and specifically inhibit normal K-ras association with the plasma membrane. Such inhibition may be used to inhibit signaling downstream of this protein, which may in turn be used to treat K-ras mediated disorders, such as cancer, including, for example, leukemia, colon cancer, pancreatic cancer and lung cancer.

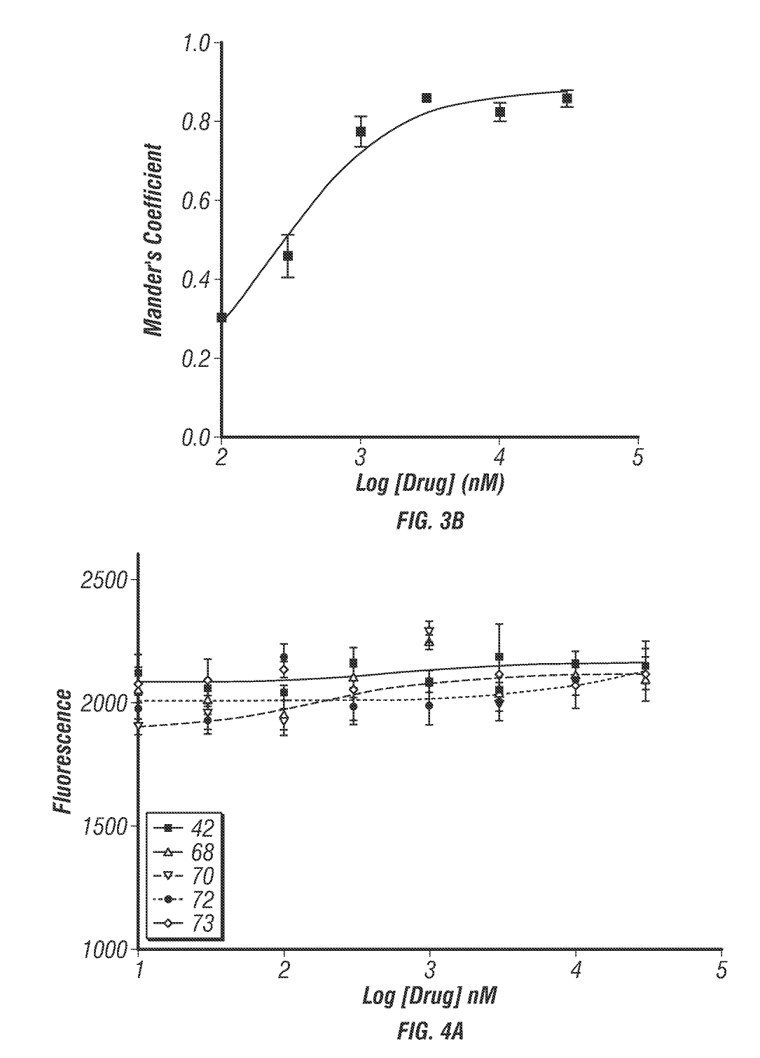

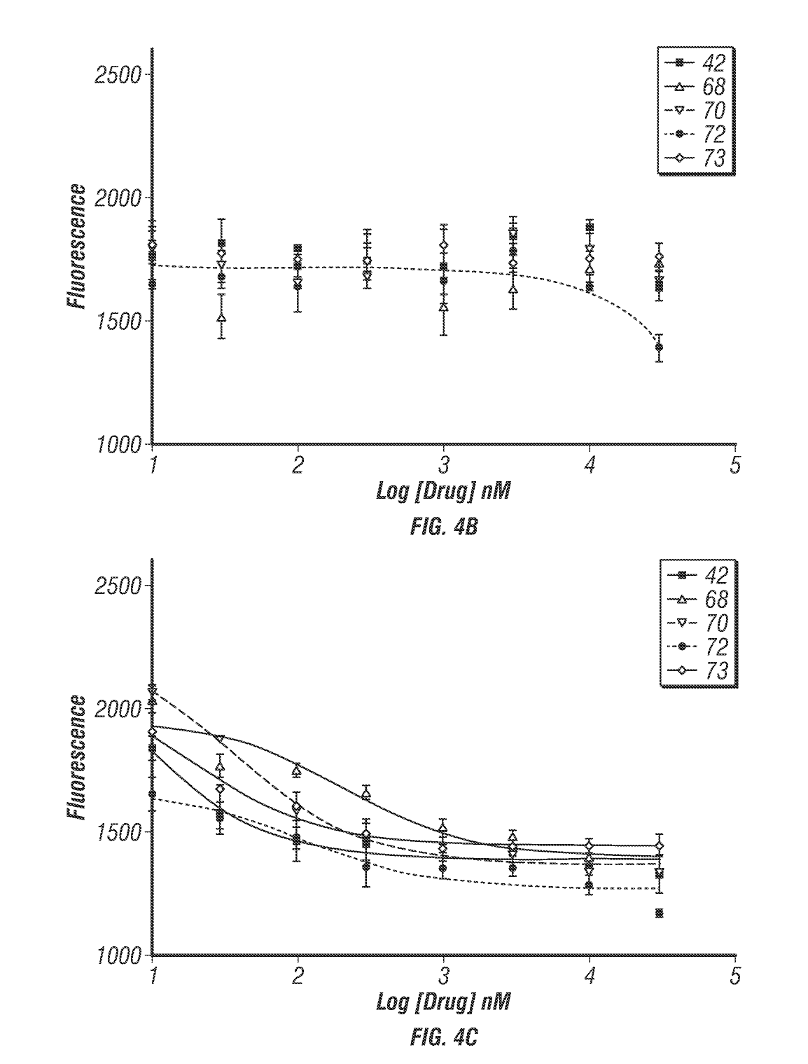

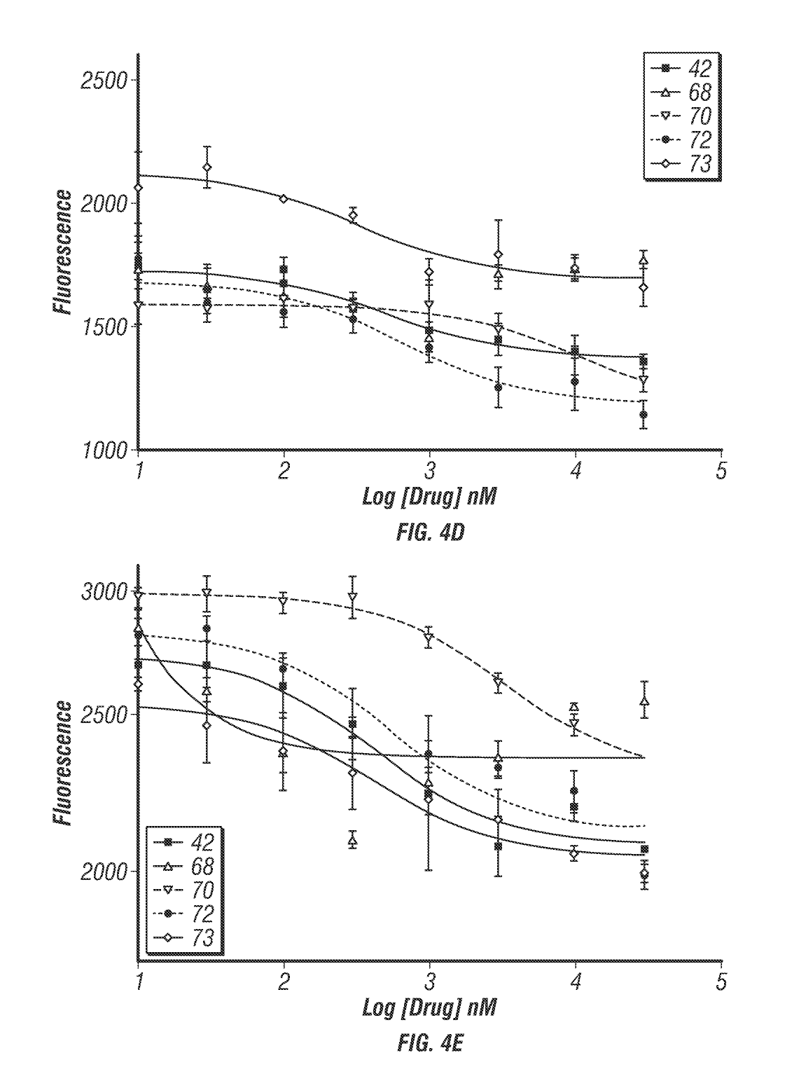

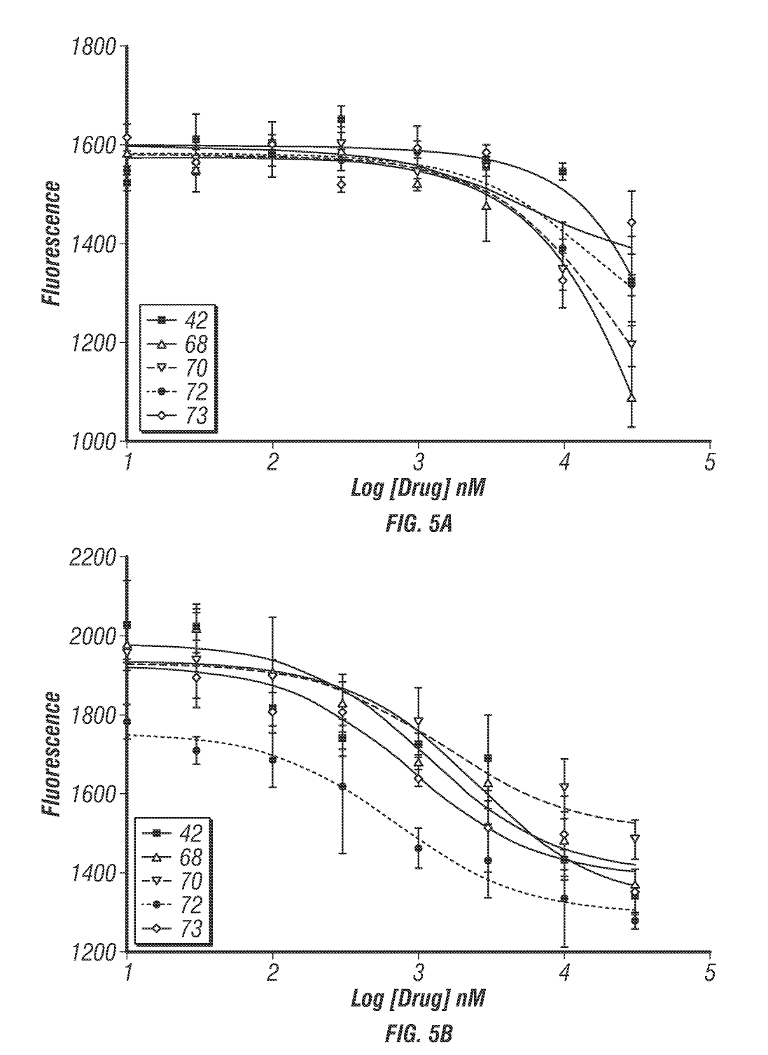

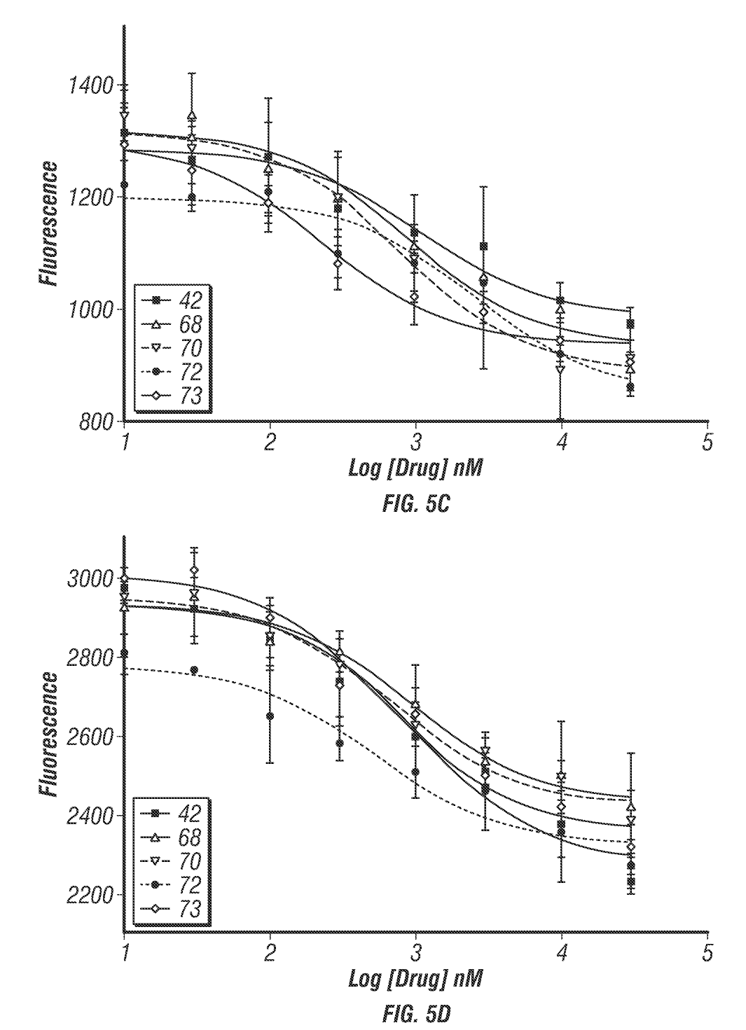

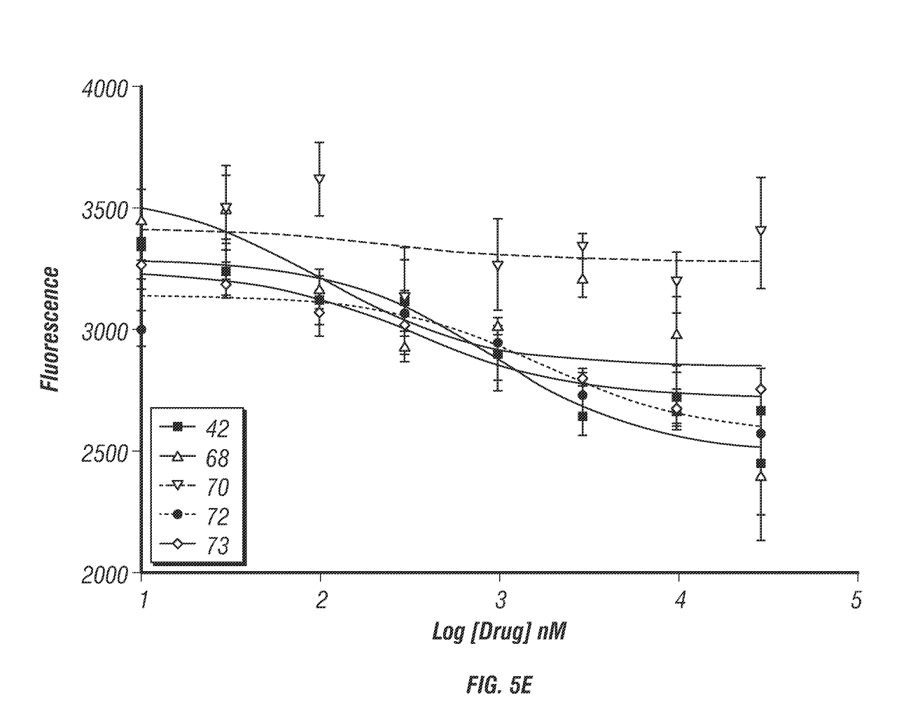

Furthermore, in some embodiments, it has been determined that the different fendiline derivatives have specific EC₅₀ values for their ability to induce the K-Ras mislocalization. These EC₅₀ along with along with the value for the maximal effects elicited by the compounds and the cell viability 48 hours after treatment may be as shown in Table 3. Concentrations of compounds that give half of the maximal effect (EC₅₀), maximal effects elicited by the compounds (Eₘₐₓ) and viability of cells after 48 h treatment with 30 μM compound. EC₅₀ values were derived from the concentration response curves of compounds for K-Ras mislocalization (example shown in FIGS. 4 and 5). Based on the results shown in the tables, in some embodiments, some of the compounds showed potentially promising characteristics as a drug candidate including but not limited to high potency (ie., low EC₅₀) and high efficacy (ie., high Eₘₐₓ) with little or no cytotoxic effect at 30 μM. Compounds showing promising characteristics may be subjected to additional testing including the proliferation assay.

TABLE 3
Concentrations of compounds that give half of the
maximal effect (EC₅₀), maximal effects elicited by
the compounds (Eₘₐₓ) and viability of cells after
48 hour treatment with 30 μM compound.

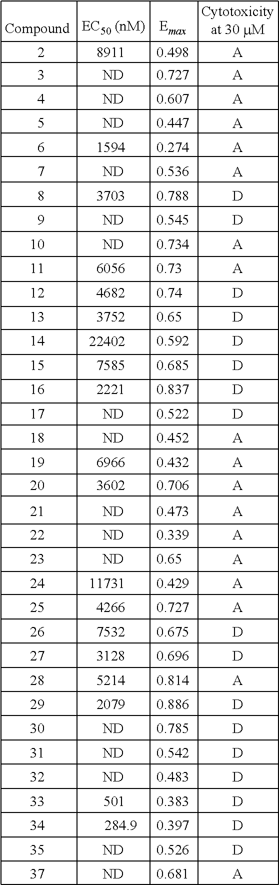

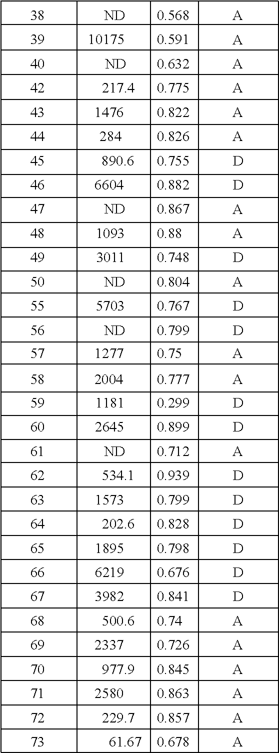

ND—EC₅₀ not determined within the dose range tested
A—cells alive -or- D—cells dead
Rows shaded in dark gray were tested in the proliferation assay.

The Systematic (IUPAC) name for fendiline is 3,3-diphenyl-N-(1-phenylethyl)propan-1-amine (CAS number 13042-18-7, ATC code C08EA01, PubChem CID 3336). The CAS number for fendiline hydrochloride is 13042-18-5. It has been reported that fendiline significantly and specifically alters the localization of K-ras protein and inhibits signaling downstream of this protein, possibly as a non-selective L-type Ca²⁻ channel blocker. Fendiline has also been reported to function as a calmodulin antagonist. See, for example, Lückhoff, et al., 1991, which is incorporated herein by reference.

It has been demonstrated that the function of fendiline as a K-ras inhibitor is unrelated to changes in intracellular Ca²⁺, or blockade of Ca²⁺ channels, a mode of action with which it has been previously associated. As a Ca²⁺ channel blocker it has been used as a vasodilator for the treatment of ischemic heart disease, where it is applied at 150 mg daily in divided doses taken orally. Therefore a good deal of the pharmacokinetics of fendiline administered to humans both orally (single and in escalating multiple dose regimens) and by intravenous administration (single 3 mg dose), among others have been reported. See for example, Kukovetz, et al., 1982; Weyhenmeyer, et al., 1987, both of which are incorporated herein by reference. The plasma concentration of fendiline that results in inhibition of localization of K-ras and thus inhibits signaling that normally occurs downstream K-ras occurs at or about between 1 and 15 μM. In some embodiments, it occurs at 1, 2, 3, 4, 5, 6, 7, 8, 9, 10, 11, 12, 13, 14 or 15 μM.

In some embodiments, the fendiline derivatives provided herein may be used to significantly and/or specifically alter the localization of K-ras and thus inhibit signaling that normally occurs downstream of K-ras. In some embodiments, the fendiline derivatives provided herein may be used to disrupt K-ras signaling in a manner that is unrelated to changes in intracellular Ca²⁺, or blockade of Ca²⁺ channels.

In some embodiments, the fendiline derivatives provided herein may be used to inhibit the proliferation of endometrial cancer cells expressing oncogenic K-ras in a manner that is mediated by inhibition of K-ras translocation and signaling.

In some embodiments, there are provided methods of inhibiting K-ras signaling by applying one or more of the fendiline derivatives provided herein in a manner that blocks the association of K-ras protein with the plasma membrane. In some embodiments, there are provided methods of inhibiting K-ras signaling by applying one or more of the fendiline derivatives provided herein in a manner that inhibits the localization of K-ras protein to the plasma membrane. In some embodiments, there are provided methods of inhibiting K-ras signaling by applying one or more of the fendiline derivatives provided herein in a manner that inhibits the delivery of K-ras protein to the plasma membrane. In some embodiments, there are provided methods of inhibiting K-ras signaling by applying one or more of the fendiline derivatives provided herein in a manner that displacing K-ras protein from the plasma membrane. In some embodiments, there are provided methods of inhibiting K-ras signaling by applying one or more of the fendiline derivatives provided herein in a manner that blocks the association of K-ras protein with the plasma membrane. In some embodiments, the therapeutically effective amount of the fendiline derivatives in plasma is between 1 and 15 μM. In some embodiments, the patient is a mammal In some embodiments, the patient is a primate. In some embodiments, the patient is a companion animal. In some embodiments, the patient is human.

In other embodiments, there is a method of identifying a compound that blocks the association of K-ras protein with the plasma membrane. In other embodiments, there is a method of identifying a compound that inhibits K-ras signaling. In other embodiments, there is a method of identifying a compound that blocks the association of K-ras protein with the plasma membrane. In other embodiments, there is a method of identifying a compound that prevents the localization of K-ras to the plasma membrane. In other embodiments, there is a method of identifying a compound that prevents the delivery of K-ras to the plasma membrane. In other embodiments, there is a method of identifying a compound that directly inhibits K-ras plasma membrane interactions. In other embodiments, there is a method of identifying a compound that displaces K-ras protein from the plasma membrane.

Compounds that inhibit the association of K-ras with the plasma membrane, such as the fendiline derivative provided herein, may be used as therapeutics for one or more of the diseases discussed herein.

The therapeutically effective doses are readily determinable using an animal model, as described in U.S. Provisional Application No. 61/640,451, which is incorporated herein by reference. For example, a good deal of the pharmacokinetics of fendiline administered to humans both orally (single and in escalating multiple dose regimens) and by intravenous administration (single 3 mg dose), among others have been reported (see for example, Kukovetz, et al., 1982; Weyhenmeyer, et al., 1987, both of which are incorporated herein by reference. The concentration of fendiline that results in inhibition of localization of K-ras and thus inhibits signaling that normally occurs downstream K-ras occurs at or about between 0.01 and 15 μM (for example, but not limited to, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14 or 15 μM).

For example, experimental animals bearing solid tumors are frequently used to optimize appropriate therapeutic doses prior to translating to a clinical environment. Such models are known to be reliable in predicting effective anti-cancer strategies. Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, expressed as the ratio LD₅₀/ED₅₀. Compositions that exhibit large therapeutic indices are preferred. Compounds that exhibit toxic side effects may be used in certain embodiments, however, care should usually be taken to design delivery systems that target such compositions preferentially to the site of affected tissue, in order to minimize potential damage to uninfected cells and, thereby, reduce side effects.

In certain embodiments, it may be desirable to provide continuous delivery of one or more fendiline derivatives to a patient in need thereof. For intravenous or intraarterial routes, this can be accomplished using drip systems, such as by intravenous administration. For topical applications, repeated application can be done or a patch can be used to provide continuous administration of the fendiline derivatives over an extended period of time. Extended release formulations can also be used that provide limited but constant amounts of the drug over an extended period of time.

For internal applications, continuous perfusion of the region of interest may be desirable. This could be accomplished by catheterization, post-operatively in some cases, followed by continuous administration of the one or more fendiline derivatives. The time period for perfusion can be readily determined by the attending physician clinician for a particular patient. Perfusion times typically range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered.

The compositions described herein contain an effective amount of the one or more fendiline derivatives. The amount to be administered can be readily determined by the attending physician based on a variety of factors including, but not limited to, age of the patient, weight of the patient, disease or disorder to be treated, presence of a pre-existing condition, and dosage form to be administered (e g., immediate release versus modified release dosage form). Typically, the effective amount is from about 0.1 mg/kg/day to about 100 mg/kg/day, more preferably from 0.1 mg/kg/day to 50 mg/kg/day, more preferably from 0.1 mg/kg/day to 25 mg/kg/day, and most preferably from 0.1 mg/kg/day to 10 mg/kg/day. Dosages greater or less than this may be administered depending on the diseases or disorder to be treated. For example, preliminary data suggests that fendilines, such as Fendiline, are effective at inhibiting chemotaxis at picomolar and nanomolar concentrations, as discussed above.

A. Treatment of Cancer

In some embodiments, the fendiline derivatives described herein may be administered to a subject in need thereof to treat the subject either prophylactically (i.e., to prevent cancer) or therapeutically (i.e., to treat cancer after it has been detected), including reducing tumor growth, reducing the risk of local invasiveness of a tumor, increasing survival time of the patient, and/or reducing the risk of metastasis of a primary tumor. The compounds described herein can contact a target cell to inhibit the initiation and promotion of cancer, to kill cancer/malignant cells, to inhibit cell growth, to induce apoptosis, to inhibit metastasis, to decrease tumor size, to otherwise reverse or reduce the malignant phenotype of tumor cells, and combinations thereof. This may be achieved by contacting a tumor or tumor cell with a single composition or pharmacological formulation that includes the fendiline derivative(s), or by contacting a tumor or tumor cell with more than one distinct composition or formulation, simultaneously, wherein one composition includes one or more fendiline derivatives described herein and the other includes a second agent.

Exemplary cancers which may be treated include, but are not limited to, cancer of the skin, colon, uterine, ovarian, pancreatic, lung, bladder, breast, renal system, and prostate. Other cancers include, but are not limited to, cancers of the brain, liver, stomach, esophagus, head and neck, testicles, cervix, lymphatic system, larynx, esophagus, parotid, biliary tract, rectum, endometrium, kidney, and thyroid; including squamous cell carcinomas, adenocarcinomas, small cell carcinomas, gliomas, neuroblastomas, and the like. Assay methods for ascertaining the relative efficacy of the compounds described herein in treating the above types of cancers as well as other cancers are well known in the art.

In some embodiments, the compounds described herein may also be used to treat metastatic cancer either in patients who have received prior chemo, radio, or biological therapy or in previously untreated patients. In some embodiments, the patient will have received previous chemotherapy. Patients can be treated using a variety of routes of administration including systemic administration, such as intravenous administration or subcutaneous administration, oral administration or by intratumoral injection. In some embodiments, a pharmaceutical dose(s) may be administered that contains between 10 and 25 mg of a fendiline derivative provided herein per kg of patient body weight per day, including about 13, 16, 19, and 22 mg/kg/day. Alternatively, the patient could be treated with one or more pharmaceutical compositions comprising from about 1 mg/kg/day of a fendiline derivative provided herein to about 100 mg/kg/day, including about 3, 6, 9, 12, 15, 18, 21, 28, 30, 40, 50, 60, 70, 80 and 90 mg/kg/day of a fendiline derivative provided herein.

The treatment course typically consists of daily treatment for a minimum of eight weeks or one injection weekly for a minimum of eight weeks. Upon election by the clinician, the regimen may be continued on the same schedule until the tumor progresses or the lack of response is observed.

In some embodiments, the fendiline derivatives described herein can also be used to treat patients who have been rendered free of clinical disease by surgery, chemotherapy, and/or radiotherapy. In these aspects, the purpose of therapy is to prevent or reduce the likelihood of recurrent disease. Adjuvant therapy can be administered in the same regimen as described above to prevent recurrent disease.

In some embodiments, the fendiline derivatives described herein can also be used to target and/or kill cancer stem cells (CSCs).

### IV. FORMULATIONS

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

A. Parenteral Formulations

The compounds described herein can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium salts of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In one embodiment, the fendiline derivative(s) are formulated in a carrier containing 5% dextrose, alone or in combination with 10% propylene glycol. In another embodiment, the fendilines are formulated in 150 mM NaCl solution and 10 mM sodium acetate (pH adjusted to 4.5), optionally containing polysorbate 80. Formulations may be stable over a period of 6 months when stored at room temperature or 5° C., with the fendiline purity averaging about 95%.

B. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition which may include plasticizers, pigments, colorants, stabilizing agents, and glidants. Delayed release dosage formulations may be prepared as described in standard references such as “Pharmaceutical dosage form tablets” (1989), “Remington—The science and practice of pharmacy” (2000), and “Pharmaceutical dosage forms and drug delivery systems” (1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more fendiline derivatives and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In some embodiments, one or more fendiline derivatives described herein and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still other embodiments, one or more fendiline derivatives, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the fendilines and/or additional active agents.

### V. KITS

In various aspects, a kit is envisioned containing one or more compounds described herein. The kit may contain one or more sealed containers, such as a vial, containing any of the compounds described herein and/or reagents for preparing any of the compounds described herein. In some embodiments, the kit may also contain a suitable container means, which is a container that will not react with components of the kit, such as an Eppendorf tube, an assay plate, a syringe, a bottle, or a tube. The container may be made from sterilizable materials such as plastic or glass.

The kit may further include instructions that outline the procedural steps for methods of treatment or prevention of disease, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of one or more compounds described herein.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the claims.

### Methods and Materials

Cell lines: Madine Darby Canine Kidney epithelial cells (MDCK) stably co-expressing mGFP-tagged full length oncogenic mutant K-Ras (GFP-K-RasG12V) and mCherry-tagged to the amino acids 179-189 of H-RasC181S, C184S (mCherry-CAAX, localizes primarily to endomembranes) were grown in DMEM-high glucose/sodium pyruvate/10% FBS. The medium was supplemented with 1× Penicillin/Streptomycin for fluorescence microscopy assays, to avoid any microbial contamination from the test compounds added. KLE and Hec50 cells were maintained in DMEM-F-12 medium supplemented with 10% FBS. Hec-1A and Hec-1B cells were grown in McCoy's 5a medium supplemented with 10% FBS. ESS-1 cells were grown in RPMI 1640 medium supplemented with 20% FBS. MPanc96 cells were grown in DMEM supplemented with 10% FBS, MiaPaCa-2 cells in DMEM supplemented with 10% FBS and 2.5% horse serum and all other cell lines were grown in RPMI 1640 supplemented with 10% FBS. All cancer cell media were supplemented with 1× Penicillin/Streptomycin. All cell lines were grown at 37° C. with 5% CO₂.

Fluorescence microscopy: 2×10⁵ or 1×10⁵ cells/well were seeded in 6- or 12-well cell culture plates, respectively, containing glass coverslips. 24 h after seeding, fresh medium containing 1% vehicle (Dimethyl sulfoxide, DMSO) or 4 μM test compound (1:100 dilution, such that DMSO was 1%) was added and incubated for 48 h, following which the cells were fixed with 4% paraformaldehyde and coverslips were mounted in Mowiol. Cells were imaged in the GFP wavelength (488 nm) using a confocal microscope (Nikon A1) with 60× objective. Images were saved in tagged imaged file format (tiff) and analyzed by ImageJ software. The densitometric ratio of plasma membrane localized K-Ras versus total K-Ras was calculated and normalized to vehicle-treated control.

Western Immunoblotting for pERK: 2×10⁵ cells/well were seeded in 6-well cell culture plates. 24 h after seeding, fresh medium containing 1% vehicle (DMSO) or 4 μM test compound (1:100 dilution, such that DMSO was 1%) was added and incubated for 48 h. Cells were lysed in Buffer B (50 mM Tris [pH 7.5], 75 mM NaCl, 25 mM NaF, 5 mM MgCl₂, 5 mM EGTA, 1 mM dithiothreitol, 100 μM NaVO₄, 1% Nonidet P-40 plus protease inhibitors). SDS-PAGE and immunoblotting with the anti-phospho-p44/42 mitogen-activated protein kinase (MAPK) (ERK1/2) antibody was performed using 20 μg of each whole cell lysate. Signal was detected by enhanced chemiluminescence (SuperSignal; Pierce, Thermo Fisher Scientific, Rockford, Ill.) and imaged by FluorChemQ (Alpha Inotech, San Leandro, Calif.). Quantification of intensities was performed using FluorChemQ software. Results were normalized to vehicle-treated control.

K-Ras Mislocalization Assay: MDCK co-expressing GFP-K-rasG12V and mCherry-CAAX were grown on coverslips, treated with 0.1% vehicle (DMSO) or various concentrations of drugs for 48 h, and fixed with 4% paraformaldehyde. The coverslips were mounted in mowiol and imaged by confocal microscopy (Nikon A1) using a 60× objective. Using ImageJ software v1.42q, images were converted to 8-bit, and a threshold to a control pixel of each image was set. As a measure of K-Ras mislocalization, the fraction of mCherry-CAAX co-localizing with mGFP-K-RasG12V was calculated using a Manders coefficient plugin downloaded from Wright Cell Image Facility. The fraction of mCherry-CAAX co-localizing with mGFP-RasG12V (Mander's coefficient) is proportional to K-Ras mislocalization.

Proliferation Assay: Cells were seeded in 96-well plates. After 24 h, fresh growth medium supplemented with 0.1% vehicle (DMSO), or various concentrations of drugs was added and cells were grown for 72 h. Cell numbers were quantified using the CyQUANT® Cell Proliferation Assay Kit (Life Technologies), according to the manufacturer's protocol. Fluorescence measurement was used as a measure of live cell number.

### Synthetic Schemes, Reagents and Yields

Schemes 1-15 outline the general synthetic routes utilized to prepare the fendiline analogs displayed in Tables 1-2. Scheme 1 outlines a synthetic route that proceeds via an intermediate amide molecule. The required precursor amines and carboxylic acids were obtained from commercial sources. The precursor amine for the preparation of compound 15 was synthesized as shown in Scheme 2. Alternatively to Scheme 1, the desired target fendiline analogs were also synthesized by a reductive amination route as illustrated in Scheme 2 for the preparation of analog 16.

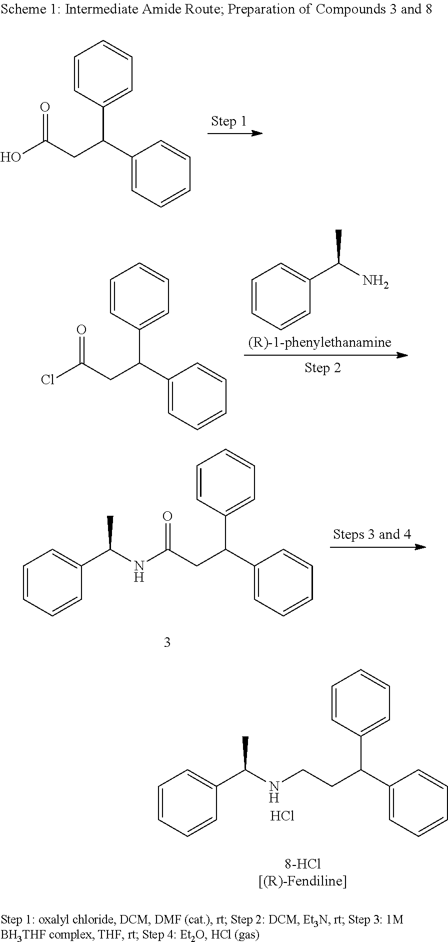

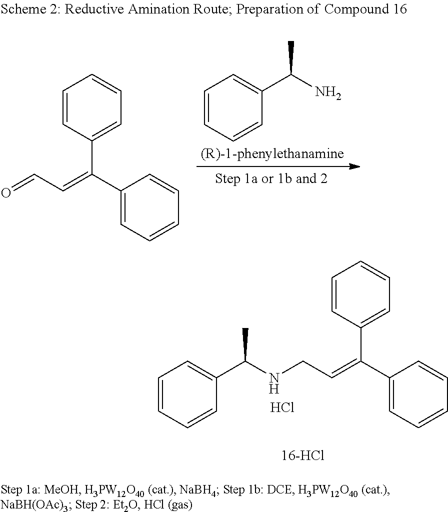

Analogs in which the basic nitrogen is replaced with an oxygen atom were prepared by the Williamson ether synthesis. This reaction is conducted by nucleophilic displacement of the appropriate alkyl bromide with the desired alkoxide (Scheme 3). Alternatively, the required alkyl tosylate (Scheme 4) or alkyl mesylate (Scheme 5) group can substitute for the bromide.

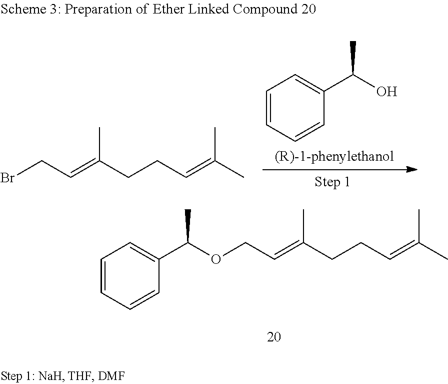

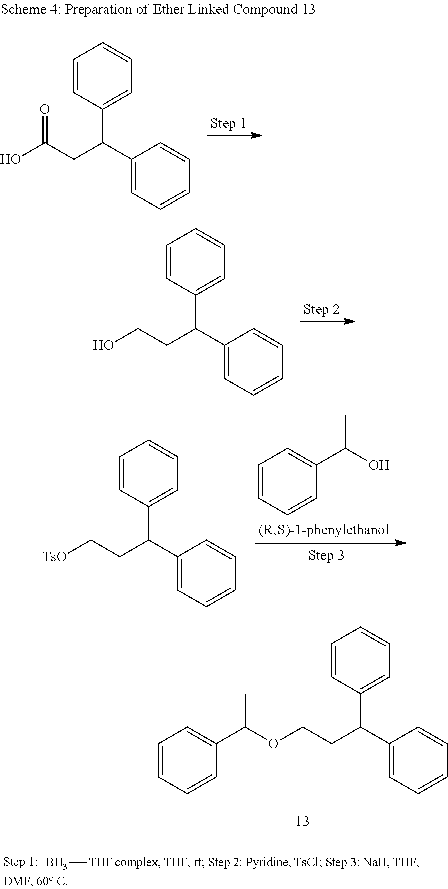

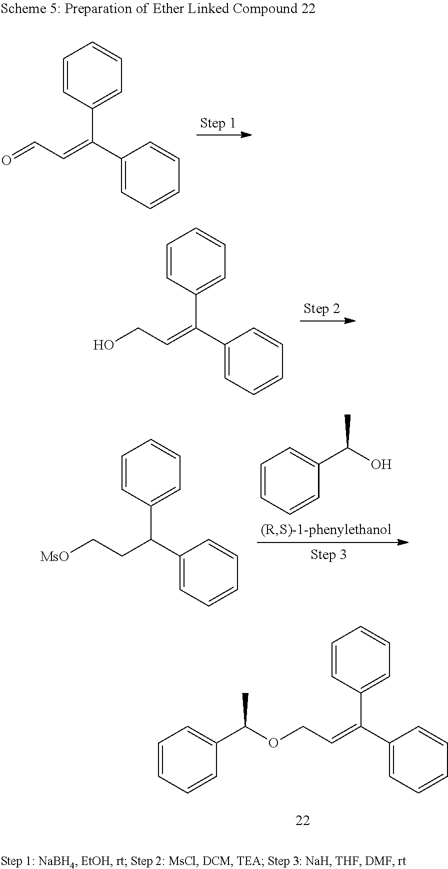

Depending upon the commercial availability of the required starting materials, precursor aldehyde compounds were obtained either by Dess-Martin periodinane oxidation of the corresponding alcohol (Scheme 6) or through Wittig reaction with the appropriate carbonyl compound (Scheme 7). The various nitro and amino substituted analogs were obtained by the synthetic sequences of Scheme 9-11.

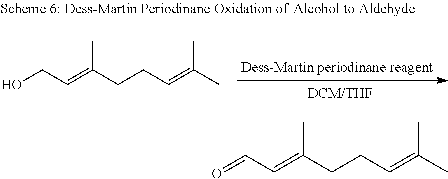

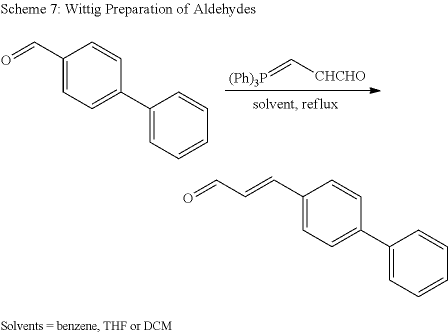

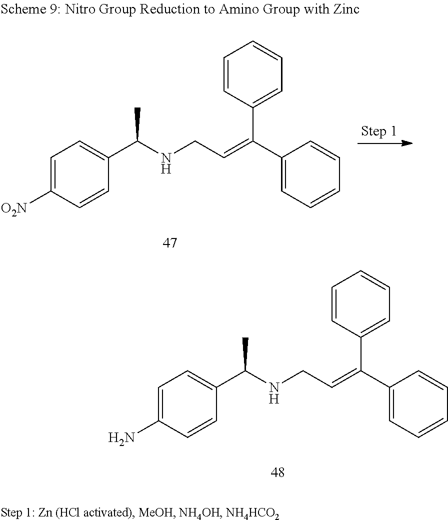 [SMILES: C[C@@H](NCC=C(c1ccccc1)c1ccccc1)c1ccc([N+](=O)[O-])cc1 ; C[C@@H](NCC=C(c1ccccc1)c1ccccc1)c1ccc(N)cc1]

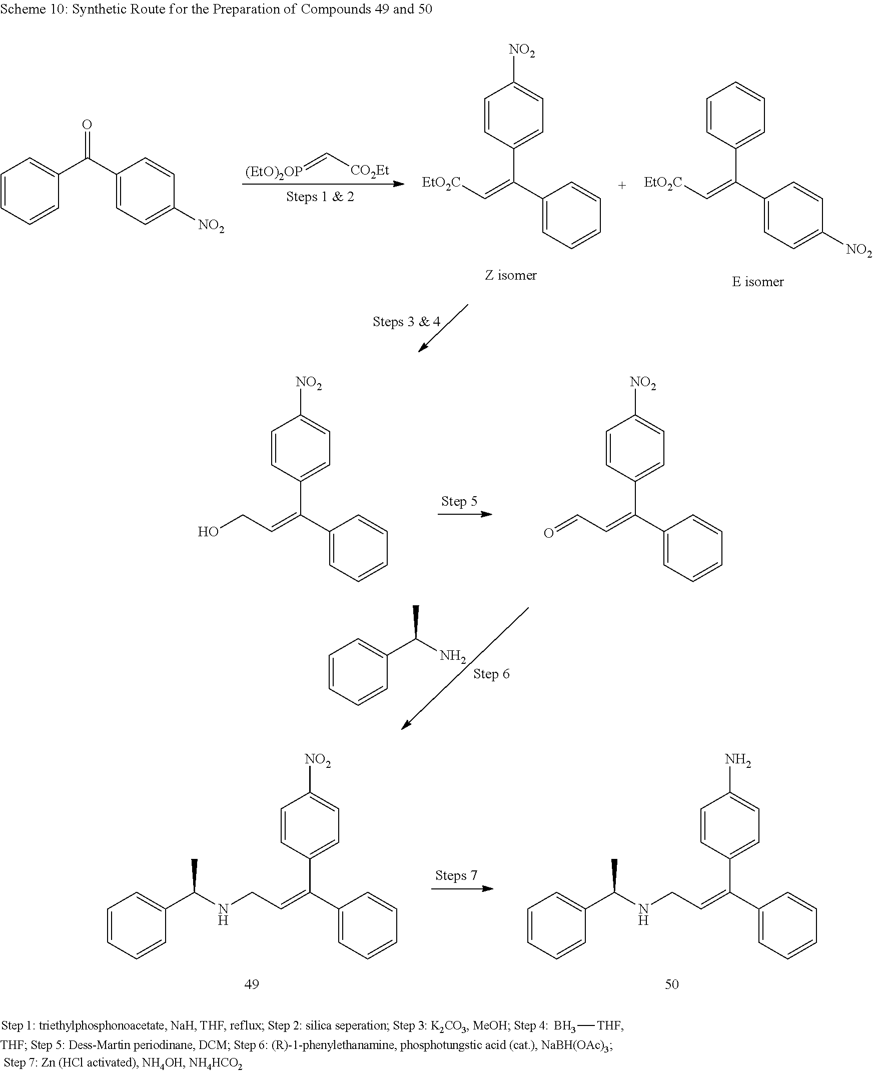

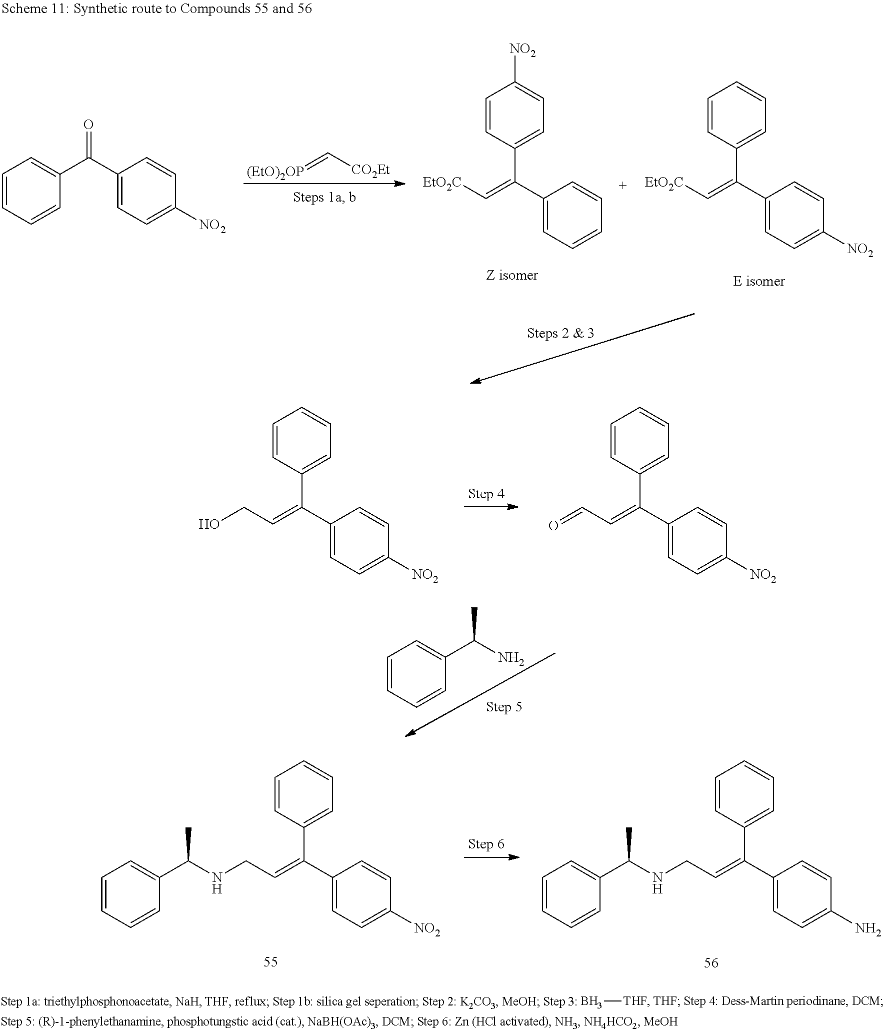

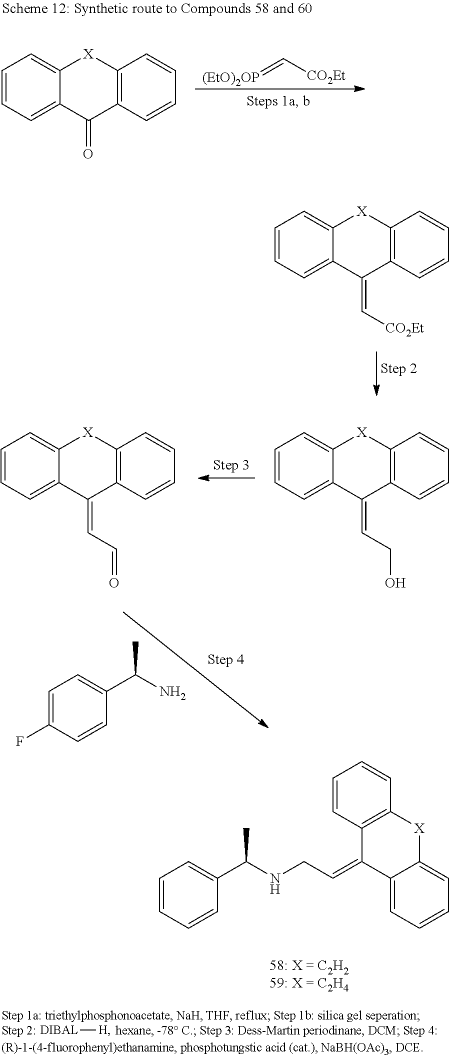

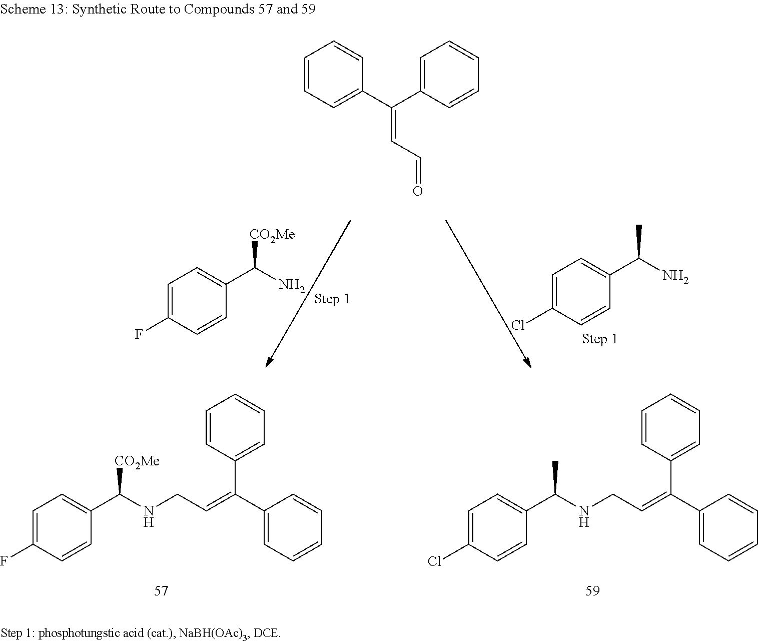

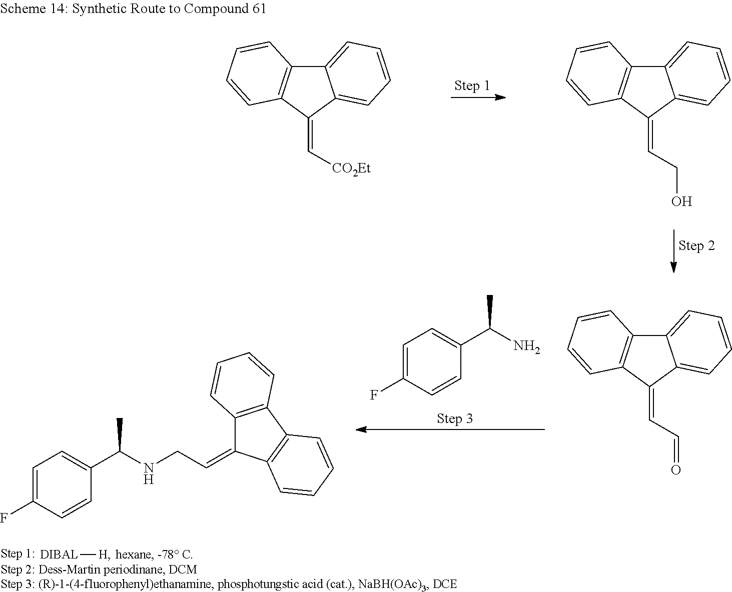

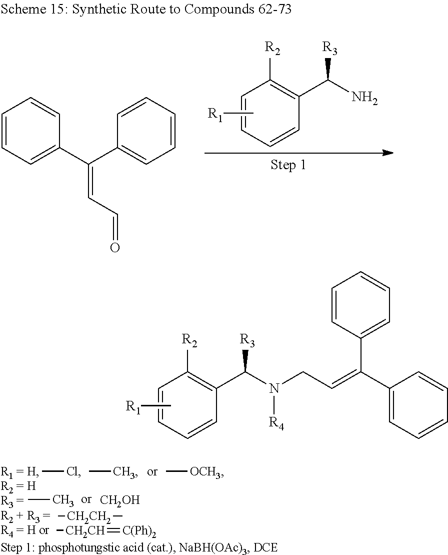

### Synthesis and Characterization of Compounds and Intermediates

### Example 1

### N-(2,2-diphenylpropyl)-2-phenylpropanamide (Compound 02)

To a RB flask under a nitrogen atmosphere was added 1.5 g (10 mmol) of 2-phenyl propanoic acid, 10 mL of DCM and 6 drops of DMF. Oxalyl chloride [2.2 g, 17.3 mmol] in 10 mL of DCM was added in small portions and the reaction was stirred at room temperature overnight. Rotary evaporation under reduced pressure yielded 2-phenylpropanoyl chloride as a yellow oil. This material was dissolved in 5 mL of DCM and treated, with cooling, with a dropwise addition of a solution composed of 2.04 g (10.35 mmol) of 2,2-diphenylethanamine, 1.5 mL (10.78 mmol) TEA in 15 mL DCM. The light yellow reaction mixture was then stirred at room temperature for 2 hours after which it was diluted with EtOAc, washed with dilute HCl, then with dilute NaHCO₃ solution, and dried over MgSO₄. Rotary evaporation under reduced pressure gave 3.69 g of yellow oil. The yellow oil was further purified by recrystallization in 30% EtOAc/hexanes and flash column chromatography on silica gel (50% DCM/hexanes to 20% EtOAc/DCM) to afford 2.99 g (91%) of Compound 02.

¹H NMR (CDCl₃, 500 MHz): δ 7.05-7.3 (m, 15H), 5.25 (t, 1N—H), 4.07 (t, J=6.4 Hz, 1H), 3.85-3.91 (m, 1H), 3.45-3.76 (m, 1H), 3.43 (q, J=5.76, 1H), 1.44 (d, J=5.8 Hz, 1H)

ESI(MS) of [C₂₃H₂₃NO+23] calculated is 352.18, found 352.2

### Example 2

### (R)-3,3-diphenyl-N-(1-phenylethyl)propanamide (Compound 03)

To a RB flask under a nitrogen atmosphere was added 4.52 g (20 mmol) of 3.3-diphenyl propionic acid, 25 mL of DCM and 10 drops of DMF. Oxalyl chloride [5.54 g, 43.6 mmol] in 15 mL of DCM was added in small portions and the reaction was stirred at rt overnight. Rotary evaporation under reduced pressure yielded 5.36 g of unpurified 3,3-diphenylpropanoyl chloride.

A solution of 2.68 g (10 mmol) of the unpurified 3,3-diphenylpropanoyl chloride in 13 mL of DCM was added dropwise to a cooled solution of 1.33 g (11 mmol) of (R)-1-phenylethanamine, 2.52 g (25 mmol) TEA and 30 mL DCM. After stirring overnight at rt, the reaction was quenched with excess 2N HCl. The organic layer was washed with water followed by dilute NaHCO₃ solution, then brine, and finally dried over MgSO₄. Rotary evaporation under reduced pressure yielded 4.06 g of yellow oil. The yellow oil was purified by recrystallization in 30% EtOAc/hexanes and flash column chromatography on silica gel (100% DCM to 5% EtOAc/DCM) to afford 3.16 g (96%) of Compound 03.

¹H NMR (CDCl₃, 500 MHz): δ 6.95-7.31 (m, 15H), 5.38 (d, J=6 Hz, 1N—H), 4.96-5.01 (m, 1H), 4.55 (t, J=6.32 Hz, 1H), 2.90 (d, J=6.32 Hz, 2H), 1.26 (d, J=5.52 Hz, 3H)

ESI(MS) of [C₂₃H₂₃NO+H] calculated is 330.18, found 330.2

### Example 3

### (S)-3,3-diphenyl-N-(1-phenylethyl)propanamide (Compound 04)

The title compound was made following the general procedure of Example 2 using 2.68 g (10 mmol) of the 3,3-diphenylpropanoyl chloride and 1.33 g (11 mmol) of (S)-1-phenylethanamine to give 3.01 g (91%) of Compound 04.

¹H NMR (CDCl₃, 500 MHz): δ 6.95-7.31 (m, 15H), 5.39 (d, J=6 Hz, 1N—H), 4.96-5.01 (m, 1H), 4.55 (t, J=6.36 Hz, 1H), 2.90 (d, J=6.32 Hz, 2H), 1.26 (d, J=5.48 Hz, 3H)

ESI(MS) of [C₂₃H₂₃NO+23] calculated is 352.18, found 352.2

### Example 4

### N-benzyl-3,3-diphenylpropanamide (Compound 05)

3,3-Diphenylpropanoyl chloride (3.01 g unpurified acid chloride obtained as in Example 2) in 15 mL DCM was added dropwise to a cooled solution of 1.17 g (11 mmol) of benzyl amine, 2.52 g (25 mmol) TEA in 30 mL DCM. After stirring at rt for 3 hours, the reaction was quenched by addition of excess 2N HCl. The layers were separated and the organic layer was washed with water, followed by dilute NaHCO₃ solution, and then dried over MgSO₄. Rotary evaporation under reduced pressure yielded 3.26 g of yellow solid. This material was purified by recrystallization in 30% EtOAc/hexanes and flash column chromatography on silica gel (100% DCM to 5% EtOAc/DCM) to afford 3.15 g (93%) of Compound 05.

¹H NMR (CDCl₃, 500 MHz): δ 7.25-7.31 (m, 15H), 5.51 (t, 1N—H), 4.62 (t, J=6.32 Hz, 1H), 4.31 (d, J=4.6 Hz, 2H), 2.94 (d, J=6.36 Hz, 2H)

ESI(MS) of [C₂₂H₂₁NO+23] calculated is 338.16, found 338.2

### Example 5

### 3-phenyl-N-(1-phenylethyl)propanamide (Compound 06)

To a RB flask under a nitrogen atmosphere was added 1.5 g (10 mmol) of 3-phenylpropanoic acid, 20 mL of DCM and 5 drops of DMF. Oxalyl chloride [2.89 g, 22.7 mmol] in 10 mL of DCM was added in small portions and the reaction was stirred at rt for 3 hours. Rotary evaporation under reduced pressure gave 3-phenylpropanoyl chloride. The acid chloride was not further purified and was dissolved in 10 mL of DCM. This solution was added dropwise with cooling to a solution composed of 1.33 g (11 mmol) of 1-phenylethanamine, 2.52 g (25 mmol) TEA in 30 mL DCM. The resulting reaction mixture was stirred at rt for 2 hours after which it was diluted with 25 mL of 2N HCl and water. The organic layer was isolated and washed with dilute NaHCO₃ solution, then water and finally dried over MgSO₄. Rotary evaporation under reduced pressure gave 3.21 g of product. The product was purified by recrystallization in 30% EtOAc/hexanes and flash column chromatography on silica gel (50% EtOAc/DCM) to afford 2.51 g (99%) of Compound 06.

¹H NMR (CDCl₃, 500 MHz): δ 7.2-7.35 (m, 10H), 5.51 (d, 1N—H), 5.07-5.13 (quintet, J=5.8 Hz,1H), 2.97 (t, J=6 Hz, 2H), 2.43-2.52 (dt, 2H), 1.41 (d, J=5.52 Hz, 3H)

### Example 6

### N-(2,2-diphenylethyl)-2-phenylpropan-1 amine hydrochloride (Compound 07)

Compound 02 (238 mg, 0.72 mmol) was dissolved in 5 mL THF and 2 mL of 1M BH₃-THF was added under a nitrogen atmosphere. After stirring overnight at rt, 3 mL of 2N HCl was added dropwise and the reaction was heated to a gentle reflux for 1 hour, then cooled and made basic (pH>10) with excess NaOH solution. This mixture was extracted with EtOAc, the organic extract was isolated and dried over MgSO₄. Rotary evaporation under reduced pressure gave 197 mg (86%) of Compound 07.

ESI(MS) of [C₂₃H₂₅N+H] calculated is 316.20, found 316.20

Approximately 128 mg (0.40 mmol) of Compound 07 was dissolved in 6 mL Et₂O and HCl (g) was bubbled through the solution. A white precipitate formed immediately which was filtered and air dried to give 66 mg of Compound 07 hydrochloride.

¹H NMR (CDCl₃, 400 MHz): δ 7.22-7.24 (m, 9H), 7.05-7.10 (m, 6H), 4.60 (t, J=8 Hz, 1H), 3.65-3.70 (m, 1H), 3.28-3.37 (m, 3H), 2.92-2.95 (m, 1H), 1.30 (d, J=6.8 Hz, 3H)

### Example 7

### (R)-3,3-diphenyl-N-(1-phenylethyl)propan-1-amine hydrochloride; [(R)-Fendiline hydrochloride] (Compound 08)

To a RB flask under a nitrogen atmosphere was added Compound 03 (0.989 g, 3 mmol) and 25 mL THF. To this was added 7 mL of 1M BH₃-THF. After stirring overnight at rt, the reaction was quenched by the dropwise addition of 3 mL of 6N HCl after which the reaction mixture was heated to a gentle reflux for 1 hour. The mixture was then cooled and condensed to ˜1/2 the volume by rotary evaporation. The residual solution was made basic (pH>10) with excess NaOH solution and then partitioned with a mixture of EtOAc and DCM. The organic layer was dried over MgSO₄ and evaporated under reduced pressure to give 3.21 g of product as a solid. The solid was purified by flash column chromatography on silica gel (EtOAc/DCM) to afford 0.91 g (96%) of Compound 08.

Approximately 456 mg (1.44 mmol) of Compound 08 was dissolved in 20 mL Et₂O and HCl (g) was bubbled through the solution. A white precipitate formed immediately which was filtered and air dried to give 292 mg of Compound 08 hydrochloride.

¹H NMR (CDCl₃, 400 MHz): δ 7.48-7.51 (m, 2H), 7.34-7.37 (m, 3H), 7.07-7.19 (m, 10H), 4.05-4.10 (m, 1H), 3.89-3.92 (m, 1H), 2.62-2.68 (m, 4H), 1.69 (d, J=6.8 Hz, 3H)

ESI(MS) of [C₂₃H₂₅N+H] calculated is 316.20, found 316.20

### Example 8

### (S)-3,3-diphenyl-N-(1-phenylethyl)propan-1-amine hydrochloride; [(S)-Fendiline hydrochloride] (Compound 09)

Compound 04 (988 mg, 3 mmol) was dissolved in 25 mL THF and 7 mL of 1M BH₃-THF was added under a nitrogen atmosphere. After stirring overnight at rt, 3 mL of 6N HCl was added dropwise and the reaction was heated to a gentle reflux for 1 hour, cooled and condensed by rotary evaporation to approximately half the volume. The residual solution was made basic (pH>10) with excess NaOH solution and then partitioned with a mixture of EtOAc and DCM. The organic layer was dried over MgSO₄. Rotary evaporation under reduced pressure gave 1.1 g of product which was further purified by flash column chromatography on silica gel (EtOAc/DCM) to afford 611 mg (65%) of Compound 09.

ESI(MS) of [C₂₃H₂₅N+H] calculated is 316.20, found 316.20

Approximately 215 mg of Compound 09 was dissolved in 20 mL Et₂O and HCl (g) was bubbled through the solution. A white precipitate formed immediately which was filtered and air dried to give 200 mg of Compound 09 hydrochloride.

¹H NMR (CDCl₃, 400 MHz): δ 7.48-7.52 (m, 2H), 7.33-7.36 (m, 3H), 7.06-7.21 (m, 10H), 4.07 (q, J=6.4 Hz, 1H), 3.89-3.93 (m, 1H), 2.62-2.66 (m, 4H), 1.69 (d, J=6.4 Hz, 3H)

### Example 9

### N-benzyl-3,3-diphenylpropan-1-amine (Compound 10)

To a RB flask under a nitrogen atmosphere was added Compound 05 (0.981 g, 3.1 mmol) in 25 mL THF followed by 7 mL of 1M BH₃-THF. After stirring the reaction overnight and rt, 3 mL of 6N HCl was added dropwise and the reaction was heated to a gentle reflux for 1 hour, cooled, made basic (pH>10) with excess NaOH solution, and extracted with EtOAc. The organic extract was washed with brine, dried over MgSO₄ and condensed by rotary evaporation to give 1.28 g of turbid colorless oil. This material was purified by flash column chromatography on silica gel (0%-100% EtOAc/hexanes) to afford 126 mg of Compound 10.

¹H NMR (CDCl₃, 500 MHz): δ 7.14-7.18 (m, 15H), 4.03 (t,J=6.28 Hz, 1H), 3.72 (s,2H), 2.60 (t, J=6 Hz, 2H), 2.43-2.52 (m, 2H)

ESI(MS) of [C₂₂H₂₃N+H] calculated is 302.18, found 302.19

### Example 10

### 3-phenyl-N-(1-phenylethyl)propan-1-amine hydrochloride (Compound 11)

Compound 06 (1.26 g, 5 mmol) was dissolved in 30 mL THF and 10 mL of 1M BH₃-THF was added under a nitrogen atmosphere. After overnight at rt, 3 mL of 6N HCl was added dropwise and the reaction was heated to a gentle reflux for 1 hour, cooled, made basic (pH>10) with excess NaOH solution and extracted with EtOAc. The EtOAc extract was washed with brine, dried over MgSO₄ and condensed by rotary evaporation to yield 1.31 g of unpurified Compound 11.

ESI(MS) of [C₁₇H₂₁N+H] calculated is 239.17, found 240.17

Unpurified Compound 11 [0.51 g, (2.13 mmol)] was dissolved in 20 mL of Et₂O and HCl(g) was bubbled into the solution. A white milky solution formed which disappeared after a few minutes. The oil which resulted after rotary evaporation was dissolved in DCM.

Evaporation of the DCM yielded 162 mg of a tacky glass which upon standing at rt formed crystalline Compound 11 hydrochloride.

### Example 11

### 3,3-diphenyl-N-(1-phenylethyl)prop-2-en-1-amine hydrochloride (Compound 12)

To a round bottom flask containing 0.266 g (2.2 mmol) of 1-phenylethanamine was added 5 mL of MeOH, 0.416 g (2 mmol) of 3-phenyl cinnamaldehyde and 30 mg of phosphotungstic acid hydrate. The reaction mixture was stirred vigorously for 10 minutes before adding 25 mg of NaBH₄. The reaction mixture turned deep blue purple with evolution of gas which faded to dirty grey in about 30 minutes. The reaction mixture was diluted with 1:1 EtOAc/hexanes (25 mL), and extracted with saturated NaHCO₃, water, brine, dried over MgSO₄ and condensed by rotary evaporation. The resulting crude product was purified by flash column chromatography on silica gel (0-10% EtOAc/hexanes) to afford 543 mg (86%) of Compound 12.

¹H NMR (CDCl₃, 500 MHz): δ 7.08-7.33 (m, 15H), 6.17 (t, J=5.6 Hz, 1H), 3.75 (q, J=5.2 Hz, 1H), 3.18 (d, J=5.6 Hz, 2H), 1.31 (d, J=5.3 Hz, 3H)

ESI(MS) of [C₂₃H₂₃N+H] calculated is 313.2, found 314.2

Approximately 400 mg (1.27 mmol) of Compound 12 was dissolved in about 15 mL Et₂O and HCl (g) was bubbled through the solution. A white precipitate formed immediately which was filtered and air dried to give 415 mg of Compound 12 hydrochloride.

### Example 12

### 3-(1-phenylethoxy)propane-1,1-diyl)-dibenzene (Compound 13)

The synthesis of the title compound was achieved in two steps:

### Step 1: 3,3-diphenylpropyl 4-methylbenzenesulfonate

2.26 g (10 mmol) of 3,3-diphenylpropanoic acid was dissolved in EtOAc and 14 mL (14 mmol) of 1M BH₃-THF was added dropwise. After stirring at rt for 3 hours, the reaction was diluted with saturated NaHCO₃ solution, the layers were separated and the organic layer was dried (MgSO₄), filtered and concentrated by rotary evaporation to yield 2.19 g colorless oil which was not further purified. The oil was mixed with 25 mL pyridine and 1.9 g (10 mmol) of TsCl was added in small portions after which the reaction mixture was stirred at rt overnight. The reaction was worked up diluting with DCM and excess 3N HCl, the layers were separated and the organic layer was dried (MgSO₄), filtered and concentrated by rotary evaporation to yield 2.38 g (65%) of 3,3-diphenylpropyl 4-methylbenzenesulfonate as a colorless oil.

### Step 2: 3-(1-phenylethoxy)propane-1,1-diyl)-dibenzene

To a RB flask under a nitrogen atmosphere and containing a stirred suspension of 60% NaH (452 mg) in 10 mL THF was added dropwise 1.22 g (10 mmol) of 1-phenylethanol. The reaction was stirred at room temperature for 10 minutes before adding the freshly prepared solution of 3,3-diphenylpropyl-4-methylbenzenesulfonate (238 mg, 6.49 mmol) in 10 mL DMF under N₂ atmosphere. After heating at 60° C. for 2 hours, the reaction was diluted with water and extracted with 20% DCM/hexanes. The organic layer was washed several times with water, then brine, dried over MgSO₄ and condensed by rotary evaporation under reduced pressure. The resulting oil was purified by flash column chromatography on silica gel (0-40% DCM/hexanes) to give 285 mg (14%) of Compound 13.

¹H NMR (CDCl₃, 500 MHz): δ 7.09-7.3 (m, 15H), 4.28 (q, J=5.2 Hz, 1H), 4.10 (t, J=6.28 Hz, 1H), 3.18-3.29 (m, 2H), 2.24-2.36 (m, 2H), 1.41 (d, J=5.16 Hz, 3H)

### Example 13

### 3,3-diphenyl-N-(1-(thiophen-2-yl)ethyl)propanamide (Compound 14)

The synthesis of the title compound was achieved in 3 steps.

### Step 1: 1-(thiophen-2-yl)ethanone oxime

A RB flask was charged with 5.04 g (40 mmol) of 2-acetylthiophene, 100 mL of absolute EtOH, 7 mL pyridine and 5.56 g (40 mmol) of hydroxylamine hydrochloride. The reaction was refluxed for 2 hours and then allowed to stir at rt overnight. The reaction was then concentrated by rotary evaporation at reduced pressure and then diluted with 600 mL of ice-cold water to give a white solid which was filtered and air dried to yield 3.78 g (67%) of a syn/anti mixture of 1-(thiophen-2-yl)ethanone oxime.

### Step 2: 1-(thiophen-2-yl)ethanamine

A 100 mL RB flask was charged with 40 mL THF, flushed with nitrogen, and 1.04 g (27.4 mmol) of LAH was carefully added to the stirred THF. After a short period, 2.82 g (20 mmol) of 1-(thiophen-2-yl)ethanone oxime was added in small portions and the reaction was gently refluxed for 4-5 hours. The reaction was worked up by dropwise addition of brine and EtOAc. After all of the excess LAH was destroyed, the reaction mixture was filtered through celite. The aqueous layer was extracted with DCM and the combined organic layers were dried over MgSO₄, filtered and concentrated by rotary evaporation under reduced pressure yielded a dark-colored oil. This material was separated by flash column chromatography on silica gel (0-50% EtOAc/hexanes) to yield 0.98 g of 1-(thiophen-2-yl)ethanamine and which was used directly in step 3.

### Step 3: 3,3-diphenyl-N-(1-(thiophen-2-yl)ethyl)propanamide (Compound 14)

To a RB flask under a nitrogen atmosphere was added 1.36 g (6 mmol) of 3.3-diphenyl propionic acid, 10 mL of DCM and 2 drops of DMF. A solution of 1.63 g of oxalyl chloride in 5 mL of DCM was added in small portions and the reaction was stirred at room temperature overnight. Isolation by rotary evaporation under reduced pressure yielded 3,3-diphenylpropanoyl chloride. The 3,3-diphenylpropanoyl chloride in 10 mL of DCM was added drop wise to a solution of 0.98 g of 1-(thiophen-2-yl)ethanamine, 2.0 g of TEA and 20 mL DCM in ice-cold conditions. The reaction was then allowed to stir at rt for an hour after which it was washed with excess 2N HCl and water. The layers were separated and the organic layer was washed with dilute NaHCO₃ solution, brine, dried over MgSO₄ and evaporated under reduced pressure to give 2.44 g of oil which solidified upon standing. This material was purified by flash column chromatography on silica gel (0 to 5% EtOAc/DCM) to afford 0.140 g of Compound 14.

¹H NMR (CDCl₃, 400 MHz): δ 7.14-7.30 (m, 11H), 6.86-6.88 (m, 1H), 6.66-6.73 (m, 1H), 5.26 (quintet, J=6.8 Hz, 1H), 4.56 (t, J=8 Hz, 1H), 2.84-2.94 (m, 2H), 1.32 (d, J=6.8 Hz, 3H)

ESI(MS) of [C₂₁H₂₁NOS+Na] calculated is 358.13, found 358.1

### Example 14

### 3,3-diphenyl-N-(1-(thiophen-2-yl)ethyl)propan-1-amine hydrochloride (Compound 15)

Compound 14 (0.698 g, 2.07 mmol) was dissolved in 30 mL THF and 8 mL of 1M BH₃-THF was added under a nitrogen atmosphere. After stirring at rt overnight, 4 mL of 6N HCl was added dropwise and the reaction was heated to a gentle reflux for 1 hour, cooled, made basic (pH>10) with excess NaOH solution and extracted with EtOAc. The crude materials were dried over MgSO₄ and concentrated by rotary evaporation under reduced pressure to yield 1.12 g of dark orange-yellow oil. This material was purified by flash column chromatography on silica gel (0-50% EtOAc/hexanes) to afford 0.962 g of Compound 15.

Approximately 746 mg of Compound 15 was dissolved in 15 mL Et₂O and HCl (g) was bubbled through the solution to give an insoluble oil. The Et₂O was decanted and upon standing overnight the oil converted into an off-white solid. This material was purified by trituration with hot 10% EtOAc/hexanes to give Compound 15 hydrochloride.

¹H NMR (CDCl₃, 400 MHz): δ 7.09-7.34 (m, 12H), 6.97-6.99 (m, 1H), 4.39 (m, 1H), 3.99 (t, J=6.8 Hz, 1H), 2.64-2.73 (m, 4H), 1.76 (d, J=6.8 Hz, 3H)

ESI(MS) of [C₂₁H₂₃NS+H] calculated is 322.16, found 322.2

### Example 15

### (R)-3,3-diphenyl-N-(1-phenylethyl)prop-2-en-1-amine (Compound 16)

To a RB flask containing 0.144 g (1.19 mmol) of (R)-1-phenylethanamine was added 5 mL of MeOH, 0.225 g (1.08 mmol) of 3-phenyl cinnamaldehyde and 20 mg of phosphotungstic acid hydrate. The reaction mixture was stirred vigorously for 10 minutes before adding 40 mg (1.08 mmol) of NaBH₄. The reaction mixture turned deep blue purple with evolution of gas. The blue color faded to dirty grey in about 30 minutes after the mixture was diluted with 1:1 EtOAc/hexanes (25 mL), extracted with saturated NaHCO₃, water and brine, and then dried over MgSO₄. Rotary evaporation under reduced pressure yielded 0.26 g of material that was purified by flash column chromatography on silica gel (0-10% EtOAc/hexanes) to afford 73 mg (31.4%) of Compound 16.

¹H NMR (CDCl₃, 400 MHz): δ 7.08-7.33 (m, 15H), 6.16 (t, J=6.8 Hz, 1H), 3.74 (q, J=6.4 Hz, 1H), 3.18 (d, J=6.8 Hz, 2H), 1.31 (d, J=6.4 Hz, 3H)

ESI(MS) of [C₂₃H₂₃N+H] calculated is 314.18, found 314.2

### Example 16

### (S)-3,3-diphenyl-N-(1-phenylethyl)prop-2-en-1-amine (Compound 17)

Compound 17 was obtained following the general procedure of Example 15, using 144 mg (1.19 mmol) of (S)-1-phenylethanamine and 0.214 g (1.03 mmol) of 3-phenyl cinnamaldehyde to yield 80 mg (25%) of Compound 17.

¹H NMR (CDCl₃, 400 MHz): δ7.08-7.29 (m, 15H), 6.17 (t, J=6.8 Hz, 1H), 3.74 (q, J=6.4 Hz, 1H), 3.18 (d, J=6.8 Hz, 2H), 1.31 (d, J=6.4 Hz, 3H)

ESI(MS) of [C₂₃H₂₃N+H] calculated is 314.18, found 314.2

### Example 17

### (R,E)-3-phenyl-N-(1-phenylethyl)prop-2-en-1-amine (Compound 18)

To a RB flask containing 0.144 g (1.19 mmol) of (R)-1-phenylethanamine was added 5 mL of MeOH, 0.142 g (1.08 mmol) of trans-cinnamaldehyde and 15 mg of phosphotungstic acid hydrate. The reaction mixture was stirred vigorously for 10 minutes before adding 40 mg (1.08 mmol) of NaBH₄. The reaction mixture turned dark green with evolution of gas and the color faded to dirty grey in about 60 minutes. The mixture was diluted with 1:1 EtOAc/hexane (25 mL), extracted with saturated NaHCO₃, water and brine, and dried over MgSO₄. Rotary evaporation under reduced pressure yielded crude product which was purified by flash column chromatography on silica gel (0-100% EtOAc/hexanes) to afford 59 mg (23%) of Compound 18.

¹H NMR (CDCl₃, 400 MHz): δ 7.18-7.35 (m, 10H), 6.45 (d, J=16 Hz, 1H), 6.23-6.31 (m, 1 Hz), 4.11 (q, J=7.2 Hz, 1H), 3.27 (d, J=6.4 Hz, 2H), 1.38 (d, J=6.8 Hz, 3H)

ESI(MS) of [C₁₇H₁₉N+H] calculated is 238.15, found 238.1

### Example 18

### (S,E)-3-phenyl-N-(1-phenylethyl)prop-2-en-1-amine (Compound 19)

Compound 19 was obtained following the general procedure of Example 17, using 146 mg (1.20 mmol) of (S)-1-phenylethanamine and 0.138 mg (1.04 mmol) of trans-cinnamaldehyde to yield 31 mg (12%) of Compound 19.

¹H NMR (CDCl₃, 400 MHz): δ 7.18-7.35 (m, 10H), 6.46 (d, J=24 Hz, 1H), 6.24-6.31 (m, 1 Hz), 3.85 (q, J=6.4 Hz, 1H), 3.26 (d, J=6.0 Hz, 2H), 1.39 (d, J=6.4 Hz, 3H)

ESI(MS) of [C₁₇H₁₉N+H] calculated is 238.15, found 238.1

(R)-1-phenylethanol (611 mg, 5 mmol) was added dropwise to a RB flask containing a suspension of 60% NaH (400 mg, 10 mmol) in 10 mL of THF under a nitrogen atmosphere. After stirring at rt for 15 minutes, geranyl bromide (1.086 g, 5 mmol) in 10 mL DMF was added dropwise and the flask was flushed with nitrogen and stirred at rt overnight. The resulting deep-orange reaction mixture was diluted with water and extracted with hexanes. The hexanes layer was washed with brine, dried over MgSO₄ and condensed by rotary evaporation under reduced pressure to a crude oil which was purified by flash column chromatography on silica gel (0-35% DCM/hexanes) to afford 283 mg of Compound 20, mixture of cis and trans forms in proportion to the composition of the starting geranyl bromide.

¹H NMR (CDCl₃, 400 MHz): δ 7.25-7.35 (m, 5H), 5.35 (m, 1H), 5.08 (m, 1H), 4.43 (q, J=6.4 Hz, 1H), 3.80-3.86 (m, 2H), 1.97-2.10 (m, 4H), 1,67 (s, 3H), 1.59 (s, 3H), 1.54 (s, 3H), 1.44 (d, J=6.4 Hz, 3H)

ESI(MS) of [C₁₈H₂₇N+H] calculated is 258.21, found 258.2

### Example 20

### (R,E)-3,7-dimethyl-N-(1-phenylethyl)octa-2,6-dien-1-amine (Compound 21)

The title compound was prepared in two steps.

### Step 1: (E)-3,7-dimethylocta-2,6-dienal (geranial)

To a RB containing 331 mg (2.15 mmol) geraniol in 1:1 DCM/THF was added 915 mg (2.15 mmol) of Dess-Martin periodinane in small portions. The reaction mixture turned turbid with the formation of a white precipitate. After 40 minutes of stirring at rt the reaction was diluted with DCM and washed with water and brine. The organic layer was dried over MgSO₄, filtered and condensed by rotary evaporation to give a white oily solid which was further purified by flash column chromatography on silica gel (0-20% ether/hexanes) to yield 246 mg of (E)-3,7-dimethylocta-2,6-dienal (geranial).

### Step 2: (R,E)-3,7-dimethyl-N-(1-phenylethyl)octa-2,6-dien-1-amine

To a RB flask containing 0.232 g (1.92 mmol) of (R)-1-phenylethanamine was added 5 mL of MeOH, 0.246 g (1.62 mmol) of geranial and 22 mg of phosphotungstic acid hydrate. The reaction mixture was stirred vigorously for 10 minutes before adding 24 mg of NaBH₄. The reaction mixture turned dark green with evolution of gas and the color faded to dirty grey in about 30 minutes. The mixture was diluted with DCM and extracted with saturated NaHCO₃, water and brine, and then dried over MgSO₄. Rotary evaporation under reduced pressure yielded crude product which was further purified by flash column chromatography on silica gel (0-2% methanol/DCM) to afford 197 mg of Compound 21.

¹H NMR (CDCl₃, 400 MHz): δ 7.21-7.34 (m, 5H), 5.23-5.30 (m, 1H), 5.01-5.10 (m, 1H), 3.77 (m, 1H), 3.02-3.10 (m, 2H), 1.94-2.10 (m, 4H), 1.68-1.70 (s, 3H), 1.59-1.64 (s, 3H), 1.52-1.55 (s, 3H), 1.34-1.37 (m, 3H)

### Example 21

### (R)-(3-(1-phenylethoxy)prop-1-ene-1,1-diyl)dibenzene (Compound 22)

The title compound was achieved in 3 steps:

### Step 1: 3-phenylcinnamyl alcohol

To a solution of 53 mg (1.41 mmol) of NaBH₄ in 2 mL EtOH, was added a solution of 0.74 g (3.54 mmol) of 3-phenyl cinnamaldehyde in 4 mL of EtOH. After stirring the reaction mixture at rt for 15 minutes, approximately 75 mL of 1:1 water and Et₂O was added. The ethereal layer was washed with water, brine, and then dried over MgSO₄. The filtered solution was condensed by rotary evaporation under reduced pressure to give 0.62 g (83%) of 3-phenylcinnamyl alcohol.

### Step 2: 3,3-diphenylallyl methanesulfonate

To a solution of 0.284 g (1.35 mmol) of 3-phenylcinnamyl alcohol in 3 mL of anhydrous DCM, was added 0.41 mL (2.97 mmol) of TEA. The reaction mixture was cooled to 0° C. and 0.16 mL (2.02 mmol) of MsCl was added dropwise. The mixture was warmed to rt overnight. The product was isolated by diluting the reaction mixture with Et₂O and extracting with saturated NaHCO₃, washing with water and brine, drying over MgSO₄ and rotary evaporating under reduced pressure to give 225 mg (58%) of 3,3-diphenylallyl methanesulfonate.

### Step 3: (R)-(3-(1-phenylethoxy)prop-1-ene-1,1-diyl)dibenzene

(R)-1-phenylethanol [94 μL, 0.78 mmol] was added dropwise to a suspension of 60% NaH (69 mg, 1.71 mmol) in 2.5 mL THF. The reaction was stirred at room temperature for 10 minutes before adding a solution of 3,3-diphenylallyl methanesulfonate (225 mg, 0.78 mmol) in 1.5 mL DMF under a nitrogen atmosphere. After stirring at rt for 90 minutes, the reaction was diluted with water and Et₂O (1:1). The ethereal layer was washed several times with water, then brine, dried over MgSO₄ and rotary evaporated under reduced pressure. The resulting crude product was purified by flash column chromatography on silica gel (0-35% DCM/hexanes) to give 76 mg (31%) of Compound 22.

¹H NMR (CDCl₃, 400 MHz): δ 7.09-7.28 (m, 15H), 6.21 (t, J=7.2 Hz, 1H), 4.39 (q, J=6.4 Hz, 1H), 3.90-3.98 (m, 2H), 1.45 (d, J=6.4 Hz, 3H)

### Example 22

### (S)-(3-(1-phenylethoxy)prop-1-ene-1,1-diyl)dibenzene (Compound 23)

Compound 23 was obtained following the general procedure of Example 21 using 0.13 mL (1.075 mmol) of (S)-1-phenylethanol and 310 mg (1.075 mmol) of 3,3-diphenylallyl methanesulfonate to give 65 mg (19%) of Compound 23.

¹H NMR (CDCl₃, 400 MHz): δ 7.09-7.31 (m, 15H), 6.21 (t, J=7.2 Hz, 1H), 4.39 (q, J=6.4 Hz, 1H), 3.87-3.98 (m, 2H), 1.45 (d, J=6.4 Hz, 3H)

### Example 23

### (R,E)-(1-(cinnamyloxy)ethyl)benzene (Compound 24)

(R)-1-phenylethanol [0.49 mL, 4.09 mmol] was added dropwise to a suspension of 60% NaH (360 mg, 9.004 mmol) in 2.5 mL THF. The reaction was stirred at rt for 10 minutes before adding cinnamyl bromide (0.61 mL, 4.09 mmol) in 4 mL DMF under a nitrogen atmosphere. After stirring at rt for 90 minutes, the reaction was diluted with water and Et₂O (1:1). The ethereal layer was washed several times with water, then brine, dried over MgSO₄ and rotary evaporated under reduced pressure. The resulting crude product was purified by flash column chromatography on silica gel (0-35% DCM/hexanes) to give 360 mg (37%) of Compound 24.

¹H NMR (CDCl₃, 400 MHz): δ 7.20-7.38 (m, 10H), 6.55 (d, J=16 Hz, 1H), 6.25-6.32 (m, 1H), 4.50 (q, J=6.4 Hz, 1H), 3.94-4.08 (dq, 2H), 1.48 (d, J=6.4 Hz, 3H)

### Example 24

### (S,E)-(1-(cinnamyloxy)ethyl)benzene (Compound 25)

Compound 25 was made following the general procedure of Example 23 using 0.49 mL (4.09 mmol) of (S)-1-phenylethanol and 0.61 mL (4.09 mmol) of cinnamyl bromide to give 324 mg (33%) of Compound 25.

¹H NMR (CDCl₃, 400 MHz): δ 7.20-7.38 (m, 10H), 6.55 (d, J=16 Hz, 1H), 6.25-6.32 (m, 1H), 4.52 (q, J=6.4 Hz, 1H), 3.94-4.08 (dq, 2H), 1.48 (d, J=6.4 Hz, 3H)

To a RB flask containing 0.150 g (0.7497 mmol) of 1-(4-bromophenyl)ethanamine was added 5 mL of MeOH, 0.156 g (0.7497 mmol) of 3-phenyl cinnamaldehyde and 11 mg of phosphotungstic acid hydrate. The reaction mixture was stirred vigorously for 10 minutes before adding 29 mg (0.7479 mmol) of NaBH₄. The reaction mixture turned deep blue purple with evolution of gas after which the color faded to milkish yellow in about 45 minutes. The reaction mixture was diluted with 1:1 EtOAc/hexanes (25 mL) and extracted with saturated NaHCO₃, water and brine, and dried over MgSO₄. Rotary evaporation under reduced pressure yielded the crude product which was purified by flash column chromatography on silica gel (0-100% EtOAc/hexanes) to afford 72 mg (24.4%) of Compound 26.

¹H NMR (CDCl₃, 400 MHz): δ 7.06-7.39 (m, 14H), 6.13 (t, J=6.8 Hz, 1H), 3.74 (q, J=6.4 Hz, 1H), 3.14 (d, J=7.2 Hz, 2H), 1.27 (d, J=6.8 Hz, 3H)

ESI(MS) of [C₂₃H₂₂BrN+H] calculated is 392.09, found 392.1

### Example 26

### N-(1-(4-fluorophenyl)ethyl)-3,3-diphenylprop-2-en-1-amine (Compound 27)

To a RB flask containing 0.150 g (1.0778 mmol) of 1-(4-fluorophenyl)ethanamine was added 5 mL of MeOH, 0.204 g (0.9798 mmol) of 3-phenyl cinnamaldehyde and 14 mg of phosphotungstic acid hydrate. The reaction mixture was stirred vigorously for 10 minutes before adding 37 mg (0.9798 mmol) of NaBH₄. Reaction mixture turned deep blue purple with evolution of gas and the color faded to turbid white in about 60 minutes. The reaction mixture was diluted with 1:1 EtOAc/hexanes and extracted with saturated NaHCO₃, water and brine, and then dried over MgSO₄. Rotary evaporation under reduced pressure yielded the crude product which was purified by flash column chromatography on silica gel (0-50% EtOAc/hexanes) to afford 157 mg (48.4%) of Compound 27.

¹H NMR (CDCl₃, 400 MHz): δ 6.92-7.32 (m, 14H), 6.15 (t, J=6.8 Hz, 1H), 3.73 (q, J=6.4 Hz, 1H), 3.15 (d, J=7.2 Hz, 2H), 1.28 (d, J=7.2 Hz, 3H)

ESI(MS) of [C₂₃H₂₂FN+H] calculated is 332.17, found 332.2

### Example 27

### (2E,6E)-3,7,11-trimethyl-N—((R)-1-phenylethyl)dodeca-2,6,10-trien-1-amine (Compound 28)

The title compound was prepared in two steps.

### Step 1: Farnesal

To a RB containing 200 mg (0.899 mmol) farnesol in 2 mL DCM was added a solution of 434 mg (1.03 mmol) of Dess Martin periodinane in DCM. The reaction mixture turned turbid with the formation of a white precipitate. After 40 minutes of stirring at room temperature the reaction was diluted with Et₂O and saturated NaHCO₃ solution and then stirred for 10 minutes. The ethereal layer was separated and washed with saturated NaHCO₃, water and brine, and dried over MgSO₄. Rotary evaporation gave 228 mg of farnesal, which was used directly in the Step 2 without further purification.

### Step 2: (2E,6E)-3,7,11-trimethyl-N—((R)-1-phenylethyl)dodeca-2,6,10-trien-1-amine

To a RB flask containing 0.138 g (1.138 mmol) of (R)-1-phenylethanamine was added 5 mL of MeOH, 0.228 g (1.03 mmol) of farnesal and 15 mg of phosphotungstic acid hydrate. The reaction mixture was stirred vigorously for 10 minutes before adding 39 mg (1.03 mmol) of NaBH₄. Reaction mixture turned dark green with evolution of gas after which the color faded to yellow in about 30 minutes. The reaction mixture was diluted with 1:1 EtOAc/hexanes and extracted with saturated NaHCO₃, water and brine, and dried over MgSO₄. Rotary evaporation under reduced pressure gave a yellow colored oil. The oil was purified by flash column chromatography on silica gel (0-5% methanol/DCM) to afford 43 mg of Compound 28.

¹H NMR (CDCl₃, 400 MHz): δ 7.26-7.36 (m, 5H), 5.29-5.31 (m, 1H), 5.07-5.10 (m, 1H), 3.87-3.88 (m, 1H), 3.08-3.13 (m, 2H), 1.91-2.17 (m, 8H), 1.24-1.42 (m, 12H), 1.24-1.31 (m, 3H)

ESI(MS) of [C₂₃H₃₅N+H] calculated is 326.53, found 326.3

### Example 28

### 3,3-diphenyl-N-(1-phenylpropyl)prop-2-en-1-amine hydrochloride (Compound 29)

To a RB flask containing 0.157 g (1.1663 mmol) of 1-phenylpropan-1-amine was added 5 mL of MeOH, 0.220 g (1.06027 mmol) of 3-phenyl cinnamaldehyde and 15 mg of phosphotungstic acid hydrate. The reaction mixture was stirred vigorously for 10 minutes before adding 40 mg (1.06027 mmol) of NaBH₄. The reaction mixture turned deep blue purple with evolution of gas after which the color faded to milkish white in about 30 minutes. The reaction mixture was diluted with 1:1 EtOAc/hexanes, washed with saturated NaHCO₃, water and brine, and then dried over MgSO₄. Rotary evaporation under reduced pressure provided the crude product which was further purified by flash column chromatography on silica gel (0-40% EtOAc/hexanes) to afford Compound 29.

¹H NMR (CDCl₃, 400 MHz): δ 7.06-7.30 (m, 15H), 6.15 (t, J=6.8 Hz, 1H), 3.45 (q, J=6.0 Hz, 1H), 3.10-3.19 (m, 2H), 1.54-1.76 (m, 2H), 0.78 (t, J=7.2 Hz, 3H)

Compound 29 was dissolved in about 10 ml Et₂O and HC1(g) was bubbled through the solution. A white precipitate formed immediately which was filtered and air dried to give 40 mg of Compound 29 hydrochloride.

¹H NMR (CD₃OD, 400 MHz): 7.40-7.48 (m, 3H), 7.30-7.34 (m, 8H), 7.21-7.24 (m, 2H), 7.02-7.04 (m, 2H), 6.08 (t, J=7.2 Hz, 1H), 4.09 (dd, J=10.8, 4.4 Hz), 3.48-3.62 (m, 2H), 1.90-2.09 (m, 2H), 0.78 (t, J=7.6 Hz, 3H)

ESI(MS) of [C₂₄H₂₅N+H] calculated is 328.20, found 328.2

### Example 29

### 3,3-diphenyl-N-(2-phenylpropyl)prop-2-en-1-amine (Compound 30)

To a RB flask containing 0.170 g (1.1663 mmol) of 2-phenylpropan-1-amine was added 5 mL of MeOH, 0.238 g (1.147 mmol) of 3-phenyl cinnamaldehyde and 21 mg of phosphotungstic acid hydrate. The reaction mixture was stirred vigorously for 10 minutes before adding 43 mg (1.06027 mmol) of NaBH₄. The reaction mixture turned deep blue purple with evolution of gas after which the color faded to milkish white in about 30 minutes. The mixture was diluted with 1:1 EtOAc/hexanes, washed with saturated NaHCO₃, water and brine, and dried over MgSO₄. Rotary evaporation under reduced pressure yielded the crude product which was further purified by flash column chromatography on silica gel (0-100% EtOAc/hexanes) to afford Compound 30.

¹H NMR (CDCl₃, 400 MHz): δ 7.08-7.31 (m, 15H), 6.11 (t, J=6.8 Hz, 1H), 3.23-3.28 (m, 2H), 2.84-2.89 (m, 1H), 2.73-2.76 (m, 2H), 1.22 (d, J=7.2 Hz, 3H)

ESI(MS) of [C₂₄H₂₅N+H] calculated is 328.20, found 328.2

### Example 30

### (R,E)-3-([1,1′-biphenyl]-4-yl)-N-(1-phenylethyl)prop-2-en-1-amine (Compound 31)

The synthesis of the title compound was achieved in two steps:

### Step 1: (E)-3-([1,1′-biphenyl]-4-yl)acrylaldehyde

Biphenyl-4-carboxyaldehyde (0.199 g, 1.092 mmol) was dissolved in DCM. 2-(triphenylphosphoranylidene)acetaldehyde (0.664 g, 2.184 mmol) was added to the above solution under a nitrogen atmosphere and the reaction was refluxed overnight. The resultant orange-red reaction mixture was loaded directly onto a silica gel column and eluted with 100% Et₂O. Rotary evaporation of the pooled product fractions yielded the product with impurities. This material was further chromatographed on silica gel using 0-15% EtOAc/hexanes to give 102 mg of (E)-3-([1,1′-biphenyl]-4-yl)acrylaldehyde.

### Step 2: (R,E)-3-([1,1′-biphenyl]-4-yl)-N-(1-phenylethyl)prop-2-en-1-amine hydrochloride

To a RB flask containing 0.065 g (0.539 mmol) of (R)-1-phenylethanamine was added 5 mL of MeOH, 0.102 g (0.4902 mmol) of 3-([1,1′-biphenyl]-4-yl)acrylaldehyde and 7 mg of phosphotungstic acid hydrate. The reaction mixture was stirred vigorously for 10 minutes before adding 19 mg (0.4902 mmol) of NaBH₄. Reaction mixture turned deep blue purple with evolution of gas after which the color faded to light green in about 50 minutes. The reaction mixture was diluted with 1:1 EtOAc/hexanes, extracted with saturated NaHCO₃, water and brine, and dried over MgSO₄. Rotary evaporation under reduced pressure yielded crude product which was further purified by flash column chromatography on silica gel (0-80% EtOAc/hexanes) to afford 55 mg of Compound 31. This material was dissolved in about 6 mL Et₂O and HCl (g) was bubbled through the solution. A white precipitate formed immediately which was filtered and air dried to give Compound 31 hydrochloride.

¹H NMR (CD₃OD, 400 MHz): δ 7.32-7.63 (m, 14H), 6.81 (d, J=16 Hz, 1H), 6.22-6.30 (m, 1H), 4.45 (q, J=6.8 Hz, 1H) 3.59-3.78 (m, 2H), 1.71(d, J=6.8 Hz, 3H)

ESI(MS) of [C₂₃H₂₃N+H] calculated is 314.18, found 314.2

### Example 31

### (R,E)-3-(naphthalen-1-yl)-N-(1-phenylethyl)prop-2-en-1-amine (Compound 32)

The synthesis of the title compound was achieved in two steps:

### Step 1: (E)-3-(naphthalen-1-yl)acrylaldehyde

1-naphthaldehyde (0.285 g, 1.83 mmol) was dissolved in THF followed by the addition of 2-(triphenylphosphoranylidene) acetaldehyde (1.13 g, 3.66 mmol). This reaction mixture was refluxed overnight under a nitrogen atmosphere. The resulting orange-red reaction mixture was loaded directly on a silica gel column and eluted with 100% Et₂O. Rotary evaporation of the pooled product fractions gave a crude yellow oil which was further purified by chromatography on silica gel using 0-15% EtOAc/hexanes to give (E)-3-(naphthalen-1-yl)acrylaldehyde.

### Step 2: (R,E)-3-(naphthalen-1-yl)-N-(1-phenylethyl)prop-2-en-1-amine

To a RB flask containing 0.065 g (0.539 mmol) of (R)-1-phenylethanamine was added 5 mL of MeOH, 0.089 g (0.4928 mmol) of (E)-3-(naphthalen-1-yl)acrylaldehyde and 7 mg of phosphotungstic acid hydrate. The reaction mixture was stirred vigorously for 10 minutes before adding 19 mg (0.4902 mmol) of NaBH₄. The reaction mixture turned olive green with evolution of gas after which the color faded to light orange in about 20 minutes. The reaction mixture was diluted with 1:1 EtOAc/hexanes, washed with saturated NaHCO₃, water and brine, and dried over MgSO₄. Rotary evaporation under reduced pressure gave the crude product which was purified by flash column chromatography on silica gel (0-80% EtOAc/hexanes) to afford 70 mg of Compound 32.

Compound 32 was dissolved in 6 mL Et₂O and HCl (g) was bubbled through the solution. A white precipitate formed immediately which was filtered and air dried to give 30 mg of Compound 32 hydrochloride.

¹H NMR (CD₃OD, 400 MHz): δ8.11 (d, J=7.6 Hz, 1H), 7.85-7.90 (m, 2H), 7.45-7.67 (m, 10H), 6.22-6.29 (m, 1H), 4.50 (q, J=6.8 Hz, 1H) 3.71-3.90 (m, 2H), 1.73 (d, J=6.8 Hz, 3H) ESI(MS) of [C₂₁H₂₁N+H] calculated is 288.17, found 288.2

### Example 32

### (R,E)-3-(naphthalen-2-yl)-N-(1-phenylethyl)prop-2-en-1-amine (Compound 33)

The synthesis of the title compound was achieved in two steps:

### Step 1: (E)-3-(naphthalen-2-yl)acrylaldehyde

(E)-3-(naphthalen-2-yl)acrylaldehyde was obtained following the general procedure of Example 31, Step 1 using 1 equiv. of 2-naphthaldehyde and 2 equiv. of (triphenylphosphoranylidene)acetaldehyde.

### Step 2: (R,E)-3-(naphthalen-2-yl)-N-(1-phenylethyl)prop-2-en-1-amine

To a RB flask containing 0.067 g (0.554 mmol) of (R)-1-phenylethanamine was added 5 mL of MeOH, 0.084 g (0.5045 mmol) of (E)-3-(naphthalen-2-yl)acrylaldehyde and 7 mg of phosphotungstic acid hydrate. The reaction mixture was stirred vigorously for 10 minutes before adding 19 mg (0.4902 mmol) of NaBH₄. The reaction mixture turned dark blue with evolution of gas after which the color faded to light green in about 45 minutes. The reaction mixture was diluted with 1:1 EtOAc/hexanes, washed with saturated NaHCO₃, water and brine, and dried over MgSO₄. Rotary evaporation under reduced pressure provided crude product which was purified by flash column chromatography on silica gel (0-60% EtOAc/hexanes) to afford 52 mg of Compound 33. Compound 33 was dissolved in 6 mL Et₂O and HCl (g) was bubbled through the solution. A white precipitate formed immediately which was filtered and air dried to give 20 mg of Compound 33 hydrochloride.

¹H NMR (CD₃OD, 400 MHz): δ 7.47-7.85 (m, 12H), 6.94 (d, J=16 Hz, 1H), 6.30-6.38 (m, 1H), 4.46 (q, J=6.8 Hz, 1H) 3.63-3.81 (m, 2H), 1.71(d, J=6.8 Hz, 3H)

ESI(MS) of [C₂H₂₁N+H] calculated is 288.17, found 288.2

### Example 33

### (R,E)-N-(1-(naphthalen-1-yl)ethyl)-3-phenylprop-2-en-1-amine (Compound 34)

To a RB flask containing 0.200 g (1.16 mmol) of (R)-1-(naphthalen-1-yl)ethanamine was added 5 mL of MeOH, 0.140 g (1.0628 mmol) of trans-cinnamaldehyde and 15 mg of phosphotungstic acid hydrate. The reaction mixture was stirred vigorously for 10 minutes before adding 19 mg (0.4902 mmol) of NaBH₄. The reaction mixture turned dark blue with evolution of gas after which the color faded to whitish yellow in about 30 minutes. The reaction mixture was diluted with 1:1 EtOAc/hexanes, washed with saturated NaHCO₃, water and brine, and dried over MgSO₄. Rotary evaporation under reduced pressure yielded a gummy solid. This material was purified by flash column chromatography on silica gel (0-75% EtOAc/hexanes) to afford 270 mg of Compound 34.

Compound 34 was dissolved in 10 mL Et₂O and HCl (g) was bubbled through the solution. A white precipitate formed immediately which was filtered and air dried to give 238 mg of Compound 34 hydrochloride.

¹H NMR (CD₃OD, 400 MHz): δ 8.6 (d, J=7.6 Hz, 1H), 7.98-8.00 (m, 2H), 7.73 (d, J=7.6 Hz, 1H), 7.57-7.65 (m, 3H), 7.29-7.40 (m, 5H), 6.69 (d, J=16 Hz, 1H), 6.20-6.28 (m, 1H), 5.42 (q, J=6.8 Hz, 1H), 3.71-3.86 (m, 2H), 1.82 (d, J=6.8 Hz, 3H)

ESI(MS) of [C₂₁H₂₁N+H] calculated is 288.17, found 288.2

### Example 34

### (R,E)-N-(1-(naphthalen-2-yl)ethyl)-3-phenylprop-2-en-1-amine (Compound 35)

Compound 35 was obtained following then general procedure of Example 33 using 0.200 g (1.169 mmol) of (R)-1-(naphthalen-2-yl)ethanamine and 0.140 g (1.0628 mmol) of trans-cinnamaldehyde.

¹H NMR (CD₃OD, 400 MHz): δ 7.99-8.03 (m, 2H), 7.91-7.94 (m, 2H), 7.56-7.59 (m, 2H), 7.41 (d, J=7.6 Hz, 1H), 7.29-7.35 (m, 3H), 6.75 (d, J=16 Hz, 1H), 6.19-6.26 (m, 1H), 4.62 (q, J=6.8 Hz, 1H), 3.60-3.80 (m, 2H), 1.79 (d, J=6.8 Hz, 3H)

ESI(MS) of [C₂₁H₂₁N+H] calculated is 288.17, found 288.2

### Example 35

### (R,E)-3-cyclohexyl-N-(1-phenylethyl)prop-2-en-1-amine (Compound 36)

Compound 36 was made following the general procedure of Example 33 using 0.197 g (1.62 mmol) of (R)-1-phenylethanamine and 0.205 g (1.48 mmol) of (E)-3-cyclohexylacrylaldehyde to give Compound 36 hydrochloride.

### Example 36

### (R,E)-2-phenyl-N-(1-phenylethyl)but-2-en-1-amine (Compound 37)

Compound 37 was made following the general procedure of Example 33 using 0.254 g (2.1001 mmol) of (R)-1-phenylethanamine and 0.279 g (1.909 mmol) of (E)-2-phenylbut-2-enal to give 100 mg of Compound 37 hydrochloride .

¹H NMR (CD₃OD, 400 MHz): δ 7.34-7.43 (m, 10H), 7.22-7.27 (m, 2H), 5.97 (q, J=6.8 Hz, 1H), 4.34 (q, J=6.8 Hz, 1H), 3.77 (dd, J=44 and 13.6 Hz, 2H), 1.66 (d, J=6.8 Hz, 1H), 1.61 (d, J=6.8 Hz, 3H)

ESI(MS) of [C₁₈H₂₁N+H] calculated is 252.17, found 252.2

### Example 37

### (R)—N-(cyclohexylmethyl)-1-phenylethanamine (Compound 38)

Compound 38 was made following the general procedure of Example 33 using 0.222 g (1.83 mmol) of (R)-1-phenylethanamine and 0.187 g (1.66 mmol) of cyclohexane carboxyaldehyde to give 160 mg of Compound 38 hydrochloride.

¹H NMR (CD₃OD, 400 MHz): δ 7.47 (s, 5H), 4.36 (q, J=6.8 Hz, 1H), 2.79 (dd, J=12.4 and 6.8 Hz, 1H), 2.55 (dd, J=12.4 and 7.2 Hz, 1H), 1.67-1.73 (m, 8H), 0.98-1.3 (m, 3H)

ESI(MS) of [C₁₅H₂₃N+H] calculated is 218.18, found 218.2

### Example 38

### (S)-methyl 2-(cinnamylamino)-2-phenylacetate (Compound 39)

A RB flask was charged with 8-10 mL DCM, (S)-phenylglycine methyl ester hydrochloride (0.422 g, 2.09 mmol) and phosphotungstic acid hydrate (27 mg). The flask, was purged with nitrogen and (0.251 g, 1.905 mmol) of trans-cinnamaldehyde was added via a syringe. The reaction mixture was stirred for 10 minutes before adding (0.605 g, 2.85 mmol) of NaBH(OAc)₃. After stirring overnight at rt, the reaction was worked up by addition of 10% NaOH solution followed by EtOAc extraction. The EtOAc layer was washed with brine, dried over MgSO₄ and condensed by rotary evaporation under reduced pressure to give an oil. The oil was purified by flash column chromatography on 70 g of silica gel (30% EtOAc/hexanes) to afford 225 mg (38%) of Compound 39. This material was dissolved in 10 mL of Et₂O and this solution was treated with a slow stream of HCl(g). The mother liquor was decanted from the white gummy glass that had formed. This glass crystallized upon Et₂O trituration and standing at rt overnight to yield Compound 39 hydrochloride.

¹H NMR (CD₃OD, 400 MHz): δ 7.29-7.54 (m, 10H), 6.81 (d, J=16 Hz, 1H), 6.21-6.28 (m, 1H), 5.24 (s, 1H), 3.71-3.83 (m, 5H)

ESI(MS) of [C₁₈H₁₉NO₂+H] calculated is 282.14, found 282.2

### Example 39

### (R)-methyl 2-(cinnamylamino)-2-phenylacetate (Compound 40)

Compound 40 was obtained following the general procedure of Example 38 using (R)-phenylglycine methyl ester hydrochloride (0.434 g, 2.159 mmol) and trans-cinnamaldehyde (0.259 g, 1.96 mmol) to afford 180 mg of Compound 40 hydrochloride.

¹H NMR (CD₃OD, 400 MHz): δ 7.29-7.52 (m, 10H), 6.81 (d, J=16 Hz, 1H), 6.20-6.28 (m, 1H), 5.24 (s, 1H), 3.71-3.83 (m, 5H)

ESI(MS) of [C₁₈H₁₉NO₂+H] calculated is 282.14, found 282.2

### Example 40

### (R)-methyl 2-((3,3-diphenylallyl)amino)-2-phenylacetate (Compound 42)

To a solution of 3-phenyl cinnamaldehyde (0.224 g, 1.017 mmol) in 8 mL DCE under a nitrogen atmosphere was added (R)-phenylglycine methyl ester hydrochloride (0.238 g, 1.182 mmol), phosphotungstic acid hydrate (15 mg,) and NaBH(OAc)₃ (0.320 g, 1.511 mmol). The reaction was stirred at rt overnight and worked up by adding 10% NaOH solution and extracting with EtOAc. The EtOAc layer was washed with brine, dried over MgSO₄ and rotary evaporated under reduced pressure to give an oil. The oil was purified by flash column chromatography on 80 g of silica gel (30% EtOAc/hexanes) to afford 136 mg of Compound 42. This material was dissolved in 10 mL of Et₂O and treated with HCl(g). Slow addition of hexanes resulted in a milky mixture which upon standing overnight at rt yielded Compound 42 hydrochloride.

¹H NMR (CD₃OD, 400 MHz): δ 7.42-7.44 (m, 3H), 7.37-7.40 (m, 5H), 7.31-7.33 (m, 3H), 7.23-7.26 (m, 2H), 7.09-7.12 (m, 2H), 6.12 (t, J=6.8 Hz, 1H), 5.15 (s, 1H), 3.71-3.75 (m, 5H)

ESI(MS) of [C₂₃H₂₂NO₂+H] calculated is 358.17, found 358.2

### Example 41

### (S)-methyl 2-((3,3-diphenylallyl)amino)-2-phenylacetate (Compound 43)

Compound 43 was obtained following the general procedure of Example 40 using (S)-phenylglycine methyl ester hydrochloride (0.240 g, 1.193 mmol) and 3-phenyl cinnamaldehyde (0.226 g, 1.08 mmol) to afford Compound 43 hydrochloride.

¹H NMR (CD₃OD, 400 MHz): δ 7.42-7.48 (m, 3H), 7.37-7.40 (m, 5H), 7.31-7.33 (m, 3H), 7.23-7.26 (m, 2H), 7.09-7.12 (m, 2H), 6.12 (t, J=6.8 Hz, 1H), 5.15 (s, 1H), 3.72-3.75 (m, 5H)

ESI(MS) of [C₂₃H₂₂NO₂+H] calculated is 358.17, found 358.2

### Example 42

### (R)—N-(1-(4-bromophenyl)ethyl)-3,3-diphenylprop-2-en-1-amine (Compound 44)

Compound 44 was obtained following the general procedure of Example 25 using 0.183 g (0.9156 mmol) of (R)-1-phenylethanamine and 0.173 g (0.832 mmol) of 3-phenyl cinnamaldehyde to give 80 mg of Compound 44.

Compound 44 was dissolved in 10 mL Et₂O and HCl (g) was bubbled through the solution. The white ppt which formed upon trituration with 10% hexane/ethyl acetate was isolated by vacuum filtration and air drying to yield Compound 44 hydrochloride.

¹H NMR (CD₃OD, 400 MHz): δ 7.53-7.55 (m, 2H), 7.23-7.36 (m, 10H), 7.03 (m, 2H), 6.09 (t, J=6.4 Hz, 1H), 4.37 (q, J=6.8 Hz, 1H), 3.51-3.63 (m, 2H), 1.60 (d, J=6.4 Hz, 3H)

ESI(MS) of [C₂₃H₂₂BrN+H] calculated is 392.09, found 392.1

### Example 43

### (R)—N-(1-(4-fluorophenyl)ethyl)-3,3-diphenylprop-2-en-1-amine (Compound 45)

Compound 45 was obtained following the general procedure of Example 25 using 0.194 g (1.395 mmol) of (R)-1-phenylethanamine and 0.264 g (1.26 mmol) of 3-phenyl cinnamaldehyde to give 90 mg of Compound 45.

Compound 45 was dissolved in 10 mL Et₂O and HCl (g) was bubbled through the solution. The white ppt formed which upon trituration with 10% hexane/ethyl acetate was isolated by vacuum filtration and air drying to yield Compound 45 hydrochloride.

¹H NMR (CD₃OD, 400 MHz): δ 7.05-7.36 (m, 14H), 6.10 (t, J=6.8 Hz, 1H), 4.40 (q, J=6.8 Hz, 1H), 3.52-3.64 (m, 2H), 1.61 (d, J=6.4 Hz, 3H)

ESI(MS) of [C₂₃H₂₂FN+H] calculated is 332.17, found 332.2

### Example 44

### (R)-3,3-diphenyl-N-(1-phenylpropyl)prop-2-en-1-amine (Compound 46)

Compound 46 was obtained following the general procedure of Example 25 using 0.194 g (1.395 mmol) of (R)-1-phenylpropan-1-amine and 0.305 g (1.46 mmol) of 3-phenyl cinnamaldehyde to give 400 mg of Compound 46.

Compound 46 was dissolved in 6 mL Et₂O and HCl (g) was bubbled through the solution. A white ppt immediately formed which was filtered, vacuum and air dried to give 340 mg of Compound 46 hydrochloride.

¹H NMR (CD₃OD, 400 MHz): 7.21-7.40 (m, 13H), 7.02-7.42 (m, 2H), 6.10 (t, J=6.8 Hz), 4.08 (m, 1H), 3.48-3.62 (m, 1H), 1.91-2.11 (m, 2H), 0.78 (t, J=7.2 Hz, 3H)

ESI(MS) of [C₂₄H₂₅N+H] calculated is 328.20, found 328.2

### Example 45

### (R)-4-(1-((3,3-diphenylallyl)nitro)ethyl)aniline (Compound 47)

To a RB flask under a nitrogen atmosphere was added 3-phenyl-cinnamaldehyde (0.483 g, 2.319 mmol), 8 mL DCE, (R)-1-(4-nitrophenyl)ethanamine hydrochloride (0.517 g, 2.55 mmol) and phosphotungstic acid hydrate (33 mg). The reaction was stirred at rt for 5 minutes before addition of NaBH(OAc)₃ (1.081 g, 5.1025 mmol). The reaction was then stirred at rt overnight and worked up by addition of 10% NaOH solution and EtOAc extraction. The EtOAc layer was washed with brine, dried over MgSO₄ and rotary evaporated under reduced pressure to give a jade colored crude oil. The crude oil was further purified by flash column chromatography on 200 g of silica gel (30-70% EtOAc/hexanes) to afford Compound 47.

¹H NMR (CD₃Cl, 400 MHz): δ 8.102 (d, J=8.8 Hz, 2H), 7.38 (d, J=8.8 Hz, 2H), 7.18-7.29 (m, 14H), 7.05-7.08 (m, 2H), 6.14 (t, J=7.2 Hz, 1H), 3.86 (q, J=6.4 Hz, 1H), 3.10-3.19 (m, 2H), 1.31 (d, J=6.4 Hz, 3H)

ESI(MS) of [C₂₃H₂₂N₂O₂+H] calculated is 359.17, found 359.2

### Example 46

### (R)-4-(1-((3,3-diphenylallyl)amino)ethyl)aniline (Compound 48)

To a stirred mixture of zinc dust and 10 mL of MeOH was added 5 drops of 2N HCl. After 2 minutes the excess HCl was neutralized with 1.5 mL of NH₄OH. A solution of (R)-4-(1-((3,3-diphenylallyl)nitro)ethyl)aniline (0.063 g, 0.176 mmol) and NH₄HCO₂ (0.172 g, 2.727 mmol) in MeOH was added dropwise. The reaction mixture was stirred vigorously at rt for 10 minutes and worked up by, filtering through a plug of celite, and rotary evaporation in vacuo to yield a white solid. This material was mixed with NH₄OH (15 mL) and Et₂O (25 mL). The ethereal layer was separated, dried over MgSO₄ and rotary evaporated in vacuo to give 43 mg (75%) of Compound 48.

¹H NMR (CD₃Cl, 400 MHz): δ 7.22-7.32 (m, 10H), 7.10 (m, 2H), 7.02(d, J=8.4 Hz), 6.61 (d, J=8.4 Hz, 2H), 6.16 (t, J=7.2 Hz, 1H), 3.66 (q, J=6.4 Hz, 1H), 3.19 (d, J=6.8 Hz, 2H), 1.28 (d, J=6.4 Hz, 3H)

### Example 47

### (R,Z)-3-(4-nitrophenyl)-3-phenyl-N-(1-phenylethyl)prop-2-en-1-amine (Compound 49)

The title compound was synthesized in a total of 5 steps:

### Step 1: (Z)-ethyl 3-(4-nitrophenyl)-3-phenylacrylate

To a RB flask containing 60% NaH (0.356 g, 8.904 mmol) in 15 mL anhydrous THF was slowly added triethyl-phosphonoacetate (1.892 g, 8.44 mmol). The reaction was stirred at rt for 30 minutes after which a solution of 4-nitrobenzophenone (1.5 g, 6.595 mmol) in 15 mL THF was added. This mixture was gently refluxed overnight. The reaction was worked up by quenching with a 1:1 EtOAc/water. The aqueous layer was re-extracted with EtOAc and the combined organic layers were washed with brine, dried over MgSO₄ and rotary evaporated under reduced pressure to give a yellow oil. This material was purified by flash column chromatography on 300 g of silica gel (0-5-10-15% EtOAc/hexanes) to afford (Z)-ethyl 3-(4-nitrophenyl)-3-phenylacrylate.

¹H NMR (CD₃Cl, 400 MHz): δ 8.27 (m, 2H), 7.24-7.40 (m, 7H), 6.48 (s, 1H), 4.07 (q, J=7.2 Hz, 2H), 1.28 (t, J=7.2 Hz, 3H)

### Step 2: (Z)-3-(4-nitrophenyl)-3-phenylacrylic acid

1 equiv. of (Z)-ethyl 3-(4-nitrophenyl)-3-phenylacrylate (1.4 g, 4.7088 mmol) was mixed with 10-15 mL MeOH. To this mixture was added 6.5 equiv. of 2M solution of K₂CO₃ in MeOH (˜14 mL) and the mixture was gently refluxed for 1 hour. The reaction became yellow colored and homogenous. The majority of the MeOH was removed by rotary evaporation. The residual material was partitioned between hexane and water. The aqueous layer was acidified with 2N HCl to afford a white ppt which was isolated by vacuum filtration and air dried to yield 1.1 g (86%) of (Z)-3-(4-nitrophenyl)-3-phenylacrylic acid.

¹H NMR (CD₃Cl, 400 MHz): δ 8.25 (d, J=8.4 Hz, 2H), 7.34-7.42 (m, 5H), 7.22-7.24 (m, 2H), 6.46 (s, 1H)

### Step 3: (Z)-3-(4-nitrophenyl)-3-phenylprop-2-en-1-ol

(Z)-3-(4-nitrophenyl)-3-phenylacrylic acid (0.052 g, 0.193 mmol, 1 equiv.) was dissolved in 4 ml of THF and 1.5 equiv. of 1M BH₃-THF was added slowly under an atmosphere of nitrogen. After stirring at rt for 30 minutes, the reaction was worked up by addition of saturated NaHCO₃ and EtOAc. The EtOAc layer was washed with brine, dried over MgSO₄ and rotary evaporated to give an oil which was further purified by flash column chromatography on 20 g of silica gel (30-70% EtOAc/hexanes) to afford 30 mg (61%) of (Z)-3-(4-nitrophenyl)-3-phenylprop-2-en-1-ol.

¹H NMR (CD₃Cl, 400 MHz): δ 8.25 (d, J=8.4 Hz, 2H), 7.38 (d, J=8.4 Hz, 2H), 7.30-7.32 (m, 3H), 7.19-7.21 (m, 2H), 6.35 (t, J=6.8 Hz, 1H), 4.207 (d, J=6.8 Hz, 2H)

### Step 4: (Z)-3-(4-nitrophenyl)-3-phenylacrylaldehyde

Under an atmosphere of nitrogen, a solution of (Z)-3-(4-nitrophenyl)-3-phenylprop-2-en-1-ol (0.560 g, 2.194 mmol) in DCM (3 mL) was added slowly to a milky white solution of Dess-Martin periodinane (1.39 g, 3.29 mmol) in DCM (10 mL). The reaction mixture was stirred at rt overnight. The reaction was quenched by the addition of 1:1 (2N NaOH:water), stirring for 10 minutes and extracting twice with Et₂O. The combined Et₂O extracts were washed with water, brine, dried over MgSO₄ and rotary evaporated to yield (Z)-3-(4-nitrophenyl)-3-phenylacrylaldehyde (0.465 mg, 83%).

¹H NMR (CD₃Cl, 400 MHz): δ 9.50 (d, J=8 Hz, 1H), 8.33-8.35 (m, 2H), 7.48-7.52 (m, 2H), 7.29-7.48 (m, 5H), 6.73 (d, J=8.4 Hz, 2H)

### Step 5: (R,Z)-3-(4-nitrophenyl)-3-phenyl-N-(1-phenylethyl)prop-2-en-1-amine (Compound 49)

A RB flask under a nitrogen atmosphere was charged with (Z)-3-(4-nitrophenyl)-3-phenylacrylaldehyde (0.465 g, 1.84 mmol), 4 mL DCE, a solution of (R)-1-phenylethanamine (0.244 g, 2.02 mmol) in 3 mL DCE, and phosphotungstic acid hydrate (26 mg). The reaction was stirred at rt for 5 minutes before addition of NaBH(OAc)₃ (0.856 g, 4.04 mmol). The reaction was then stirred at rt overnight and worked up by addition of 10% NaOH solution and extraction with EtOAc. The EtOAc layer was washed with brine, dried over MgSO₄ and rotary evaporated under reduced pressure to give a yellow colored oil. The crude oil was purified by flash column chromatography on 150 g of silica gel (30-70% EtOAc/hexanes) to afford 440 mg (67%) Compound 49.

¹H NMR (CD₃Cl, 400 MHz): δ 8.13 (d, J=6.8 Hz, 2H), 7.12-7.29 (m, 10H), 6.25 (t, J=7.2 Hz, 1H), 3.74 (q, J=6.8 Hz, 1H), 3.16 (d, J=7.2 Hz, 2H), 1.34 (d, J=6.4 Hz, 3H)

### Example 48

### (R,Z)-4-(1-phenyl-3-((1-phenylethyl)amino)prop-1-en-1-yl)aniline (Compound 50)

A RB flask was charged with zinc (2 g, 1.25 mmol) and 20 mL of MeOH. To this was added 15 drops of 2N HCl and the mixture stirred for 5 minutes. The HCl was then neutralized with excess of NH₄OH solution. A solution of Compound 49 (0.447 g, 1.25 mmol) and NH₄HCO₂ (1.212 g, 19.22 mmol) in MeOH was added dropwise. The reaction mixture was stirred vigorously at room temperature for 10 minutes and then worked up by filtering through a plug of celite and rotary evaporation in vacuo to give a yellow solid. This material was taken up in a mixture of NH₄OH (15 mL) and Et₂O (35 mL). The ethereal layer was separated, dried over MgSO₄ and rotary evaporated in vacuo to yield 294 mg (73%) of Compound 50.

### Example 49

### (R,E)-3-(4-nitrophenyl)-3-phenyl-N-(1-phenylethyl)prop-2-en-1-amine (Compound 55)

The title compound was synthesized in a total of 5 steps:

### Step 1: (E)-ethyl3-(4-nitrophenyl)-3-phenylacrylate

To a RB flask containing 60% NaH (0.506 g, 12.66 mmol) in 40 mL anhydrous THF was slowly added triethyl-phosphonoacetate (2.836 g. 12.05 mmol). The reaction was stirred at rt for 30 minutes after which a solution of 4-nitrobenzophenone (2.14 g, 9.41 mmol) in 20 mL THF was added. This mixture was gently refluxed overnight. The reaction was worked up by quenching with a 1:1 EtOAc/water. The aqueous layer was re-extracted with EtOAc. The combined organic layers were washed with brine, dried over MgSO₄ and rotary evaporated under reduced pressure to give the crude product as a yellow-colored oil. This material was purified by flash column chromatography on 300 g of silica gel (0-5-10-15% EtOAc/hexanes) and the mixed fractions combined and rechromatographed to afford (E)-ethyl 3-(4-nitrophenyl)-3-phenylacrylate.

### Step 2: (E)-3-(4-nitrophenyl)-3-phenylacrylic acid

(E)-ethyl 3-(4-nitrophenyl)-3-phenylacrylate (1.67 g, 5.617 mmol) was mixed with 25 mL MeOH. To this mixture was added a solution composed of 30 mL of water and 6.2 g of K₂CO₃ and 2.0 g of NaOH. This mixture was gently refluxed until thin layer analysis indicated complete reaction. The reaction became yellow colored and homogenous. The majority of the MeOH was removed by rotary evaporation and the residual material was partitioned between hexane and water. The aqueous layer was acidified with 2N HCl to afford a white ppt which was isolated by vacuum filtration and air dried to yield 1.41 g (93%) of (E)-3-(4-nitrophenyl)-3-phenylacrylic acid.

### Step 3: (Z)-3-(4-nitrophenyl)-3-phenylprop-2-en-1-ol

(E)-3-(4-nitrophenyl)-3-phenylacrylic acid (0.597 g, 2.23 mmol) was dissolved in 15 mL of EtOAc and 5.7 mL of 1M BH₃-THF was added slowly under an atmosphere of nitrogen. Brisk effervescence followed by the formation of a heavy ppt. After stirring at rt for several days the ppt disappeared and TLC analysis indicated complete reaction. The reaction was worked up by addition of excess 2N HCl and extraction with EtOAc/hexane (1:1). The extract was washed with saturated NaHCO₃ and EtOAc and then dried over NaSO₄. Rotary evaporation gave an orange-brown colored oil which was further purified by flash column chromatography on 130 g of silica gel (20-70% EtOAc/hexanes) to afford the 309 mg (54%) of (E)-3-(4-nitrophenyl)-3-phenylprop-2-en-1-ol.

### Step 4: (E)-3-(4-nitrophenyl)-3-phenylacrylaldehyde

Under an atmosphere of nitrogen, a solution of (E)-3-(4-nitrophenyl)-3-phenylprop-2-en-1-ol (0.305 g, 1.19 mmol) in DCM (5 mL) was added slowly to a milky white solution of Dess-Martin periodinane 756 mg, 1.78 mmol) in DCM (10 mL). The reaction mixture was stirred at rt overnight. The reaction was quenched by the addition of 1:1 (2N NaOH:water), stirring for 10 minutes and extracting twice with Et₂O. The combined Et₂O extracts were washed with water, brine, dried over MgSO₄ and rotary evaporated to yield (E)-3-(4-nitrophenyl)-3-phenylacrylaldehyde (276 mg, 91%).

### Step 5: (R,E)-3-(4-nitrophenyl)-3-phenyl-N-(1-phenylethyl)prop-2-en-1-amine (Compound 55)

A RB flask under a nitrogen atmosphere was charged with (E)-3-(4-nitrophenyl)-3-phenylacrylaldehyde (0.269 g, 1.06 mmol), 15 mL DCE, (R)-1-phenylethanamine (0.156 g, 1.29 mmol), and phosphotungstic acid hydrate (25 mg). The reaction was stirred at rt for 5 minutes before addition of NaBH(OAc)₃ (0.506 g, 2.38 mmol). The reaction was then stirred at rt overnight and worked up by addition of 10% NaOH solution and extraction with Et₂O. The Et₂O layer was washed with brine, dried over MgSO₄ and rotary evaporated under reduced pressure to give the product as a yellow colored oil. The crude oil was purified by flash column chromatography on 150 g of silica gel (20-90% EtOAc/hexanes) to afford 251 mg Compound 55.

ESI(MS) of [C₂₃H₂₂N₂O₂+H] calculated is 359.18, found 359.2

### Example 50

### (R,E)-4-(1-phenyl-3-((1-phenylethyl)amino)prop-1-en-1-yl)aniline (Compound 56)

A RB flask was charged with excess zinc dust (1.35 g) and 20 mL of MeOH. To this was added 5 drops of 2N HCl and the mixture stirred at rt. After approximately 10 minutes, 1.5 mL of NH₄OH solution was added. This was followed by the dropwise addition of a solution of (R,E)-3-(4-nitrophenyl)-3-phenyl-N-(1-phenylethyl)prop-2-en-1-amine (0.081 g, 0.22 mmol) and NH₄HCO₂ (0.422 g, 6.69 mmol) in MeOH (15 mL). The reaction mixture was stirred vigorously at rt for 10 minutes and then worked up by filtering through a plug of celite and rotary evaporation. The residual material was taken up in a mixture of NH₄OH (15 mL) and Et₂O (35 mL). The ethereal layer was separated, dried over MgSO₄ and rotary evaporated in vacuo to yield 75 mg Compound 56.

ESI(MS) of [C₂₃H₂₄N₂+H] calculated is 329.20, found 329.2

### Example 51

### (R)-methyl 2-((3,3-diphenylallyl)amino)-2-(4-fluorophenyl)acetate (Compound 57)

A RB flask under a nitrogen atmosphere was charged with methyl (D)-2-(4-fluorophenyl)glycinate (0.205 g, 1.12 mmol), 10 mL DCE, β-phenylcinnamaldehyde (0.238 g, 1.14 mmol), and phosphotungstic acid hydrate (25 mg). The reaction was stirred at rt for 5 minutes before addition of NaBH(OAc)₃ (0.466 g, 2.2 mmol). The reaction was then stirred at rt overnight and worked up by addition of 10% NaOH solution and extraction with Et₂O. The Et₂O layer was washed with brine, dried over MgSO₄ and rotary evaporated under reduced pressure to give the product as an oil. The crude oil was purified by flash column chromatography on 35 g of silica gel (10% EtOAc/hexanes) to afford 148 mg Compound 57.

ESI(MS) of [C₂₄H₂₂FNO₂+H] calculated is 376.17, found 376.2

### Example 52

### (R)—N-(2 (5H-dibenzo[a,d][7]annulen-5-ylidene)ethyl)-1-(4-fluorophenyl) ethanamine (Compound 58)

The title compound was synthesized in 4 steps.

### Step 1: ethyl 2-(5H-dibenzo[a,d][7]annulen-5-ylidene)acetate

To a RB flask, under a nitrogen atmosphere, was added 350 mg (8.75 mmol) of 60% NaH/mineral oil and 20 mL of dry THF. To the stirred reaction a solution of triethyl phosphonoacetate (1.96 g, 8.75 mmol) in 5 mL THF was added dropwise such that the off-gas and exotherm were controlled. This was followed by the dropwise addition of a solution of 5-dibenzosuberenone (1.44 g, 6.98 mmol) in 5 mL of THF. The reaction was stirred and heated to gentle reflux overnight. The reaction was worked up by addition of water and sat NaCl solution, hexane extraction, drying (MgSO₄), filtration and then condensation by rotary evaporation yielded 2.186 g of oil. This oil was purified by flash column chromatography on 35 g silica gel (0-5% Et₂O/hexanes) to afford 1.96 g ethyl 2-(5H-dibenzo[a,d][7]annulen-5-ylidene)acetate.

### Step 2: 2-(5H-dibenzo[a,d][7]annulen-5-ylidene)ethanol

A RB flask was charged with ethyl 2-(5H-dibenzo[a,d][7]annulen-5-ylidene)acetate (246 mg, 0.89 mmol), 15 mL of hexane and 5 mL of DCM. The flask was sealed with a rubber septum, flushed with nitrogen, and cooled in a dry ice/acetone bath. To the cold reaction was added dropwise 1M DIBAL-H/hexanes solution (2.0 mL, 2.0 mmol). After 1 hr the cooling bath was allowed to dissipate. While still at a sub-zero temperature, the reaction was quenched by the addition of 2N HCl and the product isolated by extraction with 20% EtOAc/hexane, drying (NaCl/MgSO₄) and rotary evaporation to yield 2-(5H-dibenzo[a,d][7]annulen-5-ylidene)ethanol as an oil. The product was used without further purification in Step 3.

### Step 3: 2-(5H-dibenzo[a,d][7]annulen-5-ylidene)acetaldehyde

The 2-(5H-dibenzo[a,d][7]annulen-5-ylidene)ethanol from Step 2 was dissolved in 10 mL DCM and added dropwise to a mixture of Dess-Martin periodinane (563 mg, 1.33 mmol) in 10 mL of DCM. The flask was sealed and stirred at rt overnight. After this time the reaction product was isolated by extraction (20% Et₂O/hexane) from excess dilute NaOH. The organic extract was dried (NaCl) and condensed by rotary evaporation to yield an oil. This material was further purified by silica gel (15 g) chromatography eluting with 10-15% Et₂O/hexane to yield 175 mg of 2-(5H-dibenzo[a,d][7]annulen-5-ylidene)acetaldehyde as an oil.

### Step 4: (R)—N-(2 (5Hdibenzo[a,d][7]annulen-5-ylidene)ethyl)-1-(4-fluorophenyl)ethanamine (Compound 58)

A RB flask under a nitrogen atmosphere was charged with 2-(5H-dibenzo[a,d][7]annulen-5-ylidene)acetaldehyde (0.175 g, 0.52 mmol), 10 mL DCE, (R)-1-(4-fluorophenyl)ethanamine (0.073 g, 0.52 mmol), and phosphotungstic acid hydrate (20 mg). The reaction was stirred at rt for 10 minutes before addition of NaBH(OAc)₃ (0.222 g, 1.0 mmol). The reaction was then stirred at rt overnight and worked up by addition of excess dilute NaOH solution and extraction with 20% EtOAc/hexane. The organic layer was dried over MgSO₄ and condensed by rotary evaporation under reduced pressure to give the product as an oil. The oil was purified by column chromatography over 15 g of silica gel (20-40% EtOAc/hexanes) to afford 163 mg Compound 58.

ESI(MS) of [C₂₅H₂₂FN+H] calculated is 356.18, found 356.19

### Example 53

### (R)—N-(1-(4-chlorophenyl)ethyl)-3,3-diphenylprop-2-en-1-amine (Compound 59)

A RB flask under a nitrogen atmosphere was charged with (R)-1-(4-chlorophenyl)ethanamine (0.165 g, 1.06 mmol), 10 mL DCE, β-phenylcinnamaldehyde (0.226 g, 1.08 mmol), and phosphotungstic acid hydrate (15 mg). The reaction was stirred at rt for 20 minutes before addition of NaBH(OAc)₃ (0.324 g, 1.53 mmol). The reaction was then stirred at rt overnight and worked up by addition of excess dilute NaOH solution and extraction with 20% EtOAc/hexane. The organic layer was dried over NaCl/MgSO₄ and condensed by rotary evaporation under reduced pressure to give the product as an oil. The oil was purified by column chromatography over 15 g silica gel (10-50% EtOAc/hexanes) to afford 202 mg Compound 59.

ESI(MS) of [C₂₃H₂₂ClN+H] calculated is 348.15, found 348.2

### Example 54

### (R)-2-(10,11-dihydro-5H-dibenzo[a,d][7]annulen-5-ylidene)-N-(1-(4-fluorophenyl)ethyl)ethanamine (Compound 60)

The title compound was synthesized in 4 steps.

### Step 1: ethyl 2-(10,11-dihydro-5H-dibenzo[a,d][7]annulen-5-ylidene)acetate

To a RB flask, under a nitrogen atmosphere, was added 210 mg (5.25 mmol) of 60% NaH/mineral oil and 15 mL of dry THF. To the stirred reaction a solution of triethyl phosphonoacetate (0.913 g, 4.07 mmol) in 5 mL THF was added dropwise such that the off-gas and exotherm were controlled. This was followed by the dropwise addition of a solution of 5-dibenzosuberone (0.312 g, 1.50 mmol) in 5 mL of THF. The reaction was stirred and heated to gentle reflux overnight. The reaction was worked up by addition of excess 2N HCl and water, hexane extraction, drying (MgSO₄), filtration and then condensation by rotary evaporation yielded an oil. This oil was purified by flash column chromatography on 15 g of silica gel (0-5% Et₂O/hexanes) to afford 0.335 g ethyl 2-(10,11-dihydro-5H-dibenzo[a,d][7]annulen-5-ylidene)acetate.

### Step 2: 2-(10,11-dihydro-5H-dibenzo[a,d][7]annulen-5-ylidene)ethanol

A RB flask was charged with ethyl 2-(10,11-dihydro-5H-dibenzo[a,d][7]annulen-5-ylidene)acetate (330 mg, 1.18 mmol), 15 mL of hexane. The flask was sealed with a rubber septum, flushed with nitrogen, and cooled in a dry ice/acetone bath. To the cold reaction was added dropwise 1M DIBAL-H/hexanes solution (2.6 mL, 2.6 mmol). After 1 hr the cooling bath was allowed to dissipate. While still at a sub-zero temperature, the reaction was quenched by the addition of excess 2N HCl and the product isolated by extraction with 20% EtOAc/hexane, drying (NaCl/MgSO₄) and rotary evaporation to yield 333 mg of 2-(10,11-dihydro-5H-dibenzo[a,d][7]annulen-5-ylidene)ethanol as an oil which solidified to a white solid upon standing. The product was used without further purification in Step 3.

### Step 3: 2-(10,11-dihydro-5H-dibenzo[a,d][7]annulen-5-ylidene)acetaldehyde

The 2-(10,11-dihydro-5H-dibenzo[a,d][7]annulen-5-ylidene)ethanol from Step 2 was dissolved in 10 mL DCM and added dropwise to a mixture of Dess-Martin periodinane (751 mg, 1.77 mmol) in 10 mL of DCM. The flask was sealed and stirred at rt. After 2 hr, TLC analysis indicated complete reaction and the product was isolated by hexane extraction from excess dilute NaOH. The organic extract was washed with sat NaCl solution, dried (MgSO₄) and condensed by rotary evaporation to yield 293 mg of 2-(10,11-dihydro-5H-dibenzo[a,d][7]annulen-5-ylidene)acetaldehyde as an oil. This material was not further purified but used directly in step 4.

### Step 4: (R)-2-(10,11-dihydro-5H-dibenzo[a,d][7]annulen-5-ylidene)-N-(1-(4-fluorophenyl)ethyl)ethanamine (Compound 60)

A RB flask under a nitrogen atmosphere was charged with 2-(10,11-dihydro-5H-dibenzo[a,d][7]annulen-5-ylidene)acetaldehyde (0.287 g, 1.22 mmol), 10 mL DCE, (R)-1-(4-fluorophenyl)ethanamine (0.170 g, 1.22 mmol), and phosphotungstic acid hydrate (28 mg). The reaction was stirred at rt for 40 minutes before addition of NaBH(OAc)₃ (0.541 g, 2.44 mmol). The reaction was then stirred at rt overnight and worked up by addition of excess dilute NaOH solution and extraction with 20% EtOAc/hexane. The organic layer was dried over MgSO₄ and condensed by rotary evaporation under reduced pressure to give the product as an oil. The oil was further purified by column chromatography over 15 g of silica gel (20-40% EtOAc/hexanes) to give Compound 60.

ESI(MS) of [C₂₅H₂₄FN+H] calculated is 358.18, found 358.2

### Example 55

### (R)—N-(2-(9H-fluoren-9-ylidene)ethyl)-1-(4-fluorophenyl)ethanamine (Compound 61)

The title compound was synthesized by a 3-step synthetic sequence:

### Step 1: 2-(9H-Fluoren-9-ylidene)ethanol

A rubber stoppered RB flask, with a magnetic stir bar and under a positive nitrogen atmosphere, was charged with 9-fluorenylidene acetate (0.592 g, 2.36 mmol) and 35 mL of hexane. The flask was cooled in a dry ice-acetone bath and the contents reacted by the dropwise addition of 4 mL of 1M DIBAL-H/hexane. The reaction was vigorously stirred as the dry-ice/acetone cooling bath dissipated. While still cold, the reaction was worked up by quenching with excess 2N HCl. The reaction product was mixed with 1:1 EtOAc/hexane and was washed several times with water. The organic extract was dried over NaCl/MgSO₄, filtered through celite, and then condensed by rotary evaporation to yield a light yellow solid. This material was crystallized from 4:1 EtOAc/hexane to yield 276 mg of 2-(9H-fluoren-9-ylidene)ethanol as a white solid.

### Step 2: (9H-Fluoren-9-ylidene)acetaldehyde

A RB flask with a magnetic stir bar was charged with 2-(9H-fluoren-9-ylidene)ethanol (0.270 g, 1.29 mmol), DCM (15 mL) and Dess-Martin periodinane (848 mg, 2.0 mmol). The flask was sealed under a nitrogen atmosphere and the reaction stirred at RT. After 90 min, the reaction was quenched by the addition of dilute NaOH:water followed by Et₂O extraction. The combined Et₂O extracts were dried over NaCl/MgSO₄, filtered, and rotary evaporated in vacuo to yield (9H-Fluoren-9-ylidene)acetaldehyde as an orange colored solid (256 mg).

### Step 3: (R)—N-(2-(9H-fluoren-9-ylidene)ethyl)-1-(4-fluorophenyl)ethanamine (Compound 61)

A RB flask with a magnetic stir bar was charged with (9H-Fluoren-9-ylidene)acetaldehyde (0.256 g, 1.24 mmol), 15 mL DCE, (R)-1-(p-fluorophenyl)ethanamine (0.173 g, 1.24 mmol), and phosphotungstic acid hydrate (23 mg). The reaction was stirred at room temperature for 10 minutes before addition of NaBH(AcO)₃ (0.590 g, 2.78 mmol). The reaction was then stirred at RT overnight and then worked up by addition of 10% NaOH solution and Et₂O extraction. The combined Et₂O layers were washed with brine, dried over NaCl/MgSO₄ and rotary evaporated under reduced pressure to give the product as yellow colored oil. The crude oil was purified by flash column chromatography on 15 g of silica gel eluding with 20-40% EtOAc/hexane gradient to afford 118 mg of (R)—N-(2-(9H-fluoren-9-ylidene)ethyl)-1-(4-fluorophenyl)ethanamine as a viscous oil.

61:—¹H NMR (CDCl₃, 400 MHz): δ 6.9-7.74 (m, 13H), 6.73 (t, 1H), 3.76-4.0 (m, 2H), 3.85 (q, 1H), 1.45 (d, 3H)

Exposure of a stirred Et₂O/hexane solution of (R)—N-(2-(9H-fluoren-9-ylidene)ethyl)-1-(4-fluorophenyl)ethanamine to HCl gas gave an immediate precipitate of (R)—N-(2-(9Hfluoren-9-ylidene)ethyl)-1-(4-fluorophenyl)ethanamine hydrochloride salt as a yellow solid.

### Example 56

### (R)—N-(1-(4-methoxyphenyl)ethyl)-3,3-diphenylprop-2-en-1-amine (Compound 62) and (R)—N-(1-(4-methoxyphenyl)ethyl)-3,3-diphenylprop-2-en-1-amine (Compound 68)

A RB flask with magnetic stir bar was charged with DCE (5 mL), acetic acid (110 mg, 1.8 mmol), R-(+)-p-methoxy-a-methylbenzyl amine (0.238 g, 1.57 mmol) and β-phenylcinnamylaldehyde (0.328 g, 1.57 mmol). To the stirred reaction solution under a nitrogen atmosphere was added NaBH(OAc)₃ (0.5 g, 2.35 mmol). The flask was sealed under nitrogen and stirred at RT overnight. The reaction was worked up by the addition of excess 1 N NaOH and Et₂O extraction. The combined Et₂O extracts were washed with brine, dried (MgSO₄), filtered, and condensed by rotary evaporation to yield an oil. This material was further purified by silica gel chromatography (15 g) using 20-100% EtOAc/hexane as the eluant to yield (R)—N-(1-(4-methoxyphenyl)ethyl)-3,3-diphenylprop-2-en-1-amine (233 mg) and the side product (R)—N-(1-(4-methoxyphenyl)ethyl)-3,3-diphenylprop-2-en-1-amine (29 mg).

62:—¹H NMR (CDCl₃, 400 MHz): δ 7.06-7.34 (m, 12H), 6.81 (d, 2H), 6.16 (t, 1H), 3.54 (q, 1H), 3.76 (s, 3H), 3.16 (d, 2H), 1.28 (d, 3H)

68:—¹H NMR (CDCl₃, 400 MHz): δ 7.03-7.32 (m, 22H), 6.76 (d, 2H), 6.12 (t, 2H), 3.83 (q, 1H), 3.75 (s, 3H), 3.15 (d, 4H), 1.14 (d, 3H)

Exposure of a stirred Et₂O solution of (R)—N-(1-(4-methoxyphenyl)ethyl)-3,3-diphenylprop-2-en-1-amine to HCl gas gave an immediate precipitate of (R)—N-(1-(4-methoxyphenyl)ethyl)-3,3-diphenylprop-2-en-1-amine hydrochloride salt as a light cream colored solid.

### Example 57

### (R)-3,3-diphenyl-N-(1-(p-tolyl)ethyl)prop-2-en-1-amine (Compound 63) and (R)—N-(3,3-diphenylallyl)-3,3-diphenyl-N-(1-(p-tolyl)ethyl)prop-2-en-1-amine (Compound 69)

A RB flask with magnetic stir bar was charged with DCE (6 mL), phosphotungstic acid hydrate (24 mg), R-(+)-p-methyl-a-methylbenzyl amine (0.195 g, 1.44 mmol) and β-phenylcinnamylaldehyde (0.300 g, 1.44 mmol). To the stirred reaction solution under a nitrogen atmosphere was added NaBH(OAc)₃ (0.458 g, 2.16 mmol). The flask was sealed under nitrogen and stirred at RT overnight. The reaction was worked up by the addition of excess 1 N NaOH and Et₂O extraction. The combined Et₂O extracts were washed with brine, dried MgSO₄, filtered, and condensed by rotary evaporation to yield an oil. This material was further purified by silica gel chromatography (15 g) using 20-100% EtOAc/hexane as the eluant to yield (R)-3,3-diphenyl-N-(1-(p-tolyl)ethyl)prop-2-en-1-amine (158 mg) and the side product (R)—N-(3,3-diphenylallyl)-3,3-diphenyl-N-(1-(ptolyl)ethyl)prop-2-en-1-amine (372 mg).

63:—¹H NMR (CDCl₃, 400 MHz): δ 7.0-7.36 (m, 14H), 6.14 (t, 1H), 3.70 (q, 1H), 3.15 (d, 2H), 2.27 (s, 3H), 1.26 (d, 3H)

69:—¹H NMR (CDCl₃, 400 MHz): δ 7.05-7.14 (m, 24H), 6.14 (t, 2H), 3.85 (q, 1H), 3.19 (d, 4H), 2.31 (s, 3H), 1.14 (d, 3H)

Exposure of a stirred Et₂O solution of (R)-3,3-diphenyl-N-(1-(p-tolyl)ethyl)prop-2-en-1-amine to HCl gas gave an immediate precipitate of (R)-3,3-diphenyl-N-(1-(ptolyl)ethyl)prop-2-en-1-amine hydrochloride salt as a white colored solid.

### Example 58

### (R)—N-(1-(3-chlorophenyl)ethyl)-3,3-diphenylprop-2-en-1-amine (Compound 64) and (R)—N-(1-(3-chlorophenyl)ethyl)-N-(3,3-diphenylallyl)-3,3-diphenylprop-2-en-1-amine (Compound 70)

A RB flask with magnetic stir bar was charged with DCE (15 mL), phosphotungstic acid hydrate (23 mg), R-(+)-p-chloro-a-methylbenzyl amine (0.187 g, 1.2 mmol) and β-phenylcinnamylaldehyde (0.250 g, 1.2 mmol). To the stirred reaction solution under a nitrogen atmosphere was added NaBH(OAc)₃ (0.381 g, 1.8 mmol). The flask was sealed under nitrogen and stirred at RT overnight. The reaction was worked up by the addition of excess 0.5 N NaOH and Et₂O extraction. The combined Et₂O extracts were washed with brine, dried (MgSO₄), filtered, and condensed by rotary evaporation to yield an oil. This material was further purified by silica gel chromatography (15 g) using 20-100% EtOAc/hexane as the eluant to yield (R)—N-(1-(3-chlorophenyl)ethyl)-3,3-diphenylprop-2-en-1-amine (250 mg) and the side product (R)—N-(3,3-diphenylallyl)-3,3-diphenyl-N-(1-(3-chlorophenyl)ethyl)prop-2-en-1-amine (127 mg).

64:—¹H NMR (CDCl₃, 400 MHz): δ 6.99-7.34 (m, 14H), 6.12 (t, 1H), 3.69 (q, 1H), 3.13 (d, 2H), 1.27 (d, 3H)

70:—¹H NMR (CDCl₃, 400 MHz): δ 6.99-7.33 (m, 24H), 6.10 (t, 2H), 3.82 (q, 1H), 3.14 (d, 4H), 1.08 (d, 3H)

Exposure of a stirred Et₂O solution of (R)—N-(1-(3-chlorophenyl)ethyl)-3,3-diphenylprop-2-en-1-amine to HCl gas gave an immediate precipitate of (R)—N-(1-(3-chlorophenyl)ethyl)-3,3-diphenylprop-2-en-1-amine hydrochloride salt as a white colored solid.

### Example 59

### (R)—N-(1-(2-methoxyphenyl)ethyl)-3,3-diphenylprop-2-en-1-amine (Compound 65) and (R)—N-(3,3-diphenylallyl)-N-(1-(2-methoxyphenyl)ethyl)-3,3-diphenylprop-2-en-1-amine (Compound 72)

A RB flask with magnetic stir bar was charged with DCE (13 mL), phosphotungstic acid hydrate (27 mg), R-(+)-o-methoxy-a-methylbenzyl amine (0.181 g, 1.2 mmol) and β-phenylcinnamylaldehyde (0.250 g, 1.2 mmol). To the stirred reaction solution under a nitrogen atmosphere was added NaBH(OAc)₃ (0.510 g, 2.4 mmol). The flask was sealed under nitrogen and stirred at RT overnight. The reaction was worked up by the addition of excess 0.5 N NaOH and Et₂O extraction. The combined Et₂O extracts were washed with brine, dried NaCl/MgSO₄, filtered, and condensed by rotary evaporation to yield an oil. This material was further purified by silica gel chromatography (15 g) using 20-100% EtOAc/hexane as the eluant to yield (R)—N-(1-(2-methoxyphenyl)ethyl)-3,3-diphenylprop-2-en-1-amine (260 mg) and the side product (R)—N-(3,3-diphenylallyl)-N-(1-(2-methoxyphenyl)ethyl)-3,3-diphenylprop-2-en-1-amine (116 mg).

65:—¹H NMR (CDCl₃, 400 MHz): δ 6.77-7.30 (m, 14H), 6.18 (t, 1H), 4.10 (q, 1H), 3.57 (s, 3H), 3.16 (m, 2H), 1.29 (d, 3H)

72:—¹H NMR (CDCl₃, 400 MHz): δ 6.81-7.19 (m, 22H), 6.76 (d, 2H), 6.17 (t, 2H), 4.33 (q, 1H), 3.58 (s, 3H), 3.29 (m, 4H), 1.15 (d, 3H)

Exposure of a stirred Et₂O solution of (R)—N-(1-(2-methoxyphenyl)ethyl)-3,3-diphenylprop-2-en-1-amine to HCl gas gave an immediate precipitate of (R)—N-(1-(2-methoxyphenyl)ethyl)-3,3-diphenylprop-2-en-1-amine hydrochloride salt as a white colored solid.

### Example 60

### (S)-2-((3,3-diphenylallyl)amino)-2-phenylethanol (Compound 66) and (S)-2-(bis(3,3-diphenylallyl)amino)-2-phenylethanol (Compound 71)

A RB flask with magnetic stir bar was charged with DCE (13 mL), phosphotungstic acid hydrate (24 mg), (S)-(+)-2-phenylglycinol (0.165 g, 1.2 mmol) and β-phenyl-cinnamylaldehyde (0.250 g, 1.2 mmol). To the stirred reaction solution under a nitrogen atmosphere was added NaBH(OAc)₃ (0.510 g, 2.4 mmol). The flask was sealed under nitrogen and stirred at RT overnight. The reaction was worked up by the addition of excess 0.5 N NaOH and Et₂O extraction. The combined Et₂O extracts were washed with brine, dried NaCl/MgSO₄, filtered, and condensed by rotary evaporation to yield an oil. This material was further purified by silica gel chromatography (15 g) using 20-100% EtOAc/hexane as the eluant to yield (S)-2-((3,3-diphenylallyl)amino)-2-phenylethanol (259 mg) and the side product (S)-2-(bis(3,3-diphenylallyl)amino)-2-phenylethanol (108 mg).

66:—¹H NMR (CDCl₃, 400 MHz): δ 7.02-7.32 (m, 15H), 6.12 (t, 1H), 3.68 (m, 2H), 3.49 (m, 1H), 3.23(m, 2H)

71:—¹H NMR (CDCl₃, 400 MHz): δ 7.0-7.34 (m, 30H), 6.12 (t, 2H), 3.68 (m, 2H), 3.49 (m, 1H), 3.23(m, 4H)

### Example 61

### (R)—N-(3,3-diphenylallyl)-2,3-dihydro-1H-inden-1-amine (Compound 67) and (R)—N,N-bis(3,3-diphenylallyl)-2,3-dihydro-1H-inden-1-amine (Compound 73)

A RB flask with magnetic stir bar was charged with DCE (12 mL), phosphotungstic acid hydrate (26 mg), R-(+)-1-aminoindane (0.162 g, 1.2 mmol) and (3-phenylcinnamylaldehyde (0.250 g, 1.2 mmol). To the stirred reaction solution under a nitrogen atmosphere was added NaBH(OAc)₃ (0.510 g, 2.4 mmol). The flask was sealed under nitrogen and stirred at RT overnight. The reaction was worked up by the addition of excess 0.5 N NaOH and Et₂O extraction. The combined Et₂O extracts were washed with brine, dried NaCl/MgSO₄, filtered, and condensed by rotary evaporation to yield an oil. This material was further purified by silica gel chromatography (15 g) using 20-100% EtOAc/hexane as the eluant to yield (R)—N-(3,3-diphenylallyl)-2,3-dihydro-1H-inden-1-amine (148 mg) and the side product (R)—N,N-bis(3,3-diphenylallyl)-2,3-dihydro-1H-inden-1-amine (205 mg).

67:—¹H NMR (CDCl₃, 400 MHz): δ 7.08-7.42 (m, 14H), 6.23 (t, 1H), 4.23 (m, 2H), 3.42 (m, 2H), 2.93(m, 1H), 2.75(m, 1H), 2.26(m, 1H), 1.72(m, 1H)

73:—¹H NMR (CDCl₃, 400 MHz): δ 7.086-7.45 (m, 24H), 6.21 (t, 2H), 4.21 (m, 2H), 3.42 (m, 4H), 2.93(m, 1H), 2.75(m, 1H), 2.25(m, 1H), 1.74(m, 1H)

Exposure of a stirred Et₂O solution of (R)—N-(3,3-diphenylallyl)-2,3-dihydro-1H-inden-1-amine to HCl gas gave an immediate precipitate of (R)—N-(3,3-diphenylallyl)-2,3-dihydro-1H-inden-1-amine hydrochloride salt as a yellow solid.

### Example 62

### Results of Proliferation Assay for Five Different Endometrial Cancer Cell Lines

Based upon the concentration-response curves of five different compounds (42, 68, 70, 72, and 73) were tested for their effects on cell proliferation of five different endometrial cell lines. These experiments were carried out as described in the Methods and Materials section. Based upon the concentration-response curves, the EC₅₀ values for the inhibitory effects of the compound on the particular cancer cell lines are described in the table below (Table 4).

TABLE 4
EC₅₀ of Fendiline Derivatives for Five
Different Endometrial Cancer Cell Lines

| | KLE | ESS-1 | Hec-1A | | Hec-1B | | Hec50 | |
|---|---|---|---|---|---|---|---|---|
| Compound 42 | ND | ND | 7.389 | nM | 523.9 | nM | 422.6 | nM |
| Compound 68 | ND | ND | 210.8 | nM | ND | | 1.58 | nM |
| Compound 70 | ND | ND | 38.16 | nM | 10727 | nM | 3306 | nM |
| Compound 72 | ND | ND | 107.4 | nM | 658.2 | nM | 554.6 | nM |
| Compound 73 | ND | ND | 19.65 | nM | 330.3 | nM | 411.7 | nM |

### Example 63

### Results of Proliferation Assay for Five Different Pancreatic Cancer Cell Lines

Based upon the concentration-response curves of five different compounds (42, 68, 70, 72, and 73) were tested for their effects on cell proliferation of five different pancreatic cell lines. These experiments were carried out as described in the Methods and Materials section. Based upon the concentration-response curves, the EC₅₀ values for the inhibitory effects of the compound on the particular cancer cell lines are described in the table below (Table 5).

TABLE 5
EC₅₀ of Fendiline Derivatives for Five
Different Pancreatic Cancer Cell Lines

| | BxPC- | MiaPaCa- | | MPanc- | | | | | |
|---|---|---|---|---|---|---|---|---|---|
| | 3 | 2 | | 96 | | MOH | | HPAC | |
| Compound 42 | ND | 2414 | nM | 1115 | nM | 1035 | nM | 944.3 | nM |
| Compound 68 | ND | 1150 | nM | 911.5 | nM | 930.6 | nM | 104.9 | nM |
| Compound 70 | ND | 1496 | nM | 814 | nM | 663.3 | nM | 220.4 | nM |
| Compound 72 | ND | 692.1 | nM | 2572 | nM | 528.4 | nM | 1718 | nM |
| Compound 73 | ND | 894.4 | nM | 239.1 | nM | 615.7 | nM | 346.5 | nM |

### REFERENCES

The following references, to the extent that they provide exemplary procedural or other details supplementary to those set forth herein, are specifically incorporated herein by reference.
U.S. Provisional Patent Application No. 61/640,451
Hancock, “Ras proteins: different signals from different locations,” Nat Rev Mol Cell Biol, 4, 373-84, 2003.
Hancock and Parton, “Ras plasma membrane signaling platforms,” Biochem J, 389, 1-11, 2005.
Handbook of Pharmaceutical Salts: Properties, and Use, P. H. Stahl & C. G. Wermuth eds., Verlag Helvetica Chimica Acta, 2002.
Kukovetz, et al., “Single dose pharmacokinetics of fendiline in humans,” Eur J Drug Metab Pharmacokinet., 7(2):105-10, 1982.
Lückhoff, et al., “Effects of the calmodulin antagonists fendiline and calmidazolium on aggregation, secretion of ATP, and internal calcium in washed human platelets,” Naunyn Schmiedebergs Arch Pharmacol., 343(1):96-101, 1991.
March's Advanced Organic Chemistry: Reactions, Mechanisms, and Structure (2007)
Pharmaceutical dosage form tablets, Lieberman, et al. eds., CRC Press, 1989.
Pharmaceutical dosage forms and drug delivery systems, Ansel, et al., eds., Lippincott Williams and Wilkins, 1995.
Remington—The science and practice of pharmacy, Alfonso R. Gennaro, ed, 20^{th} Edition, Lippincott Williams and Wilkins, 2000.
Weyhenmeyer et al., “Tolerance and pharmacokinetics of oral fendiline,” Arzneimittelforschung., 37(1):58-62, 1987.

1. A compound of the formula: 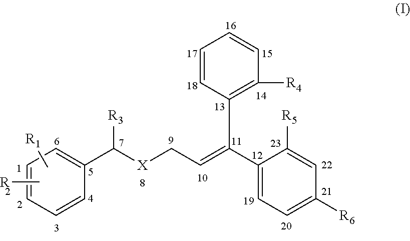
wherein:
X is —O— or —NY₁—;
wherein Y₁ is hydrogen, alkyl_{(C≦6)}, aralkyl_{(C≦18)}, aralkenyl_{(C≦18)}, or a substituted version of any of these groups;
R₁ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups;
R₂ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups;
R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, acyl_{(C≦12)}, alkoxy_{(C≦12)}, aryloxy_{(C≦12)}, aralkoxy_{(C12)}, heteroaryloxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, amido_{(C≦12)}, or a substituted version of any of these groups;
R₁ and R₃, taken together, are alkanediyl_{(C≦6)} or substituted alkanediyl_{(C≦6)} between carbon atoms 6 and 7;
R₄ and R₅ are each hydrogen; or
R₄ and R₅, taken together, are a covalent single bond, —O—, —S—, alkanediyl_{(C≦12)}, or alkenediyl_{(C≦12)} between carbon atoms 14 and 23; and
R₆ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups;
with the proviso that if R₁ and R₂ are both hydrogen, then R₃ is substituted alkyl_{(C≦6)};
or a pharmaceutically acceptable salt or tautomer thereof.

2. The compound of claim 1, further defined by the formula: 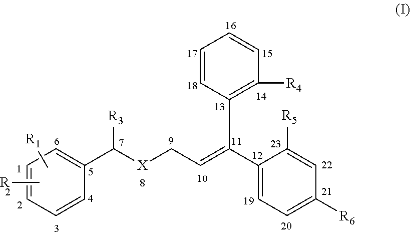
wherein:
X is —O— or —NH—;
R₁ is hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups;
R₂ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups;
R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, acyl_{(C≦12)}, alkoxy_{(C≦12)}, aryloxy_{(C≦12)}, aralkoxy_{(C≦12)}, heteroaryloxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, amido_{(C≦12)}, or a substituted version of any of these groups;
R₄ and R₅ are each hydrogen or are taken together and are a covalent single bond, —O—, —S—, alkanediyl_{(C≦12)}, or alkenediyl_{(C≦12)}; and
R₆ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups;
or a pharmaceutically acceptable salt or tautomer thereof.

3. The compound of claim 1, wherein X is —NY₁—.

4.-9. (canceled)

10. The compound of claim 1, wherein R₁ is alkoxy_{(C≦6)}.

11. The compound of claim 10, wherein R₁ is methoxy.

12.-15. (canceled)

16. The compound of claim 1, wherein R₂ is hydrogen.

17. The compound of claim 1, wherein R₃ is alkyl_{(C≦12)} or substituted alkyl_{(C≦12)}.

18. The compound of claim 17, wherein R₃ is alkyl_{(C≦12)}.

19. The compound of claim 18, wherein R₃ is methyl.

20.-21. (canceled)

22. The compound of claim 1, wherein R₄ and R₅ are each hydrogen.

23.-25. (canceled)

26. The compound of claim 1, wherein R₆ is hydrogen.

27. The compound of claim 1, further defined as: 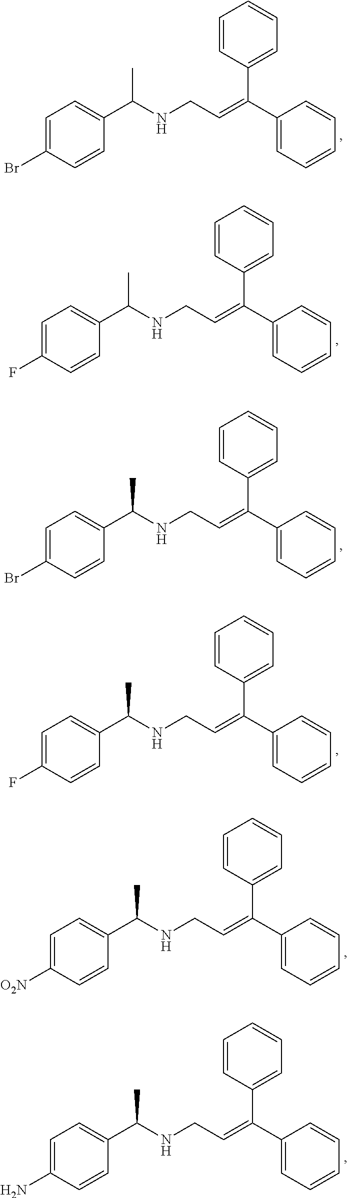 [SMILES: CC(NCC=C(c1ccccc1)c1ccccc1)c1ccc(Br)cc1 ; CC(NCC=C(c1ccccc1)c1ccccc1)c1ccc(F)cc1 ; C[C@@H](NCC=C(c1ccccc1)c1ccccc1)c1ccc(Br)cc1 ; C[C@@H](NCC=C(c1ccccc1)c1ccccc1)c1ccc(F)cc1 ; C[C@@H](NCC=C(c1ccccc1)c1ccccc1)c1ccc([N+](=O)[O-])cc1 ; C[C@@H](NCC=C(c1ccccc1)c1ccccc1)c1ccc(N)cc1] 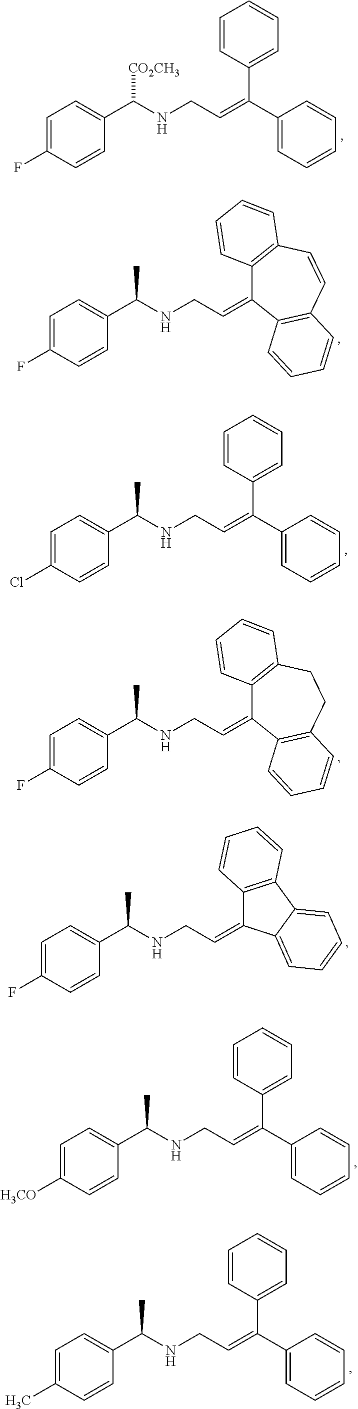 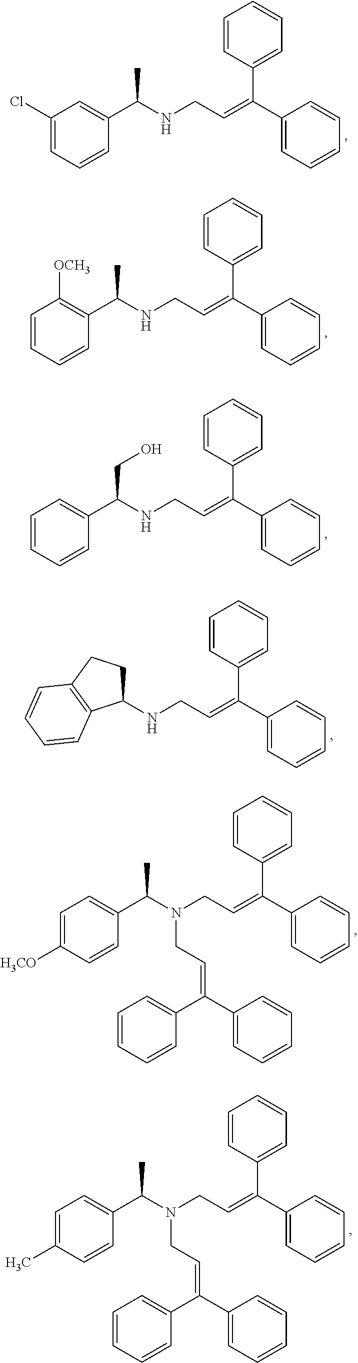 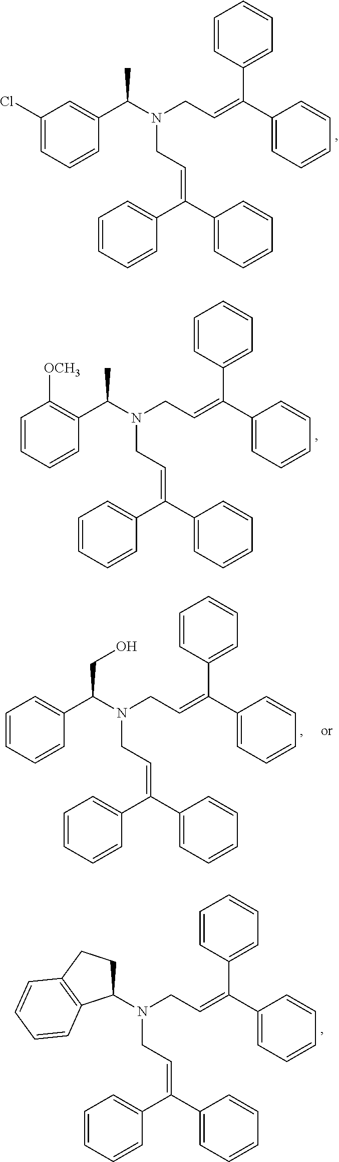
or a pharmaceutically acceptable salt or tautomer thereof.

28. (canceled)

29. A compound of the formula: 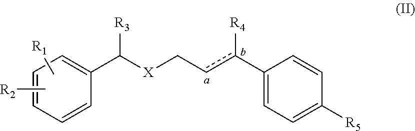
wherein:
X is —O— or —NH—;
R₁ and R₂ are each independently hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups; and
R₃ is acyl_{(C≦12)}, substituted acyl_{(C≦12)}, heteroaryl_{(C≦12)}, or substituted heteroaryl_{(C≦12)};
R₄ is hydrogen or aryl_{(C≦12)}; and
R₅ is hydrogen, hydroxy, amino, halo, nitro or cyano; or alkyl_{(C≦6)}, acyl_{(C≦6)}, alkoxy_{(C≦6)}, acyloxy_{(C≦6)}, alkylamino_{(C≦6)}, dialkylamino_{(C≦6)}, amido_{(C≦6)}, or a substituted version of any of these groups;
or a pharmaceutically acceptable salt or tautomer thereof.

30.-85. (canceled)

86. The compound of claim 1, wherein the compound is in the form of a pharmaceutically acceptable salt.

87. The compound of claim 86, wherein the compound is in the form of a hydrochloride salt.

88. The compound of claim 1, wherein the compound is not a salt.

89. A pharmaceutical composition comprising the compound of claim 1 and a pharmaceutically acceptable carrier.

90. The pharmaceutical composition of claim 89, wherein the composition is formulated for oral administration.

91. The pharmaceutical composition of claim 89, further comprising one or more pharmaceutically acceptable excipients.

92. The pharmaceutical composition of claim 89, wherein the composition is formulated for controlled release.

93. A method of treating a proliferative disorder, wherein the method comprises administering to a patient in need thereof a therapeutically effective amount of a compound of claim 1.

94-105. (canceled)
